(19) United States
(12) Patent Application Publication
(10) Pub. No.: US 2025/0388958 A1
(43) Pub. Date: Dec. 25, 2025

(54) COMPOSITIONS, KITS, AND METHODS FOR DETECTING NUCLEIC ACIDS USING INTRA-CHANNEL MULTIPLEXING

(71) Applicant: LIFE TECHNOLOGIES CORPORATION, Carlsbad, CA (US)

(72) Inventors: Mark E. Shannon, San Francisco, CA (US); Carmen Gjerstad, Millbrae, CA (US); Harrison Leong, San Francisco, CA (US); Mohammad Mehdi Zahedi, Dublin, CA (US); Brian J. Evans, Mountain View, CA (US); Khairuzzaman Bashar Mullah, Union City, CA (US)

(73) Assignee: LIFE TECHNOLOGIES CORPORATION, Carlsbad, CA (US)

(21) Appl. No.: 18/879,446

(22) PCT Filed: Jun. 29, 2023
(86) PCT No.: PCT/US2023/069407

§ 371 Date: Dec. 27, 2024

Related U.S. Application Data
(60) Provisional application No. 63/356,874, filed on Jun. 29, 2022.
(60) Provisional application No. 63/356,863, filed on Jun. 29, 2022.
(60) Provisional application No. 63/408,665, filed on Sep. 21, 2022.
(60) Provisional application No. 63/453,546, filed on Mar. 21, 2023.

(51) Int. Cl. C12Q 1/686; C12Q 1/6809; C12Q 1/6816; C12Q 1/6851; C12Q 1/6876

(52) U.S. Cl. CPC C12Q 1/686; C12Q 1/6809; C12Q 1/6816; C12Q 1/6851; C12Q 1/6876; C12Q 2600/16

(57) ABSTRACT

Disclosed are compositions, kits, and methods that enable intra-channel multiplexing by enabling determination of separate detectable signals, each associated with a different assay target, within the same detection channel. The multiple detectable signals can be separately resolved and independently analyzed to enable detection and/or quantification of each respective target. Enabling multiple targets to be assayed within the same detection channel increases the plexy of multiplex assays without relying on additional dyes and concomitant issues of increased spectral overlap.

### CROSS-REFERENCE TO RELATED APPLICATIONS

This application claims priority to U.S. Provisional Patent Application No. 63/453,546, filed Mar. 21, 2023; and to U.S. Provisional Patent Application No. 63/408,665, filed Sep. 21, 2022; and to U.S. Provisional Patent Application No. 63/356,863, filed Jun. 29, 2022; and to U.S. Provisional Patent Application No. 63/356,874, filed Jun. 29, 2022, the entirety of each of which is incorporated herein by this reference.

### TECHNICAL FIELD

This disclosure is directed to compositions, kits, and methods that enable multiplexing by enabling determination of signals having similar or the same spectral properties but that are each associated with a different assay target. Aspects of the disclosure further relate to compositions, kits, and methods that enable multiplexing by enabling determination of signals associated with different assay targets using the same detection channel (e.g., within the same fluorescence channel).

### INTRODUCTION

Nucleic acid detection assays are often carried out by adding a sample that is suspected of including one or more target nucleic acids to a reaction mixture. The reaction mixture can include one or more detectable labels each designed to associate with a different target nucleic acid and generate a signal that corresponds to the amount of target nucleic acid in the reaction mixture. In a “singleplex” assay, the reaction mixture includes a single detectable label designed to associate with a single target. Conversely, in a “multiplex” assay, the reaction mixture includes multiple, different detectable labels each typically designed to be specific to a different target nucleic acid. Multiplex assays are therefore capable of detecting multiple different targets in a single reaction mixture. In some applications, the detectable labels are fluorescent dyes integrated with a nucleic acid probe, a primer, or some other nucleic acid molecule designed to specifically hybridize with the corresponding target nucleic acid with which it is designed to associate.

In various multiplex nucleic acid detection assays, each detectable label is assigned to a different target nucleic acid. The presence and/or amount of each target nucleic acid can then be determined by measuring the signal emitted from the detectable label in separate “detection channels” each corresponding to a specific property of the corresponding emitted signal. For example, in the context of fluorescence-emitting dyes as a detectable label, the separate detection channels can correspond to the emission wavelength spectrum associated with each dye. However, there can be a substantial amount of overlap in the emission spectra of the different dyes. Increased overlap in emission spectra increases the difficulty in resolving the separate detected emission (e.g., fluorescence) signals and thus increases the difficulty in detecting and/or quantifying the respective targets. Excessive overlap can require, for example, complex deconvolution algorithms to sufficiently resolve the separate fluorescence signals.

While multiplexed dyes can be selected with the intent to minimize spectral overlap, the finite nature of the emission spectrum places practical limits on the number of separate dyes that can be combined in the same multiplex assay, at least without resorting to increasingly complex reaction protocols and backend deconvolution requirements. As a result, at present, there are significant limitations to the number of different targets that can be detected and/or measured in a multiplex assay. Accordingly, there is an ongoing need for compositions, kits and methods capable of increasing the “plexy” of detection assays. Moreover, it may be desirable to otherwise use dyes that have some degree of overlap in emission spectra and/or that use the same dye for different target nucleic acids.

Challenges can arise when implementing multiplexing for determining the relative amounts of different target nucleic acids in a sample. In particular, using detectable labels that have overlapping emission spectra can be challenging to determine the respective contributions of each label individually and thus of the respective different target nucleic acids with which they are associated.

A need exists to provide more robust techniques for carrying out multiplex nucleic acid detection assays, such as nucleic acid detection utilizing various polymerase chain reaction (PCR) assays for example.

### BRIEF DESCRIPTION OF THE DRAWINGS

Various objects, features, characteristics, and advantages of inventions within the scope of the present disclosure will become apparent and more readily appreciated from the following description of various embodiments, taken in conjunction with the accompanying drawings and the appended claims, all of which form a part of this specification. In the Drawings, like reference numerals may be utilized to designate corresponding or similar parts in the various figures, and the various elements depicted are not necessarily drawn to scale, wherein:

FIG. 1A illustrates emission spectra for various fluorescent dyes that can be used in nucleic acid detection assays and their associated detection channels;

FIG. 1B illustrates emission spectra for various fluorescent dyes that can be used in nucleic acid detection assays and their associated detection channels, with two of the dyes “Dye 1” and “Dye 2” having emission that can be detected within the same channel;

FIG. 2A is a schematic overview of a method for detecting multiple target nucleic acids within the same detection channel in accordance with various embodiments of the present disclosure;

FIG. 213 is a graph showing signal response over time for the method outlined in FIG. 2A when the reaction mixture is cycled between a first set of reaction conditions and a second set of reaction conditions;

FIG. 3A illustrates activity of a cleavable probe and a non-cleavable probe during annealing, extension, and denaturation steps of a thermal cycle, according to various embodiments;

FIG. 3B is a graph showing fluorescent signal response over time during thermal cycling of an amplification process that utilizes the cleavable and non-cleavable probes of FIG. 3A according to various embodiments;

FIG. 4A illustrates a process of using a primer with the tail, specific to a nucleic acid target, to form a template to which an extendable fluorogenic (“EF”) probe can hybridize;

FIG. 4B illustrates an example tailed forward primer, reverse primer, and EF probe that may be included in a reaction mixture, or a composition, for implementing the process of FIG. 4A;

FIG. 4C illustrates a three-stage thermal cycling method that may be utilized during an amplification process involving an EF probe and optionally a cleavable probe;

FIGS. 5A and 5B provide an overview of a method for detecting multiple target nucleic acids within the same detection channel in polymerase chain reaction (PCR) applications;

FIG. 6A illustrates fluorescent signals of a TaqMan probe and an extendable fluorogenic (EF) probe at extension and denaturation steps;

FIGS. 6B, 6C, and 6D illustrate results of a duplex assay test in which TaqMan and EF probes were designed to generate fluorescence signals in the same dye channel (FIG. 6B) or in different dye channels (FIGS. 6C and 6D);

FIG. 6E compares the EF-associated fluorescence signal after baseline adjustment (dRn) as derived using the results of the assay of FIG. 6B with the EF-associated fluorescence signal as directly measured in the assay of FIG. 6C;

FIG. 6F compares the TaqMan-associated fluorescence signal after baseline adjustment (dRn);

FIG. 7 illustrates the results of a 9-plex assay test that included 5 different detection channels/dyes, four of the channels with a corresponding TaqMan probe and an EF probe (each channel having a differing dye common to the TaqMan and EF probes in that channel) and one channel with only an TaqMan probe, according to embodiments of the present disclosure; and

FIG. 8 is a plot comparing the endpoint signal of partitions at 65° C. and at 95° C. following a dPCR process, showing that the partition signals fall into identifiable clusters that allow for estimation of concentration of different targets.

### DETAILED DESCRIPTION

### Selected List of Defined Terms

In the context of a nucleic acid probe and a target nucleic acid, the term “specifically interact” (and similar terms) indicates that the probe is designed to interact with the target to a greater degree than with non-target nucleic acids also present in the reaction mixture. For example, specific interaction may include hybridization of the probe, in whole or in part, with the corresponding target. The hybridization between the probe and target need not be 100%. For example, functionally effective interaction may be accomplished with probes having homology to their respective target of at least about 70%, at least about 75%, at least about 80%, at least about 85%, at least about 90%, at least about 95%, at least about 97%, at least about 98%, at least about 99%, or up to 100%.

As used herein, a “detection channel” is a specified, subset of the total range of possible values of detectable signals. For example, where the detectable signals are fluorescence signals, a detection channel (i.e., fluorescence channel or dye channel) can represent a wavelength band of specified size. A detection channel may, for example, have a band size of about 10-60 nm, depending on instrument features such as sensitivity and/or desired signal granularity. A detection channel can further include a discontinuous wavelengths or wavelength ranges. A detection channel may additionally or alternatively be defined according to the optical filter arrangement used to measure the detectable signals. Each different detection channel typically comprises a specific optical filter arrangement to block non-channel emissions. Thus, as a functional definition, each detectable signal within a given optical filter arrangement may be considered as being within the same detection channel. In some instances, different fluorescent labels (e.g., different chemical structures) are nonetheless detected with the same detection channel. As an example, the fluorescent dyes Cy5 and Alexa647 provide similar emission wavelengths and may be detected within the same channel.

As used herein, “substantially identical” signals are signals that are not clearly distinguishable from each other under the detection conditions being used. Optionally, the emission spectra of two substantially identical signals overlap to such an extent that each signal cannot be separately detected, such as where the composite emission spectrum does not show the presence of two distinct peaks. Optionally, “substantially identical fluorescence” emissions can be within similar wavelength bands. For example, a first fluorescence signal and a second fluorescence signal with substantially identical fluorescence may have emission peaks that differ by no more than about 10 nm, or no more than about 8 nm, or no more than about 6 nm, or no more than about 4 nm, or no more than about 2 nm, or no more than about 1 nm, or are substantially indistinguishable from one another by the detection instrument used to measure the fluorescence emissions. Additionally, or alternatively, fluorescence signals may be considered to have “substantially identical fluorescence” in applications where they are measured using the same detection apparatus, such as the same optical filter arrangement. In an embodiment, the substantially identical signals have substantially identical excitation/absorbance spectra, such that they cannot be subjected to excitation separately. Optionally, both labels are subjected to excitation during detection. Both labels can be simultaneously excited and/or detected.

As used herein with respect to signals, “substantial” indicates significantly above a background. For example, a “substantial signal” and/or a detectable signal that has “substantial fluorescence” is a signal significantly above a background (i.e., baseline) level, including a fluorescence signal that is significantly above a background/baseline level of fluorescence. This may be defined by a threshold value that separates background fluorescence from substantial fluorescence. The threshold value may vary according to particular testing protocols and application needs. In some embodiments (e.g., without a passive reference), the threshold is set at a ΔRn of about 1,000 to about 30,000, or about 2,000 to about 20,000, or about 3,000 to about 15,000 or about 4,000 to about 6,000, for example, or within a range having endpoints defined by any two of the foregoing values. In some embodiments (e.g., with a passive reference), the threshold is set at a ΔRn of about 0.01 to 0.5, for example. In some embodiments, the threshold value is some percentage above the baseline level, such as about 5 percent to about 10 percent above the baseline level.

A “background” or “baseline” level of signal (i.e., background/baseline level of fluorescence) during an amplification process may be determined according to methods known to those of skill in the art. As a non-limiting example, the baseline level may be determined as the median signal of the amplification cycles before exponential amplification occurs. For example, exponential amplification may be determined when the change in signal from one amplification cycle to the next exceeds a certain percentage indicative of exponential change.

As a corollary, a signal and/or fluorescence level that is not “substantial” according to the foregoing may be described herein as “negligible.” Similarly, with respect to probe binding, a probe is “substantially bound” to its target when it is bound significantly above background (e.g., above binding to a non-target). Optionally, at least 1%, 5%, 10%, 20%, 50% or 80% of the probe or the target is bound.

As used herein, a “cleavable” probe is a probe that is intended to be cleaved as a result of specific interaction of the probe with its respective target, and to cause a release of the corresponding label and an increase in the corresponding detectable signal as a result.

As used herein, a “non-cleavable” probe is a probe with a label that is intended to remain associated with the probe throughout the assay. In a non-cleavable probe, the corresponding detectable signal varies according to configuration changes of the probe rather than by release of the label from the probe. An extendable fluorogenic probe, such as a universal or hairpin extendable fluorogenic probe, as described in various embodiments, is an example of a non-cleavable probe.

The terms “detectable signal” and “label signal” are used synonymously herein. For example, a “first label signal” is the signal emitted by a first label of a first probe type and a “second label signal” is the signal emitted by a second label of a second probe type. A “total signal” is the total measured signal within a particular detection channel at a given time point or measurement point. Multiple different “detectable signals”/“label signals” may contribute to the same “total signal.” For example, a total signal may include signal generated by a first label of a first probe type and signal generated by a second label of a second probe type. In some embodiments, the signals are fluorescence signals, and terms such as “first fluorescence signal,” “second fluorescence signal,” and “total fluorescence signal” may be used as specific examples of the corresponding broader terms.

The term “spectral similarity” refers to the emission signal of detectable labels that have the same spectral profile or a substantially overlapping spectral profile. Thus, different probe types carrying the same detectable label or different probe types carrying different detectable labels with substantial spectral overlap in emission signal can both be considered probes with spectral similarity. In some implementations, detectable labels having spectral similarity can be detectable in a same optical detection channel, but other techniques can be used as well to detect the emission signals of such detectable labels. References made to substantially overlapping spectra should be understood to mean spectral similarity.

The term “end-point” as referring to a cycle is a designated cycle at which the PCR process is assumed to be completed and/or a designated cycle at which a signal threshold that is above background signal by a defined amount occurs. In various embodiments, an endpoint cycle in accordance with the present disclosure may range from 20 to 45 cycles, for example, from 30-40 cycles. However, the number of cycles to an endpoint cycle may change. For example, the number of cycles at an end-point cycle may be correlated to where the emission (e.g., fluorescence) signal indicative of amplification product reaches an approximate plateau. And “end-point signal” refers to an emission signal measured during an end-point cycle. The end-point signal can be measured at any designated, or chose, cycle.

The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene.” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol [symbol] denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

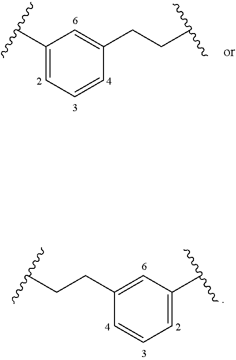 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂L, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₆ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₂ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both F and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D} etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In certain embodiments the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages; and peptide nucleic acid backbones and linkages. Other analog nucleic acids include those with positive backbones; non-ionic backbones, and non-ribose backbones. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. A residue of a nucleic acid, as referred to herein, is a monomer of the nucleic acid (e.g., a nucleotide). The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. A “nucleic acid moiety” as used herein is a monovalent form of a nucleic acid. In embodiments, the nucleic acid moiety is attached to the 3′ or 5′ position of a nucleotide or nucleoside.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

“Nucleotide,” as used herein, refers to a nucleoside-5′-phosphate (e.g., polyphosphate) compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may comprise bases such as adenine (A), cytosine (C), guanine (G), thymine (T), uracil (U), or analogues thereof, and may comprise 1, 2, 3, 4, 5, 6, 7, 8, or more phosphates in the phosphate group. Nucleotides may be modified at one or more of the base, sugar, or phosphate group. A nucleotide may have a label or tag attached (a “labeled nucleotide” or “tagged nucleotide”). In embodiments, the nucleotide is a deoxyribonucleotide. In embodiments, the nucleotide is a ribonucleotide. In embodiments, nucleotides comprise 3 phosphate groups (e.g., a triphosphate group).

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art). Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A “nucleoside” is structurally similar to a nucleotide, but is missing the phosphate moieties that are present in a nucleotide. An example of a nucleoside analogue would be one in which the label is linked to the base and there is no phosphate group attached to the sugar molecule. “Nucleoside,” as used herein, refers to a glycosyl compound consisting of a nucleobase and a 5-membered ring sugar (e.g., either ribose or deoxyribose). Nucleosides may comprise bases such as adenine (A), cytosine (C), guanine (G), thymine (T), uracil (U), or analogues thereof. Nucleosides may be modified at the base and/or and the sugar. In embodiments, the nucleoside is a deoxyribonucleoside. In embodiments, the nucleoside is a ribonucleoside.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity.

The term “polymerase,” as used herein, refers to any natural or non-naturally occurring enzyme or other catalyst that is capable of catalyzing a polymerization reaction, such as the polymerization of nucleotide monomers to form a nucleic acid polymer. Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). As used herein, the term “thermophilic nucleic acid polymerase” refers to a family of DNA polymerases (e.g., 9° NTM) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′->5′ direction, releasing deoxyribonucleoside 5′-monophosphates one after another.

The terms “determine,” “calculate,” and “estimate” are used synonymously herein. These terms are not intended to imply an exact level of measurement precision. Thus, where a value is “determined,” “calculated,” or “estimated” using the embodiments described herein, it will be understood that such a value may include some degree of inherent error due to factors such as detection instrument tolerances, rounding, chemical reaction variability, and other inherent measurement imperfections known and understood by those of skill in the art.

For any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

Overview of Multiplexing Utilizing Probes with Labels Having Spectral Similarity

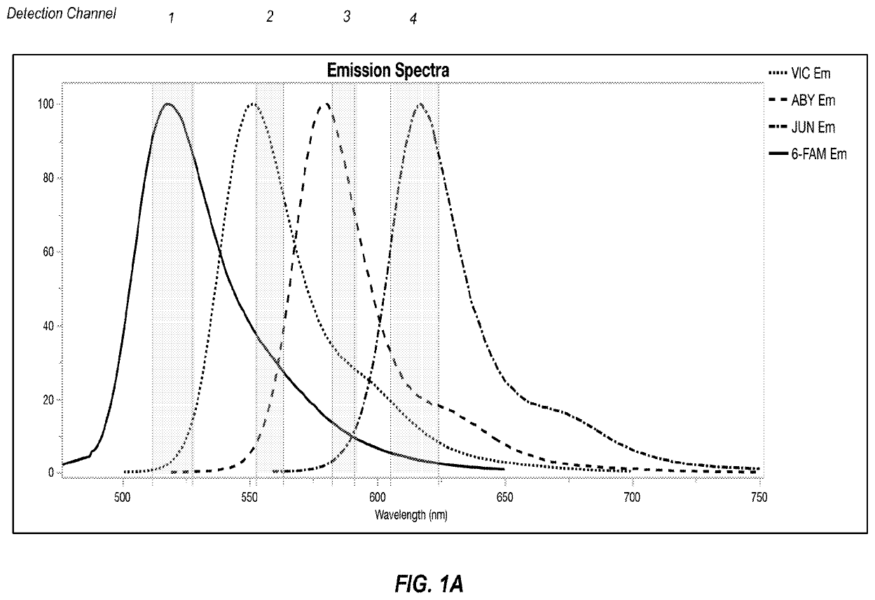

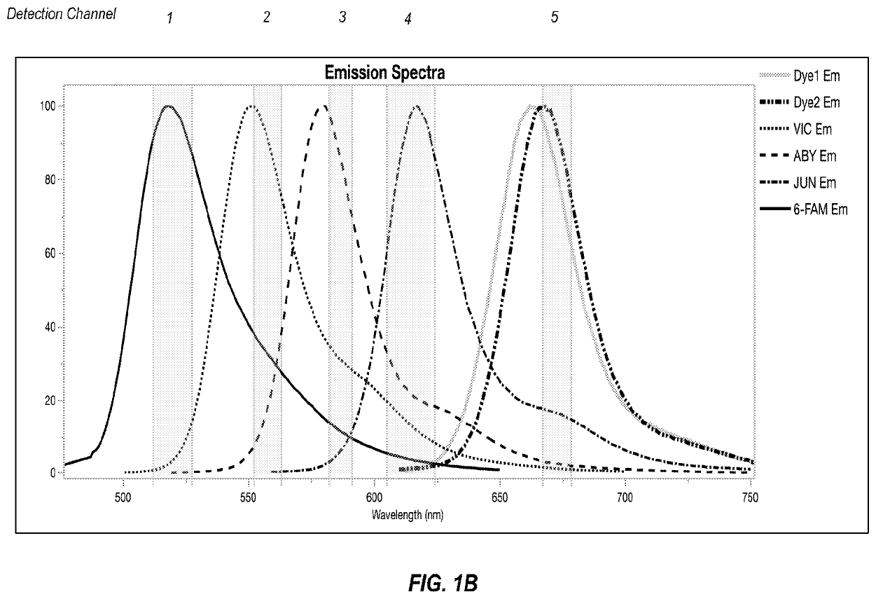

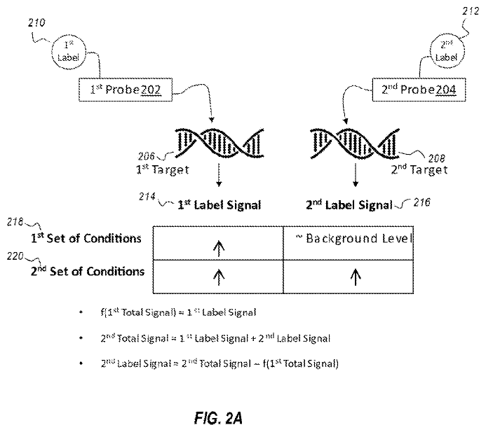

FIG. 1A illustrates emission spectra for various fluorescent dyes that can be used in nucleic acid detection assays. As discussed above, some multiplex assays assign each dye to a separate target, and then determine the presence and/or amount of each target by measuring the fluorescence signal, for example, in separate detection channels each corresponding to the emission wavelength of the corresponding dye. As shown in FIG. 1B, in some cases there can be a substantial amount of overlap in the emission spectra of the dyes. For example, Dye 1 and Dye 2 in FIG. 1B illustrate substantially overlapping emission spectra and that would be detectable by the same channel (channel 5) in FIG. 1B). While multiplexed dyes are typically selected with the intent to minimize spectral overlap, the finite nature of the emission spectrum places practical limits on the number of separate dyes that can be combined in the same multiplex assay when employing existing techniques, and therefore limits the number of different targets that can be detected and/or measured.

Embodiments described herein solve one or more of the foregoing problems by providing multiple detectable signals, each associated with a different assay target or set of targets, that have similar emission spectra, such as, for example that may correspond to detection in a same detection channel. The multiple detectable signals can be separately resolved and independently analyzed to thereby allow detection and/or quantification of each target. By allowing multiple targets to be assayed utilizing the same dye or dyes with spectral similarity, disclosed embodiments can beneficially increase the “plexy” (i.e., number of targets that can be detected and quantified) of multiplex assays without relying on additional dyes and concomitant issues of increased spectral overlap. For example, a common detection channel can be used to detect dyes having spectral similarity but that are intended for different targe analytes in accordance with aspects of the present disclosure. Similarly, embodiments described herein can beneficially decrease the number of separate dyes required in a multiplex assay without lowering the plexy of the assay. In addition, various embodiments can allow for the same dye to be used as a label for different target nucleic acids in a multiplex assay, including to use the same dye for different targets at the same time during the reaction. Further, various embodiments can allow for detection of the same dye in a same detection channel.

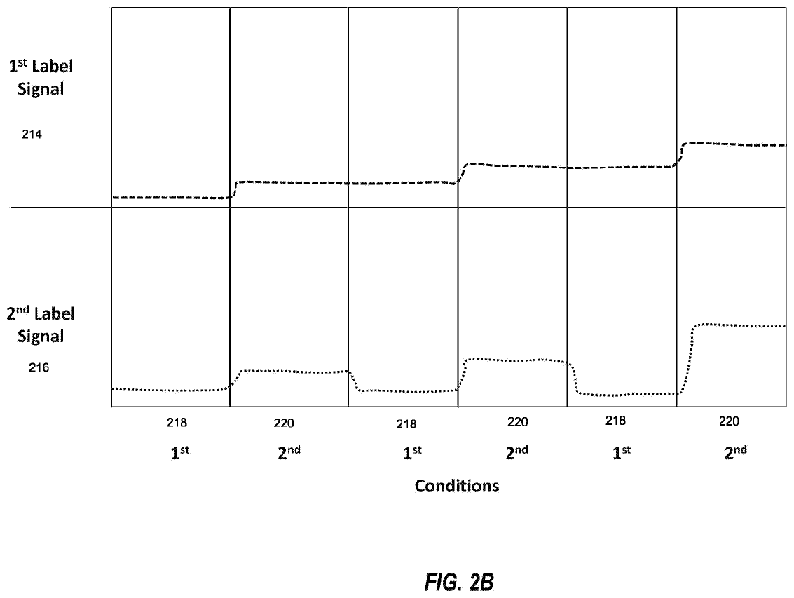

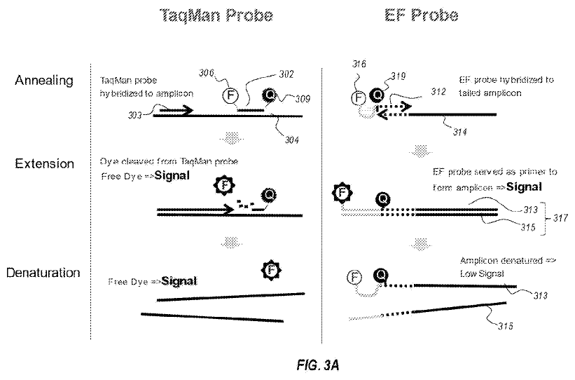

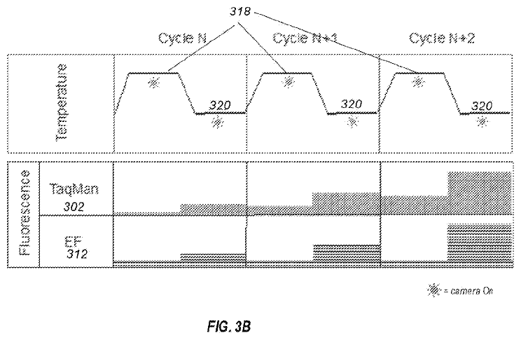

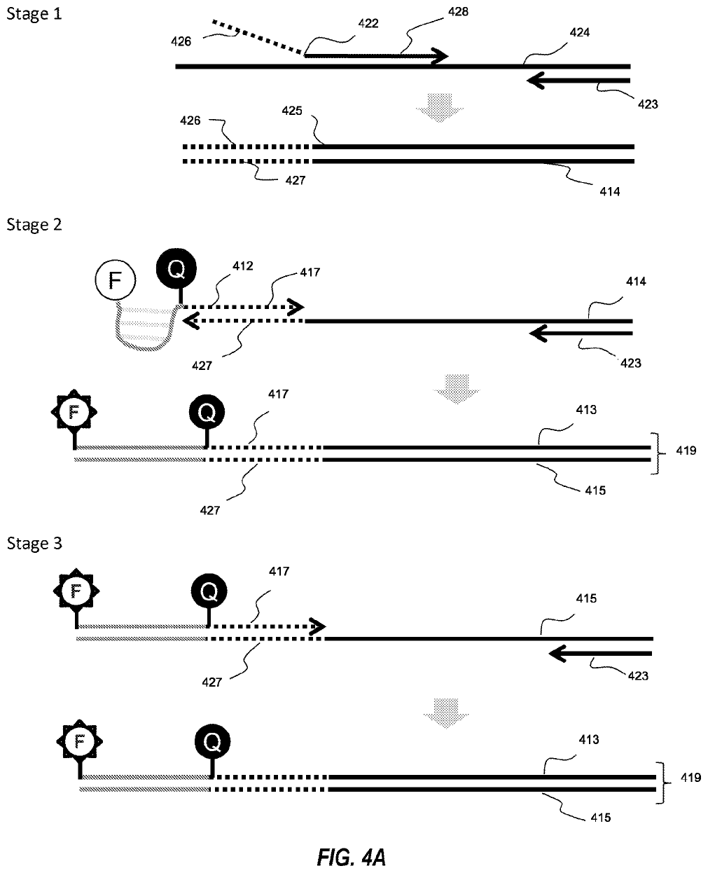

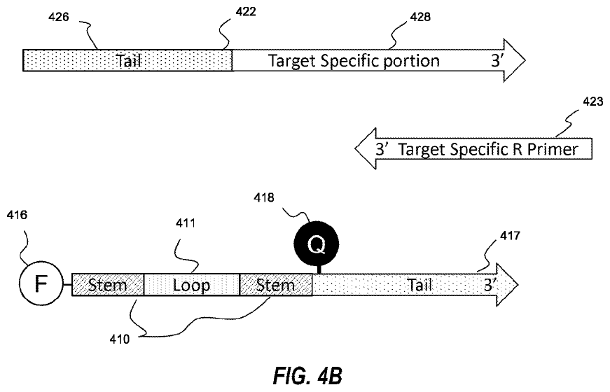

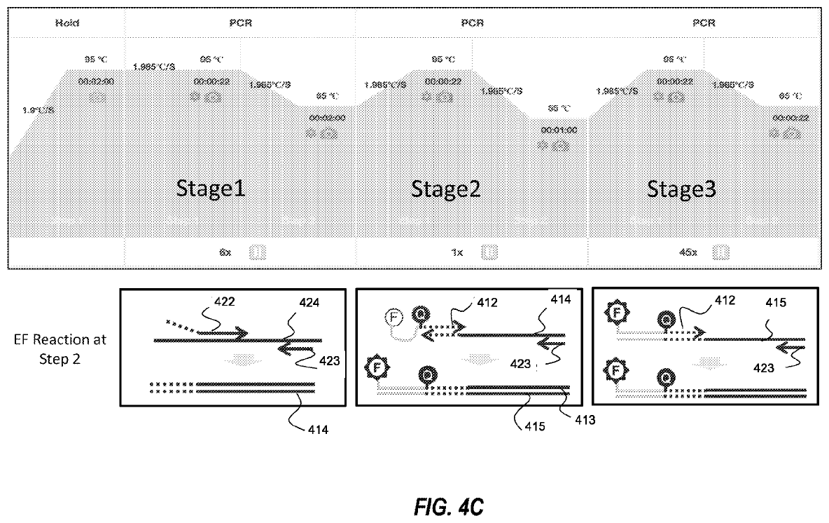

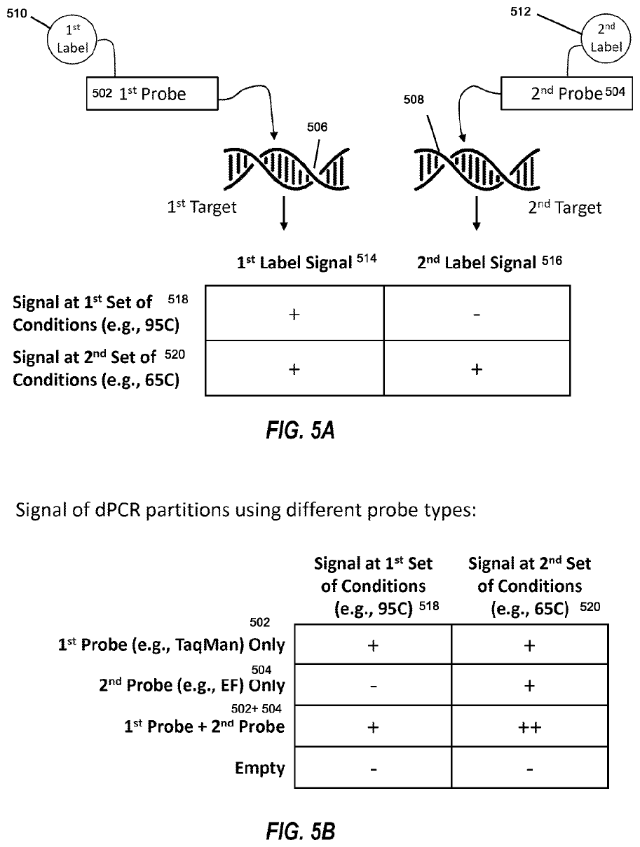

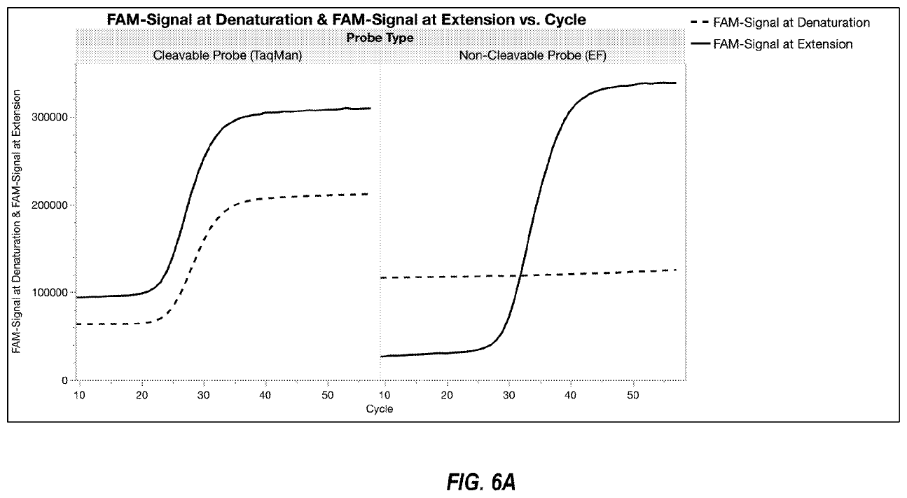

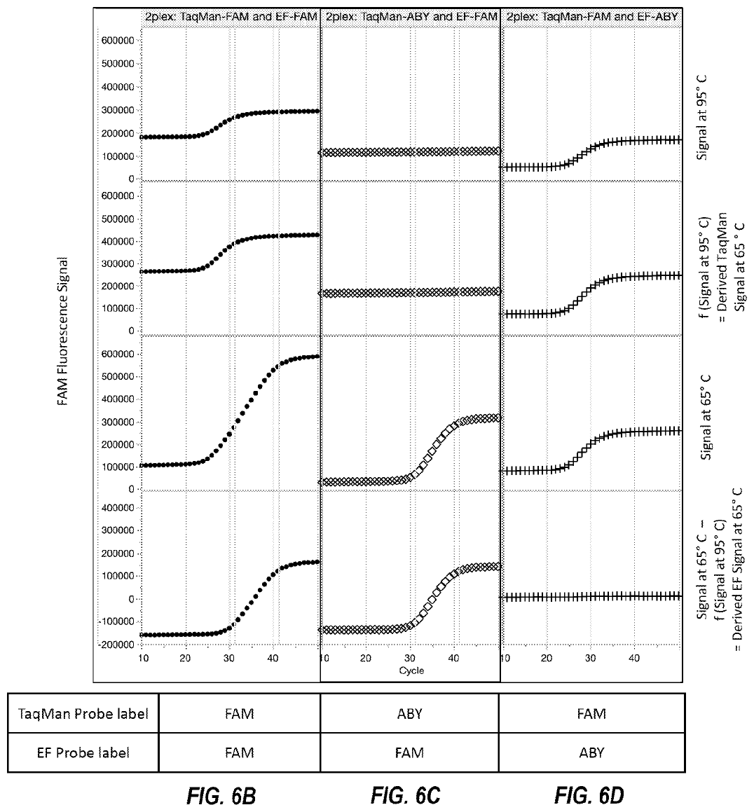

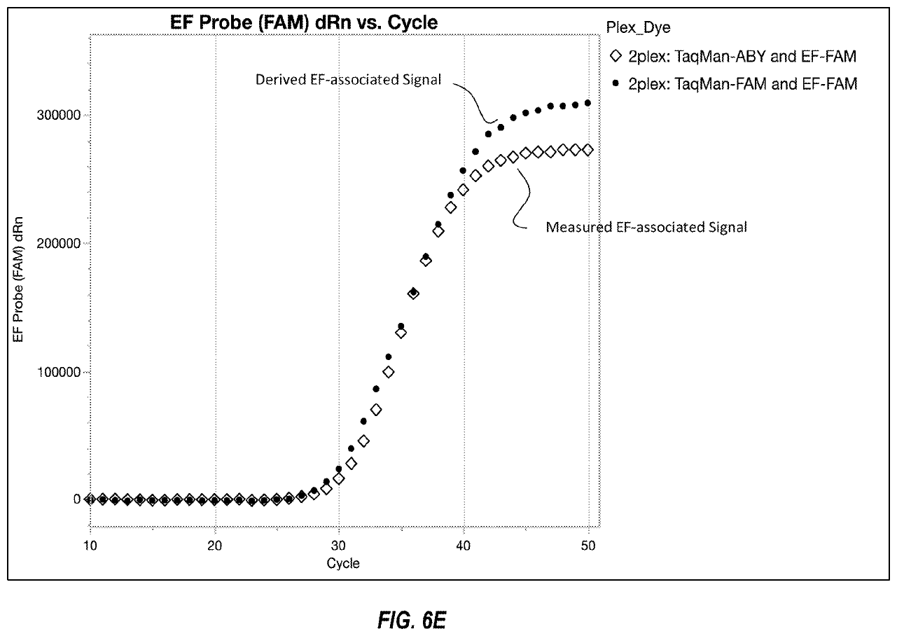

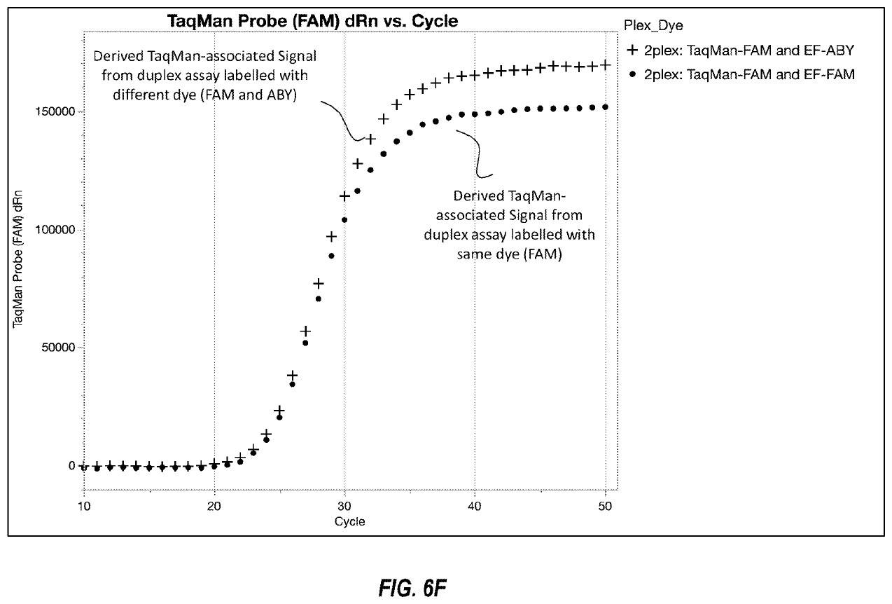

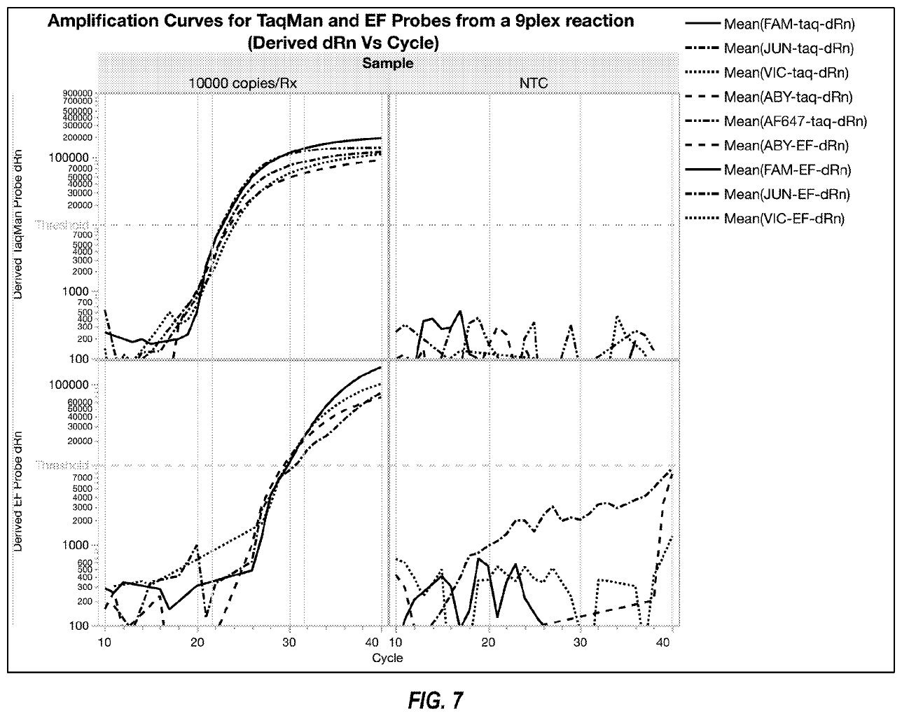

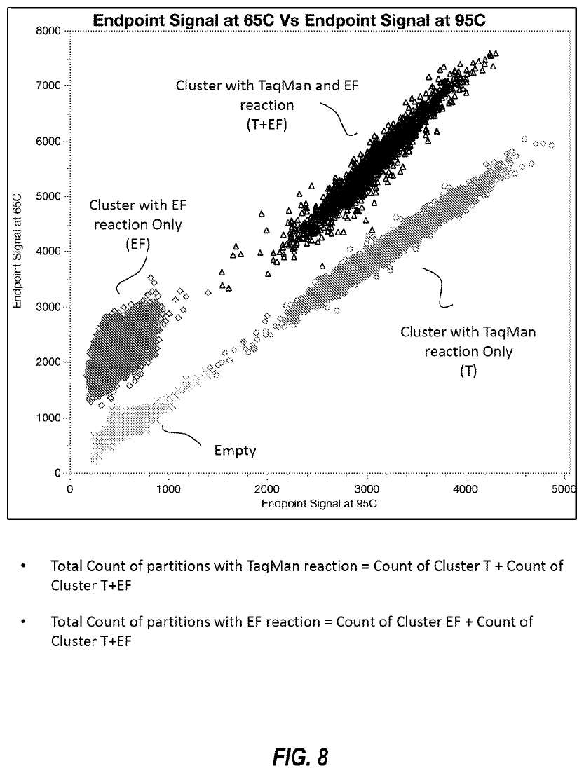

FIG. 2A is a schematic overview of a method for detecting multiple target nucleic acids utilizing detectable labels having spectral similarity by providing different first and second probe types, varying the reaction mixture conditions, and measuring the resulting total signal at each set of conditions. As shown, a first probe 202 is designed to specifically interact (“bind”) with a first target 206. The first probe 202 includes a first label 210 that can generate a first label signal 214. A second probe 204 is designed to specifically interact with a second target 208 that is different from the first target 206. The second probe 204 includes a second label 212 that can generate a second label signal 216.

In some embodiments, the first and second labels 210 and 212 are the same. For example, the first and second labels 210 and 212 may comprise the same fluorescent dye. In some embodiments, the first and second labels 210 and 212 may be different, but are nonetheless designed to generate a substantially identical signal (e.g., have spectral similarity). For example, the first and second labels 210 and 212 may comprise dyes that are chemically distinct yet function to emit fluorescence signals with similar wavelengths. In some embodiments, the first and second label signals 214 and 216 are measured using the same detection channel (e.g., including optical filter arrangement) in the detection instrument.

The first probe 202 and second probe 204 may be provided in the same reaction mixture and allowed to specifically interact with any first and second target 206, 208, respectively, in the reaction mixture. As shown, the reaction mixture is subjected to at least two different sets of reaction conditions. The first probe 202 is designed such that the first label 210 generates the first label signal 214, to a degree correlated with (e.g., proportional to) the amount of specific interaction between the first probe 202 and first target 206, during both the first and second sets of conditions 218 and 220. In contrast, the second probe 204 is designed such that the second label 212 generates the second label signal 216, to a degree correlated with (e.g., proportional to) the amount of specific interaction between the second probe 204 and second target 208, during the second set of conditions 220 but not during the first set of conditions 218. Stated differently, under the first set of conditions 218, the first label signal 214 is increased as a result of specific interaction of the first probe 202 with the first target 206, but the second label signal 216 is not emitted (increased) as a result of specific interaction of the second probe 204 with the second target 208. Under the second set of conditions 220, the second label signal 216 is increased as a result of specific interaction of the second probe 204 with the second target 208, while the first label signal 214 also is further increased or remains at the increased level to at least some degree from the first set of conditions 218.

During the first set of conditions 218, the second label 212 will not generate “substantial signal (e.g., fluorescence),” and the second label signal 216 will therefore not be substantially different from a background (i.e., baseline) level of emission signal (e.g., fluorescence) in the reaction mixture. That is, while there may be some non-zero level of signal generated by the second label 212 during the first set of conditions 218, the second label signal 216 will typically remain below a threshold value that separates background signal from meaningful signal. This threshold may vary according to particular testing protocols and application needs, as discussed above.

In at least some embodiments, when both the first and the second targets 206 and 208 are present in the reaction mixture, the second label signal 216 will differ between the first and second sets of conditions 218 and 220 to a greater degree than the first label signal 214 will differ between the first and second sets of conditions 218 and 220. Thus, while the first label signal 214 may differ somewhat between the first and second sets of conditions 218 and 220, this difference will typically be less than the difference in the second label signal 216 between the first and second sets of conditions 218 and 220.

Various embodiments of the present disclosure exploit the difference in the way the first and second label signals 214 and 216 respond to the different sets of conditions so as to enable the detected first and second label signals 214 and 216 to be resolved (separated), even, for example, if they are detected within the same detection channel. As illustrated, The total signal during the first set of conditions 218 (“the first total signal”) is measured and the total signal during the second set of conditions 220 (“the second total signal”) is measured. Fluorescence (or emission) signal data representing the first total signal is sometimes referred to herein as “first fluorescence signal data” or “first emission signal data”, and fluorescence signal data representing the second total signal is sometimes referred to herein as “second fluorescence signal data” or “composite fluorescence signal data” or “second emission signal data” or composite emission signal data.” As used herein, first and second in this context is not necessarily used to denote a temporal order of detection or the conditions, although such temporal order may occur.

During the first set of conditions 218, the total signal will be substantially equal to the first label signal 214. That is, the first total signal is primarily composed of the first label signal 214, whereas contribution from the second label signal 216 is negligible. During the second set of conditions 220, the total signal will include a combination of the first and second label signals 214 and 216. The first and second label signals 214 and 216 can therefore be separately resolved based on the first and second total signals. For example, the first label signal 214 can be determined based on the first total signal, and the second label signal 216 can be resolved by subtracting the first total signal from the second total signal.

In some embodiments, the first label signal 214 is equated directly to the first total signal. In other embodiments, the first label signal 214 is determined as a function of the first total signal. In some embodiments, this function is a linear function (though non-linear functions may be used in some implementations). For example, as discussed above, the first label signal 214 may differ slightly between the first and second sets of conditions 218, 220 even when the amount of first target 206 has not changed. In certain applications, the first label signal 214 under the second set of conditions 220 may better correspond to standard curves that equate the first label signal 214 to first target 206 amounts. Estimating the first label signal 214 as a function of the first total signal, rather than as directly equal to the first total signal, can therefore bring the calculated first label signal 214 closer to what would be measured under the second set of conditions 220 (i.e., without any interfering second label signal 216).

In some embodiments, the function for converting the first total signal to the first label signal 214 is determined by comparing, in the absence of any second probe interacting with second target, the first label signal 214 under the first set of conditions 218 to the first label signal 214 under the second set of conditions 220. The first label signal 214 under the first set of conditions 218 and under the second set of conditions 220 often correlate to one another according to a linear function. In other embodiments, they can be correlated using non-linear functions. When a linear function is used, a multiplier factor (e.g., correction factor) can be used to convert the first total signal to the first label signal 214. Once such a linear function is determined, it can be used in subsequent assays without necessarily requiring additional comparisons of the first label signal 214 under the first set of conditions 218 and under the second set of conditions 220 in the absence of second probe with second target. In some embodiments, the function for converting the first total signal to the first label signal may be non-linear. In some embodiments, the function/correlation is determined over stages of a thermal cycle or between thermal cycles at which the number of cleaved probes is expected to be the same. This approach can be used to resolve the different signals even if detected within the same detection channel, for example.

As described in greater detail below, the first probe 202 and the second probe 204 have different mechanisms of action that enable different signal responses, depending on the probe type, to the first and second sets of conditions 218 and 220. Beneficially, the ability to resolve the separate signals respectively associated with each of the different probe types need not rely on attributes such as different melting temperatures of the probes. Thus, although the first probe 202 and second probe 204 may have dissimilar melting temperatures, that is not a prerequisite to allow their associated label signals to be effectively resolved. In some embodiments, for example, a melting temperature (Tₘ) (generally defined as the temperature at which 50% of the strands are in double-stranded form and 50% are single-stranded) of the first probe 202 and a Tₘ of the second probe 204 are within about 8° C., or about 6° C., or about 4° C., or about 2° C. of each other. In some embodiments, both probes are bound (e.g., hybridized) to their respective targets under the first or second set of conditions. Optionally, both probes are not substantially bound (e.g., hybridized) to their respective targets under the first or second set of conditions. In one example, both the first and second probes are substantially bound (e.g., hybridized) to their respective targets under the first set of conditions, whereas both the first and second probes are not substantially bound (e.g., hybridized) to their respective targets under the second set of conditions. In another example, both the first and second probes are not substantially bound (e.g., hybridized) to their respective targets under the first set of conditions, whereas both the first and second probes are substantially bound (e.g., hybridized) to their respective targets under the second set of conditions.

FIG. 2B is a graph showing signal response over time for the method outlined in FIG. 2A when the reaction mixture is cycled between the first set of reaction conditions 218 and the second set of reaction conditions 220 and when both the first and second targets 206, 208 are present in the reaction mixture. The cycling of conditions may comprise, for example, differing conditions of various stages associated with thermal cycling in a nucleic acid amplification reaction, such as PCR for example. Under such a reaction, the first set of reaction conditions 218 can correspond to supporting a denaturation stage and the second set of reaction conditions 220 can correspond to supporting an annealing and/or extension stage (“annealing/extension stage”) of the thermal cycling. Thus, in various embodiments, the first set of reaction conditions 218 includes a first temperature or first range of temperatures and the second set of reaction conditions 220 includes a second temperature or second range of temperatures (lower than the first).

As shown, both the first label signal 214 and the second label signal 216 increase under the second set of reaction conditions 220. Under the first set of reaction conditions 218, the first label signal 214 remains roughly the same as at the end of the previous cycle (though it may vary slightly, as discussed above), whereas the second label signal 216 drops to a level similar to the baseline signal level of the second label signal 216, which baseline signal level can be substantially constant over multiple amplification cycles. In other words, the second label signal 216 exhibits a baseline signal above the background signal level during the first set of reaction conditions. In some cases, the second label signal can exhibit a base line signal level that changes at differing stages of an amplification cycle, but nevertheless is sufficiently distinguishable from and lower than the level under the second set of reaction conditions. This may be due to a different state of the probe and proximity of a quencher to the label.

As shown, both the first label signal 214 and the second label signal 216 cumulatively increase at each successive occurrence of the second set of conditions 220. This is a result of additional specific interaction in the reaction mixture between the first probe 202 and the first target 206 and additional specific interaction in the reaction mixture between the second probe 204 and the second target 208. However, where the first label signal 214 remains at a similar level when moving from the end of one cycle to the beginning of another (i.e., when moving from the second set of conditions 220 at the end of a cycle to the first set of conditions 218 at the beginning of a subsequent cycle), the second label signal 216 returns to a level near baseline at the beginning of each cycle (i.e., at each occurrence of the first set of conditions 218).

While some embodiments described herein can be utilized using intra-channel multiplexing (detection within a same channel), the disclosure is not limited to such. Moreover, the present disclosure also contemplates the inter-channel multiplexing and intra-channel multiplexing combined with inter-channel multiplexing to further increase the plexy of the assay. For example, an assay may be designed with multiple different dyes (and thus with multiple different detection channels), where two or more of the different channels each include multiple detectable signals that can be resolved using the techniques and methods described herein.

### Cleavable & Non-Cleavable Probes

In some embodiments, the first probe (e.g., first probe 202) is a “cleavable” probe. The first probe may be designed such that the first label (e.g., first label 210) is detached from the first probe (and released from a corresponding quencher, for example) as a result of hybridization of the first probe to the first target (e.g., first target 206). Once released, the first label therefore continues to contribute to the total signal in the reaction mixture. The first probe may be a TaqMan probe, for example, which undergoes cleavage as a result of 5′ to 3′ exonuclease activity of DNA polymerase during extension of the target molecule to which the probe is hybridized. TaqMan probes are described in U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and 7,445,900, all of which are hereby incorporated herein by reference.

In some embodiments, the second probe (e.g., second probe 204) is a “non-cleavable” probe. The second label (e.g., second label 212) of a non-cleavable probe is intended to remain associated with the probe throughout the assay, and to vary in the level of generated signal (e.g., second label signal 216) according to probe configuration rather than release of the label. The second probe may be an extendable fluorogenic (“EF”) probe, for example, which quenches the label when in a single-stranded configuration but allows signal when incorporated into a double-stranded molecule.

In some embodiments, the second probe (e.g., second probe 204) is a compound or salt thereof as described below, and in the patent application entitled “Multiplex Dye Compounds” filed concurrently in the U.S. Patent and Trademark Office on Jun. 29, 2023, the entire contents of which are incorporated herein by reference.

FIG. 3A illustrates activity of a cleavable probe 302, which in various embodiments can be a TaqMan probe, and a non-cleavable probe 312, which in various embodiments can be an EF probe, during annealing, extension, and denaturation stages of a PCR reaction thermal cycle. As shown, the TaqMan probe 302 hybridizes to its corresponding target nucleic acid amplicon 304 (as used herein target nucleic acid amplicon can refer to a single strand of the target double-stranded nucleic acid and should be understood by reference to the context when describing a PCR reaction) during the annealing stage. During extension of a primer 303 hybridized to the target nucleic acid amplicon 304 upstream of the probe 302, the 5′ to 3′ exonuclease activity of a DNA polymerase cleaves the TaqMan probe label 306 from the remainder of the probe 302, thereby separating it from the corresponding TaqMan probe quencher 309. This leads to a corresponding increase in the fluorescence signal. During denaturation, the label 306 remains free within the reaction mixture solution and thus continues to contribute to the total fluorescence signal.

The EF probe 312 includes an EF probe label 316 and an EF probe quencher 319 which remain in proximity to one another while the probe 312 is in a single-stranded configuration. The fluorescence signal from the label 316 thus remains substantially quenched while the EF probe is in a single-stranded configuration. During the annealing and extension stages, the EF probe 312 hybridizes to its corresponding target template amplicon 314 and is extended to form an extended probe amplicon 313. Extension of target template 314 then forms the complement 315 of the extended probe amplicon 313. The resulting double-stranded amplicon 317 forces the label 316 away from the quencher 319 to a distance sufficient to allow fluorescence emission. During denaturation, the extended probe amplicon 313 is separated from its complement 315. When returned to the single-stranded configuration, the label 316 and quencher 318 are brought back into proximity and fluorescence is again quenched.

FIG. 3B is a graph showing the fluorescence signals from the TaqMan probes 302 and the EF probes 312 over time during thermal cycling of an amplification process. The temperatures of the thermal cycling may be varied according to particular application needs. As an example, the denaturation stage may be carried out at a temperature in a range of from about 80° C. to about 100° C., for example about 85° C. to about 95° C., or for example from about 90° C. to about 95° C.r. The annealing/extension stage may be carried out at a lower temperature, such as in a range from about 40° C. to about 75° C., for example from about 50° C. to about 70° C., for example from about 55° C. to about 65° C. In some implementations, the first set of reaction conditions (e.g., first set of conditions 218, as discussed with reference to FIG. 2A) corresponds to a denaturation stage 318, while the second set of reaction conditions (e.g., second set of conditions 220, as discussed with reference to FIG. 2A) corresponds to an annealing/extension stage 320.

While various embodiments cycle between a denaturation stage 318 and a combined annealing/extension stage 320 (i.e., the amplification process cycles between two target temperatures), other embodiments may include separate annealing and extension steps. In such embodiments, the temperature, and possibly other reaction conditions, may be varied between the annealing and the extension steps. For example, the extension step may be carried out at a higher temperature than the annealing temperature. In some embodiments, the amplification process cycles between at least two target temperatures for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the cycles of the amplification process.

FIG. 3B shows that the fluorescence signal associated with the TaqMan probe 302 increases during the extension stage 320 and then remains at a similar level through the denaturation stage 318 of the next cycle, whereas the fluorescence signal associated with the EF probe 304 increases during the extension stage 320 but decreases to the baseline signal level associated with the EF probe 304 once the subsequent denaturation stage 318 reaches the target denaturation temperature. Those having ordinary skill in the art would appreciate that the cycles N, N+1, N+2 of FIG. 3B may begin at a different stage, however, in which case the comparison of signal levels noted above may be shifted.

In some embodiments, the first set of reaction conditions (e.g., the denaturation conditions 318) comprises a first measurement temperature at which the first label signal is measured, and the second set of reaction conditions (e.g., the annealing/extension conditions 320) comprises a second, different measurement temperature at which the first and second label signal is measured. In some embodiments, the first and second measurement temperatures differ by at least about 10° C. or more, about 15° C. or more, about 20° C. or more, about 25° C. or more, or about 30° C. or more. The first measurement temperature may be the target denaturation temperature in a range of, for example, about 80° C. to about 100° C., for example about 85° C. to about 95° C., or for example from about 90° C. to about 95° C., and the second measurement temperature may be the target annealing/extension temperature in a range of, for example, from about 40° C. to about 75° C., for example, from about 50° C. to about 70° C., for example from about 55° C. to about 65° C.

### EF Probe Template Formation

FIG. 4A illustrates a process of using a primer with a tail (also referred to herein as tailed primer) 422, which is specific to a nucleic acid target 424, to form the target template 414 to which the EF probe 412 can hybridize. The tailed primer 422 includes a tail 426 and a target-specific portion 428. FIG. 4B illustrates an example of the tailed primer 422 as a forward primer, a target specific primer 423 paired with the tailed primer 422 as a reverse primer, and a more detailed view of the EF probe 412.

As shown, in a first stage, the target-specific portion 428 hybridizes to the target 424. Extension of the target-specific portion 428 forms a tailed amplicon 425. Primer 423, which is paired with the tailed primer 422, enables extension of the complement of the tailed amplicon 425. It is this complement that forms the target template 414. As shown, the target template 414 includes a tail complement portion 427.

In a second stage, the EF probe 412 hybridizes to the target template 414 and amplification can continue as shown in FIG. 3A. As shown in FIG. 4A, the EF probe 412 includes a probe tail 417 that has substantial homology with the tail 426 and is therefore complementary to the tail complement portion 427 of the target template 414. Extension of probe 412 and target template 414 forms the double-stranded amplicon 419. In the third stage, the primer 423, shown here paired with the tailed primer 422, may also function as the primer 423 that pairs with the EF probe 412 to enable formation of the double-stranded amplicon 419, as shown in FIG. 3A.

As shown in FIG. 4B, the tail 426 can form the 5′ end of the tailed primer 422. The EF probe 412 can include a stem-loop portion, with stem portions 410 on either side of a loop portion 411, configured to form a stem-loop structure when the EF probe 412 is single-stranded. For example, the label 416 may be located on one side of the stem-loop portion and the quencher 418 may be located on the opposite side of the stem-loop portion such that the label 416 and quencher 418 are brought into proximity when the stem-loop structure is formed but spaced farther apart when the EF probe 412 is constrained into a more linear configuration (e.g., when incorporated into a double-stranded amplicon).

In the illustrated embodiment, the label 416 is located at or near the 5′ end of the EF probe 412 and the quencher 418 is located 3′ of the label 416. The positions of the label 416 and quencher 418 may be reversed in other embodiments. Preferably, as shown, the stem-loop portion is disposed 5′ of the probe tail 417 so that the stem-loop portion remains at the end of the amplicons resulting from extension of the EF probe 412, so that stem-loop structure formation (when single-stranded) is less likely to be compromised.

In some embodiments, the EF probe includes a non-stem-loop portion separating the label (located at or near the 5′ end of the EF probe) from the quencher located at or near the 3′ of the EF probe.

In addition to or alternative to the EF probes described herein, some embodiments may include other labelled oligonucleotides that generate increased fluorescence upon being incorporated into a double-stranded amplicon (relative to when in a single-stranded state), such as, for example during extension and/or annealing stages of a PCR process. For example, LUX™ primers include an internal fluorophore that is quenches by a hairpin structure located 5′ of the fluorophore. As with EF probes, a LUX™ primer provides increased fluorescence when incorporated into a double-stranded amplicon and the hairpin structure is linearized. Further, any of the primers or probes described herein may include one or more locked nucleic acids (LNAs) as are known in the art.

In some embodiments, the tailed primer 422 and the corresponding (non-tailed) primer 423 are provided at different concentrations. For example, the primer 423 may be provided at a higher concentration than the tailed primer 422. For example, the primer 423 may be provided at a concentration that is about 2× (2 times) to about 30× (30 times) the concentration of the tailed primer 422, or about 5× to about 25× the concentration of the tailed primer 422, or about 10× to about 20× the concentration of the tailed primer 422. Because the primer 423 can function to both (1) drive the formation of the target template 414 (as shown in FIG. 4A) and (2) drive the formation of the complement 415 of the extended probe amplicon 413 (as shown in FIG. 3A), providing it at a higher concentration than the corresponding tailed primer 422 can beneficially balance the reaction and help drive overall reaction efficiency.

In some embodiments, the EF probe 412 is provided at a concentration that is different from the concentration of the tailed primer 422 and/or the concentration of the primer 423. For example, the EF probe 412 may be provided at a concentration that is greater than the concentration of the tailed primer 422 and that is less than the concentration of primer 423. In some embodiments, the EF probe 412 is provided at a concentration that is about 2× to about 20× the concentration of the tailed primer 422, or about 3× to about 15× the concentration of the tailed primer 422. As discussed above, providing the primer 423 at a relatively higher concentration helps to drive the overall efficiency of the reaction. Providing the EF probe 412 at a concentration that is higher than the tailed primer 422, but not necessarily higher than the primer 423, pushes more of the associated amplification toward the EF probe 412 as opposed to the tailed primer 422, yet still allows the primer 423 to function as the primary driver of reaction efficiency.

In addition to or alternative to the “universal” EF probes that use a probe tail 417, other embodiments include and/or utilize EF probes with a target-specific portion rather than a probe tail 417. Such EF probes can directly hybridize to a target template nucleic acid as shown in FIG. 4A and therefore do not need to follow the two-stage process shown in FIG. 4A for generating a target template 414 with a tail complement portion 427. In such embodiments, the probe tail 417 of the EF probe 412 is replaced with a target-specific portion that directly hybridizes to the target 424. The process is otherwise similar to that shown in FIG. 3A. That is, after the EF probe is extended, a subsequent round of annealing/extension will extend the complement strand, forming the double-stranded amplicon that separates the fluorophore and the quencher to allow for fluorescence signal generation.

FIG. 4C illustrates a three-stage thermal cycling method that may be utilized during an amplification process involving non-cleavable probes (e.g., EF probes) and optionally cleavable probes (e.g., TaqMan probes). The amplification process shown in FIG. 4C may be used in conjunction with any of the other methods disclosed herein. The illustrated amplification process includes a first stage with a first target annealing/extension temperature, a second stage with a second, different annealing/extension temperature, and a third stage with a third annealing/extension temperature. In this embodiment, the third annealing/extension temperature is the same as the first annealing/extension temperature. Other embodiments may include a third annealing/extension temperature that is different from both the first and second annealing/extension temperatures.

The illustrated amplification process thus includes a denaturation temperature and multiple different annealing/extension temperatures that vary throughout the amplification process. Such an amplification process beneficially provides an initial stage (Stage 1) in which target template 414 is primarily formed, an intermediate stage (Stage 2) in which there is increased interaction between EF probes 412 and the target templates 414 to form the initial extended probe amplicons 413, and a later stage (Stage 3) in which amplification further involving the probe amplicons 413 and 415 can proceed.

As shown, the first annealing/extension temperature may be higher than the second annealing/extension temperature. The first series of denaturation and annealing/extension steps (in Stage 1) are cycled a greater number of times than the second series of denaturation and annealing/extension steps (in Stage 2). The third series of denaturation and annealing/extension steps (in Stage 3) may be cycled a greater number of times than the first series of denaturation and annealing/extension steps. The denaturation temperature may be the same for each stage or can differ. By way of non-limiting example, the denaturation temperature the various stages could be more than 80° C., but differ from each other. For example, the temperature at an earlier could be higher than in a subsequent stage. In an embodiment, the denaturation temperature in the first stage could be about 95° C. and in the second stage could be about 85° C..

Stages 1 and 2 thus function as pre-loading stages that primarily generate target template 414 (in Stage 1) and then provide a lower annealing/extension temperature (in Stage 2), for at least one cycle, to allow increased interaction between the EF probes 412 and the target templates 414. Afterwards, multiple amplification cycles can then be carried out at the third annealing/extension temperature to drive amplification primarily involving the EF probes 412, primer 423 and/or their extended probe amplicons 413 and 415. Most of the amplification cycles are thus typically carried out during Stage 3.

The various specific temperatures, times, and rates of temperature change depicted in FIG. 4C are illustrative only and should not be understood as limiting of the scope of the present disclosure and claims. Other specific temperatures, times, and rates of temperature change can be employed and will be understood from the remainder of the disclosure.

Embodiments are not limited to a three-stage thermal cycling method. A one-stage thermal cycling method (where the annealing/extension and denaturation temperatures do not vary from cycle to cycle) or a two-stage thermal cycling method (where one of the annealing/extension temperatures, or the denaturation temperatures, varies from cycle to cycle) may be utilized during an amplification process involving non-cleavable probes (e.g., EF probes) and optionally cleavable probes (e.g., TaqMan probes).

### Digital PCR Embodiments

PCR is an in vitro method for the enzymatic synthesis of specific DNA sequences using two oligonucleotide primers that hybridize to opposite strands and flank the region of interest in target DNA. A repetitive series of reaction stages involving template denaturation, primer annealing, and the extension of the annealed primers by DNA polymerase results in the exponential accumulation of a specific fragment whose termini are defined by the primers. PCR can selectively enrich a specific DNA sequence by several orders of magnitude. As discussed above, the annealing and extension stages can be distinct stages at differing reaction conditions or can occur under a same set of reaction conditions.

Digital polymerase chain reaction (dPCR) is a specific application of PCR that can be used to directly quantify target nucleic acids in a sample. In dPCR, the reaction mixture is partitioned into many small reaction volumes (also referred to as partitions) so that the target nucleic acid is in some, but not all, of the reaction volumes. The reaction volumes are subjected to thermal cycling, and the proportion of “positive” reaction volumes that generate a signal (e.g., an emission signal, such as fluorescence, from a detectable label) indicative of the presence of the target is determined. Quantitation is based on application of Poisson statistics, using the number of negative/non-reactive reaction volumes and assuming a Poisson distribution to establish the number of initial copies that were distributed across all the reaction volumes.

The features and principles described herein with respect to other amplification processes are also generally applicable to dPCR processes. Thus, although the description of this section provides a more detailed disclosure of dPCR embodiments, the disclosure provided elsewhere herein is also applicable to dPCR embodiments. Embodiments that include dPCR may utilize a variety of partitioning mechanisms or devices as known in the art or as may be developed in the future. For example, some conventional dPCR systems utilize a plurality of droplets encapsulated by an oil phase to form the plurality of partitions/reaction volumes. Other embodiments may utilize an array of microchambers. As example of such a system is the QuantStudio Absolute Q system available from Thermo Fisher Scientific, which uses a microfluidic array plate to perform the compartmentalizing/partitioning of sample and generation of reaction volumes. Those having ordinary skill in the art are familiar with various types of systems for partitioning sample into the small reaction volumes, subjecting those reaction volumes to PCR, and detecting the emission signal from the reaction volumes.

In some dPCR embodiments, the reaction mixture is fully formed prior to partitioning into the plurality of reaction volumes. In alternative embodiments, one or more components of the reaction mixture may be pre-loaded onto or into the reaction volumes. For example, probes and/or primers may be coated onto the walls of microchambers, and the sample and/or other components of the reaction mixture are then added to the microchambers to form a plurality of reaction mixtures in each of the reaction volumes.

FIG. 5A is a schematic overview of a method for detecting multiple target nucleic acids using a PCR process and probes carrying detectable labels having spectral similarity. The PCR process can include real-time/quantitative PCR (qPCR) that typically monitors amplification during the reaction, dPCR typically involves an end-point measurement to count and determine the number of “positive” partitions, and/or end-point PCR. Accordingly, FIG. 5A illustrates that under both the first set of conditions 518 (e.g., denaturation conditions such as about 95° C.) and the second set of conditions 520 (e.g., annealing/extension conditions such as about 65° C.) a first probe 502 associated with a first label 510 and configured to specifically interact with a first target 506 will show as positive (+) at the conclusion of the reaction due to emission from the first label signal 514. On the other hand, a second probe 504 associated with a second label 512 and configured to specifically interact with a second target 508 will show as negative (−) under the first set of conditions 518 due to no emission signal from the second label signal 516, but will show as positive (+) under the second set of conditions 520 due to emission signal from the second label signal 516.

This is similar to the schema illustrated in FIG. 2A, and FIG. 5A shows that the same principles can be applied to end-point measurements such as in dPCR. As described in other embodiments, the first probe 502 may be a cleavable probe such as a TaqMan probe, and the second probe 504 may be a non-cleavable probe such as an EF probe.

FIG. 5B illustrates how the signal (e.g., at an end-point cycle of PCR) for a dPCR reaction volume can vary depending on whether the first probe 502, second probe 504, or both were active within the reaction volume during the reaction. As shown, if only the first probe 502 provides a signal 514, the reaction volume will be positive (+) under both the first and second set of conditions 518, 520. If only the second probe 504 provides a signal 516, the reaction volume will be negative (−) under the first set of conditions 518 and positive (+) under the second set of conditions 520. If both the first and second probes 502, 504 provide a signal, the reaction volume will be positive (+) under the first set of conditions 518 and highly positive (++) (high emission signal due to contributions of both label signal 514, 516) under the second set of conditions 520. Of course, if neither probe types provide a signal, the reaction volume will be negative (−) under either set of conditions 518, 520.

The total count of reaction volumes that are positive for the first probe 502 (and thus estimated as positive for the first target 506) is determined by counting the number of reaction volumes that are positive (+) or highly positive (++) under both sets of conditions 518, 520. The total count of reaction volumes that are positive for the second probe 504 (and thus estimated as positive for the second target 508) is determined by counting (i) the number of reaction volumes that are positive (+) under the second set of conditions 520 but negative (−) under the first set of conditions 518, and adding it to (ii) the number of reaction volumes that are highly positive (++) under the second set of conditions 520. These reaction volume counts may be calculated or estimated by plotting the signal under the first set of conditions 518 at an end-point cycle of PCR against the signal under the second set of conditions 520 at the end-point cycle and identifying clusters. See, for instance, the plot of FIG. 8, described in more detail in the Examples section below. Concentrations of the first and second target 506, 508 in the sample may then be estimated using standard dPCR techniques.

Accordingly, a method for determining the presence of and/or amount of multiple targets using the multiplexing techniques described herein in a dPCR application can comprise: preparing a reaction mixture comprising a first probe type (e.g., TaqMan probe) and a second probe type (e.g., an EF probe), designed to specifically interact with respective first and second nucleic acid targets; loading/partitioning a sample into a plurality of reaction volumes; measuring a signal of the reaction volumes at a first set of reaction conditions (e.g., denaturation conditions such as about 95° C.) during an end-point cycle of PCR; measuring a signal of the reaction volumes at a second set of reaction conditions (e.g., annealing/extension conditions such as about 65° C.) during an end-point cycle of PCR; categorizing the reaction volumes according to measured signal properties at the end-point signal measurements; determining or estimating a count for each probe type (i.e., a count of reaction volumes in which the first probe type was active and a count of reaction volumes in which the second probe type was active) based on the categorized reaction volumes; and determining or estimating the presence and/or amount of the first and second nucleic acid targets based on the counts.

### End-Point PCR Embodiments

The analytical techniques in accordance with various embodiments can also be used when conducting a traditional end-point PCR process, in which the sample is subject to PCR in bulk (or larger reaction volumes not intended to capture a single or no DNA molecules using Poisson statistics), and as those of ordinary skill in the art are familiar with. In such a PCR process, the measurements of signal from the two different probe types can occur at an end-point cycle of PCR and at different reaction conditions (such as, e.g., denaturation and annealing and/or extension conditions as described herein), similar to the approach described above for the dPCR process. However, the label signals detected will follow that outlined in FIG. 2A, with the signals thus indicating the presence or absence of the respective first and second targets. With reference to FIG. 3B, the measured end-point cycle signals under the two different reaction conditions thus may result in the differing levels of signal shown schematically at Cycle N+2 (analogizing that to the end-point cycle). By comparing the different signal levels obtained, using the algorithms set forth in FIG. 2A and further described above, the presence or absence of the first and second target nucleic acids can be determined using detectable labels having overlapping emission signal spectra and that are detectable in a same detection channel.

Accordingly, a method for determining the presence or absence of multiple targets using the intra-channel multiplexing features described herein can also be performed using endpoint PCR (i.e., using bulk or larger reaction volumes that are not of a size that is intended to rely on Poisson statistics and capturing a single molecule of target nucleic acid in the reaction volume as those of ordinary skill in the art are familiar with). The method employed in an end-point PCR application includes: preparing a reaction mixture comprising a first probe type (e.g., TaqMan probe) and a second probe type (e.g., an EF probe), configured to specifically interact with respective first and second nucleic acid targets; subjecting the reaction mixture to an amplification reaction (e.g., PCR); measuring end-point cycle signals of the reaction mixture at a first set of reaction conditions (e.g., denaturation conditions such as about 95° C.); and measuring an end-point signal of the reaction volumes at a second set of reaction conditions (e.g., annealing/extension conditions such as about 65° C.) (notably, for the measurements at end-point, the measuring under the second set of reaction conditions (annealing/extension) will occur before the measuring under the first set of reaction conditions (denaturation); determining a presence or absence the first and/or second nucleic acid targets in the reaction mixture by measuring during the first set of reaction conditions a first total emission (e.g., fluorescence) signal that comprises any first emission (e.g., fluorescence) signal if present, measuring during the second set of reaction conditions a second total emission (e.g., fluorescence) signal comprising any first emission (e.g., fluorescence) signal if present and any second emission (e.g., fluorescence) signal if present, and estimating the first emission (e.g., fluorescence) signal and/or second emission (e.g., fluorescence) signal based on the first and second total emission (e.g., fluorescence) signals.

### Compounds

In an embodiment, the second probe or the non-cleavable probe is a compound, or a salt thereof, having the formula:

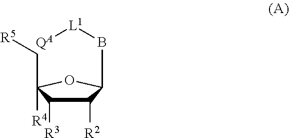

wherein Q^{A} is a quencher moiety,
B is a divalent nucleobase,
L¹ is a divalent linker,
R² is hydrogen or —OR^{2A},
R³ is —OR^{3A} or —O—P(NR^{3B}R^{3C})—OR^{3A},
R⁴ is hydrogen or unsubstituted methyl, or R² and R⁴ substituents are joined to form a substituted or unsubstituted heterocycloalkyl (e.g., 3 to 8 membered, 3 to 6 membered, 4 to 6 membered, 4 to 5 membered, or 5 to 6 membered),
R⁵ is —OR^{5A}, and
R^{2A}, R^{3A}, R^{3B}, R^{3C}, and R^{5A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the quencher moiety is a monovalent form of QSY7.

In embodiments, the quencher moiety is a monovalent form of

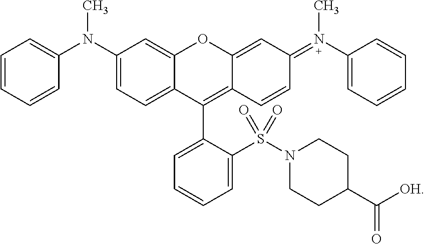

In embodiments, the quencher moiety is

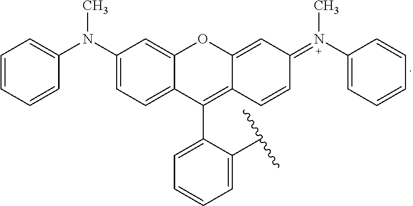

In embodiments, the quencher moiety is a monovalent form of QSY21.

In embodiments, the quencher moiety is a monovalent form of

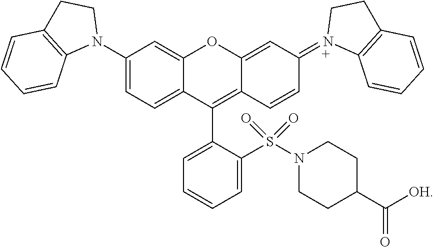

In embodiments, the quencher moiety is

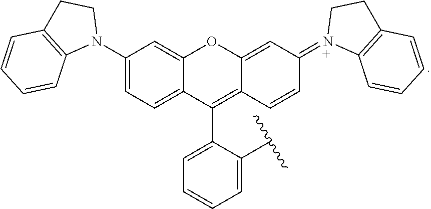

In embodiments, the quencher moiety is a monovalent form of QSY9.

In embodiments, the quencher moiety is a monovalent form of

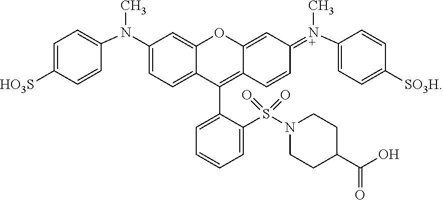

In embodiments, the quencher moiety is

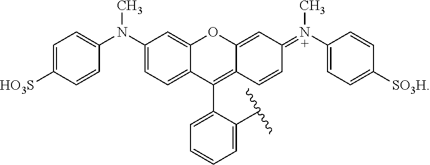

In embodiments, the quencher moiety is a monovalent form of BHQ1.

In embodiments, the quencher moiety is a monovalent form of

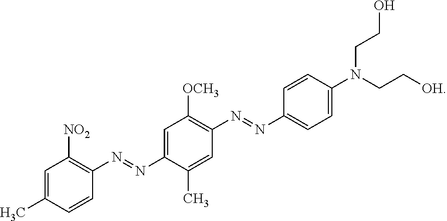 [SMILES: COc1cc(/N=N/c2ccc(C)cc2[N+](=O)[O-])c(C)cc1/N=N/c1ccc(N(CCO)CCO)cc1]

In embodiments, the quencher moiety is

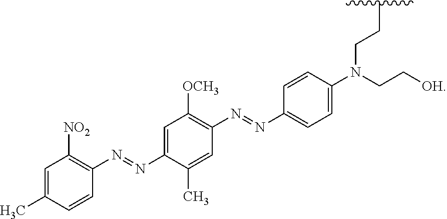 [SMILES: *CCN(CCO)c1ccc(/N=N/c2cc(C)c(/N=N/c3ccc(C)cc3[N+](=O)[O-])cc2OC)cc1]

In embodiments, the quencher moiety is a monovalent form of BHQ2.

In embodiments, the quencher moiety is a monovalent form of

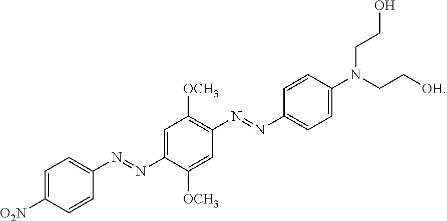 [SMILES: COc1cc(/N=N/c2ccc([N+](=O)[O-])cc2)c(OC)cc1/N=N/c1ccc(N(CCO)CCO)cc1]

In embodiments, the quencher moiety is

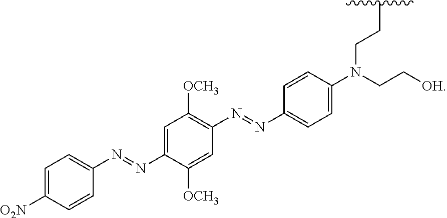 [SMILES: *CCN(CCO)c1ccc(/N=N/c2cc(OC)c(/N=N/c3ccc([N+](=O)[O-])cc3)cc2OC)cc1]

In embodiments, the quencher moiety is a monovalent form of BHQ3.

In embodiments, the quencher moiety is a monovalent form of

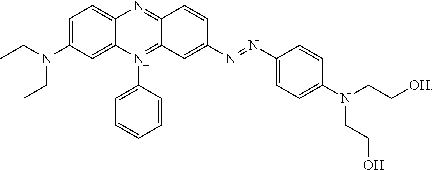

In embodiments, the quencher moiety is

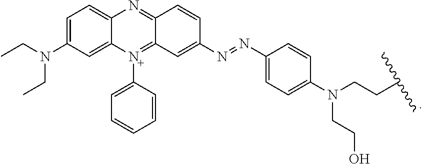

In embodiments, the quencher moiety is a monovalent form of Dabcyl.

In embodiments, the quencher moiety is a monovalent form of

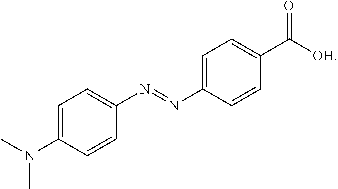 [SMILES: CN(C)c1ccc(/N=N/c2ccc(C(=O)O)cc2)cc1]

In embodiments, the quencher moiety is

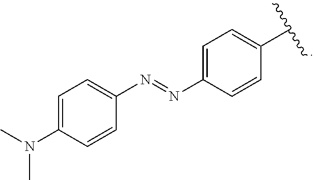 [SMILES: *c1ccc(/N=N/c2ccc(N(C)C)cc2)cc1]

In embodiments, the quencher moiety is a monovalent form of Dabsyl.

In embodiments, the quencher moiety is a monovalent form of

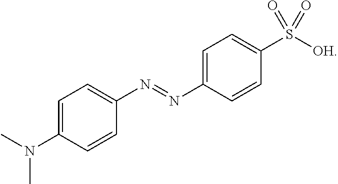 [SMILES: CN(C)c1ccc(/N=N/c2ccc(S(=O)(=O)O)cc2)cc1]

In embodiments, the quencher moiety is a monovalent form of Eclipse.

In embodiments, the quencher moiety is a monovalent form of

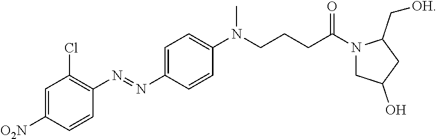 [SMILES: CN(CCCC(=O)N1CC(O)CC1CO)c1ccc(/N=N/c2ccc([N+](=O)[O-])cc2Cl)cc1]

In embodiments, the quencher moiety is

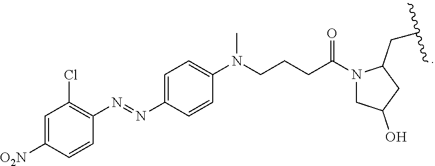 [SMILES: *CC1CC(O)CN1C(=O)CCCN(C)c1ccc(/N=N/c2ccc([N+](=O)[O-])cc2Cl)cc1]

In embodiments, the quencher moiety is a monovalent form of BBQ-650.

In embodiments, the quencher moiety is

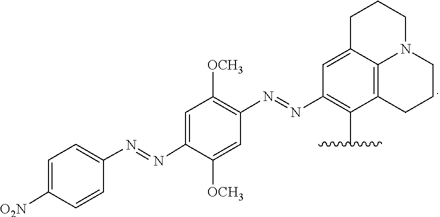 [SMILES: *c1c(/N=N/c2cc(OC)c(/N=N/c3ccc([N+](=O)[O-])cc3)cc2OC)cc2c3c1CCCN3CCC2]

In embodiments, the quencher moiety is a monovalent form of Iowa Black RQ.

In embodiments, the quencher moiety is a monovalent form of Iowa Black FQ.

In embodiments, the quencher moieties above are all interchangeable. In embodiments, the quencher moiety can be substituted in Formulae (I), (IA), (II), (III), (IV), (V), (VI), (VI-1), (VI-2), (VI-3), (VI-4), (VI-5), (VII), (VII-1), (VII-2), (VII-3), (VII-4), (VII-5), (VIII), (VIII-1), (VIII-2), (VIII-3), (VIII-4), (VIII-5), (IX), (IX-1), (IX-2), (JX-3), (JX-4), (JX-5), (X), (XI), (XII), (XIII), (XIV), (XV), (XV-1), (XV-2), (XV-3), (XV-4), (XV-5), (XVI), (XVI-1), (XVI-2), (XVI-3), (XVI-4), (XVI-5), (XVII), (XVII-1), (XVII-2), (XVII-3), (XVII-4), (XVII-5), (XVIII), (XVIII-1), (XVIII-2), (XVIII-3), (XVIII-4), (XVIII-5), and (XIX), and embodiments thereof.

In embodiments, the second probe or the non-cleavable probe is a compound, or a salt thereof, having the formula:

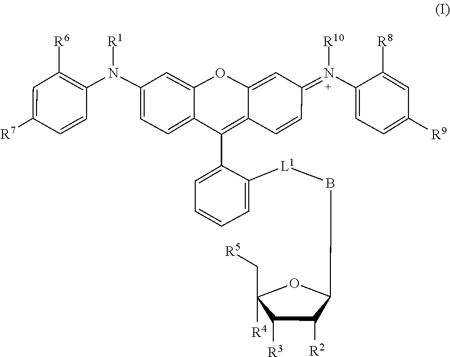

wherein
B is a divalent nucleobase,
L¹ is a divalent linker,
R² is hydrogen or —OR^{2A};
R³ is —OR^{3A} or —O—P(NR^{3B}R^{3C})—OR^{3A}
R⁴ is hydrogen or unsubstituted methyl, or R² and R⁴ substituents are joined to form a substituted or unsubstituted heterocycloalkyl (e.g., 3 to 8 membered, 3 to 6 membered, 4 to 6 membered, 4 to 5 membered, or 5 to 6 membered),
R⁵ is —OR^{5A},
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered), and
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, □NHNH₂, □ONH₂, □NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N—, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ and R⁶ may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R⁸ and R¹⁰ may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{2A}, R^{3A}, R³, R^{3C}, R^{5A}, and R^{A} may independently be hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₈), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

A person having ordinary skill in the art would understand that the compound may exist as a neutral species with a counterion.

In embodiments, the compound has the formula:

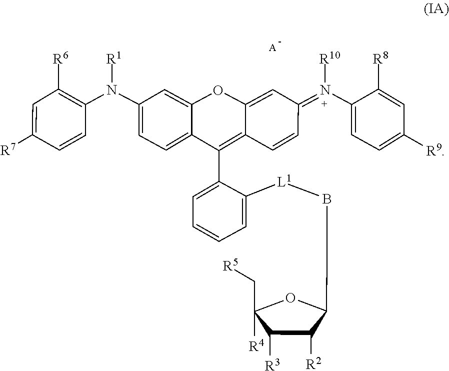

B, L¹, R², R³, and R⁵ are as described herein, including in embodiments. A⁻ is a counterion. In embodiments, A⁻ is Cl⁻. In embodiments, A⁻ is F₃CC(O)O⁻. In embodiments, A⁻ is acetate or bromide.

In embodiments, the compound has the formula:

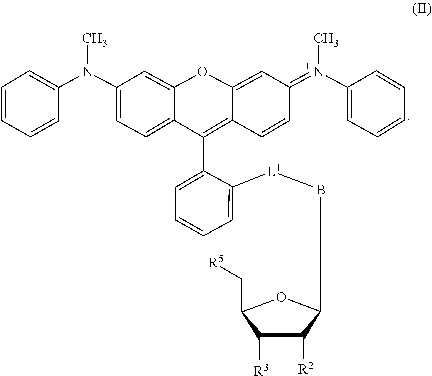

B, L¹, R², R³, and R⁵ are as described herein, including in embodiments.

In embodiments, the compound has the formula:

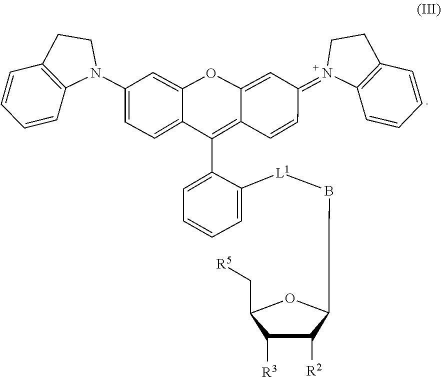

B, L¹, R², R³, and R⁵ are as described herein, including in embodiments.

In embodiments, the compound has the formula:

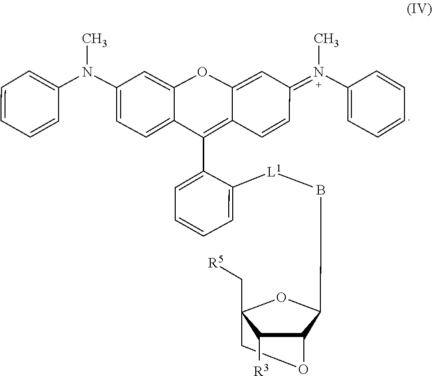

B, L¹, R³, and R⁵ are as described herein, including in embodiments.

In embodiments, the compound has the formula:

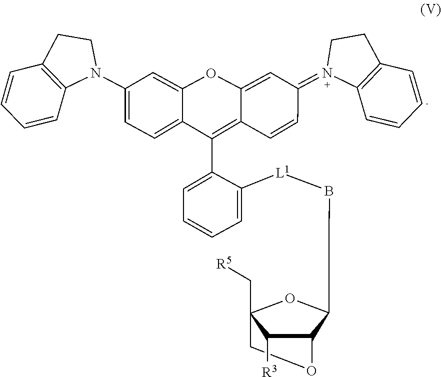

B, L¹, R³, and R⁵ are as described herein, including in embodiments.

In embodiments, B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. In embodiments, B is a divalent cytosine or a derivative thereof, divalent guanine or a derivative thereof, divalent adenine or a derivative thereof, divalent thymine or a derivative thereof, or divalent uracil or a derivative thereof. In embodiments, B is a divalent cytosine or a derivative thereof. In embodiments, B is a divalent guanine or a derivative thereof. In embodiments, B is a divalent adenine or a derivative thereof. In embodiments, B is a divalent thymine or a derivative thereof. In embodiments, B is a divalent uracil or a derivative thereof.

In embodiments, the compound has the formula:

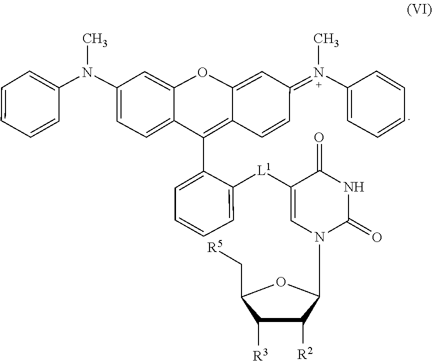

L¹, R², R³, and R⁵ are as described herein, including in embodiments.

In embodiments, the compound has the formula:

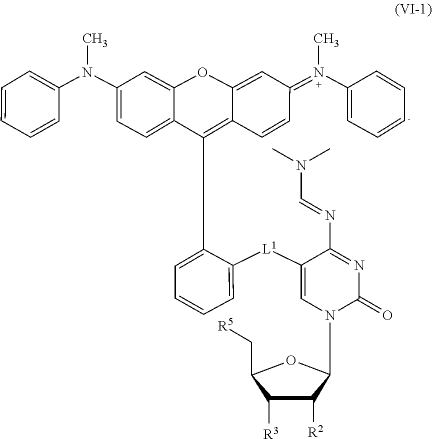

L¹, R², R³, and R⁵ are as described herein, including in embodiments.

In embodiments, the compound has the formula:

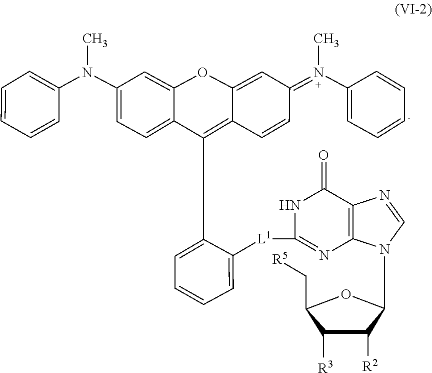

L¹, R², R³, and R⁵ are as described herein, including in embodiments.

In embodiments, the compound has the formula:

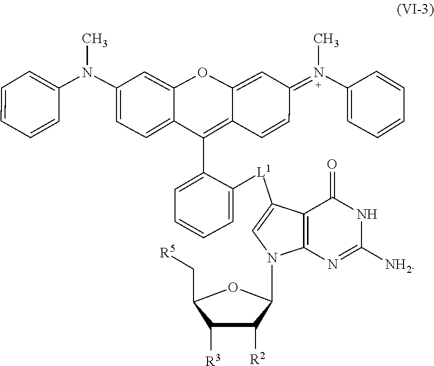

L¹, R², R³, and R⁵ are as described herein, including in embodiments.

In embodiments, the compound has the formula:

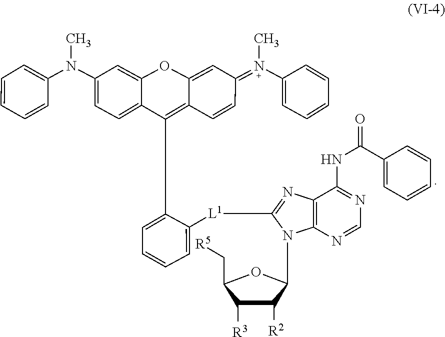

L¹, R², R³, and R⁵ are as described herein, including in embodiments.

In embodiments, the compound has the formula:

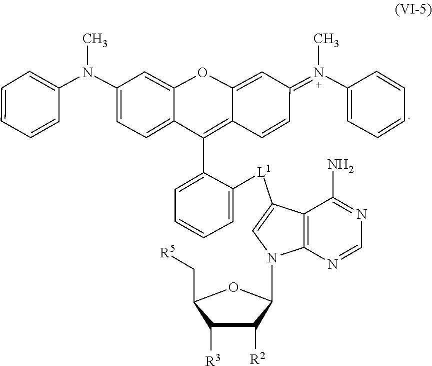

L¹, R², R³, and R⁵ are as described herein, including in embodiments.

In embodiments, the compound has the formula:

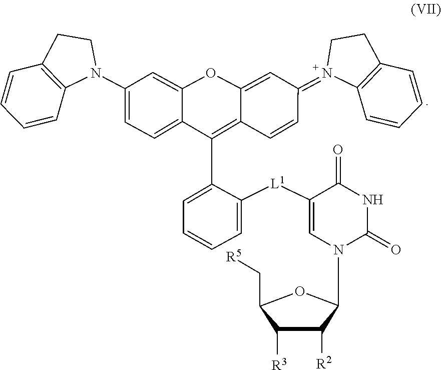

L¹, R², R³, and R⁵ are as described herein: including in embodiments.

In embodiments, the compound has the formula:

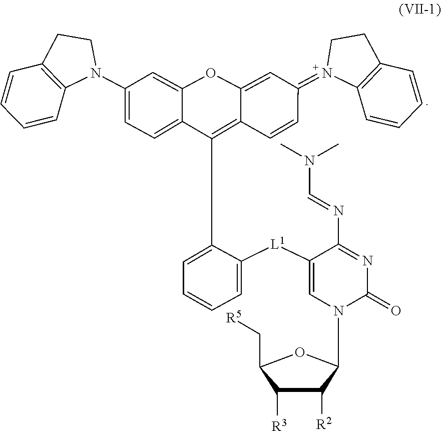

L¹, R², R³, and R⁵ are as described herein, including in embodiments.

In embodiments, the compound has the formula:

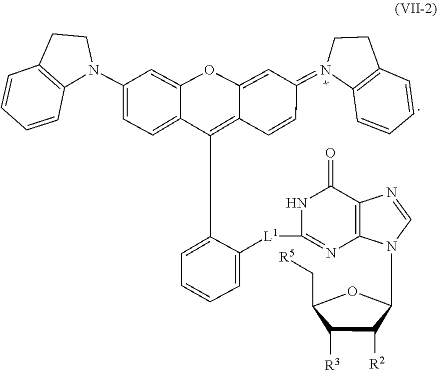

L¹, R², R³, and R⁵ are as described herein, including in embodiments.

In embodiments, the compound has the formula:

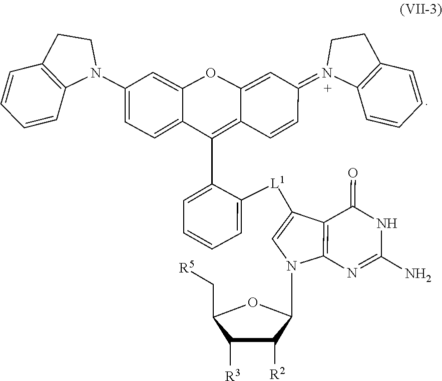

L¹, R², R³, and R⁵ are as described herein, including in embodiments.

In embodiments, the compound has the formula:

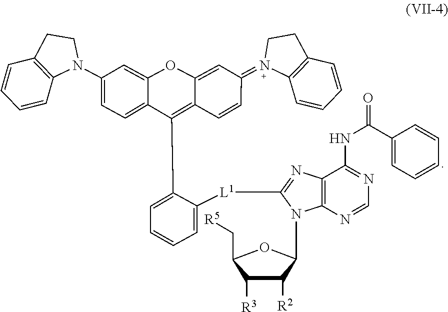

L¹, R², R³, and R⁵ are as described herein, including in embodiments.

In embodiments, the compound has the formula:

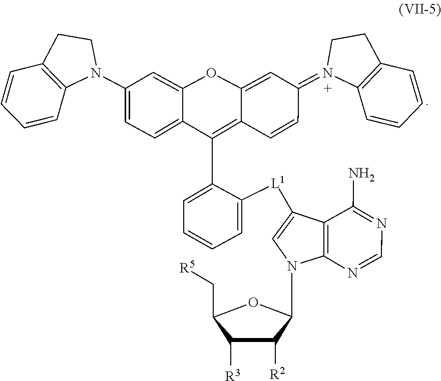

L¹, R², R³, and R⁵ are as described herein, including in embodiments.

In embodiments, the compound has the formula:

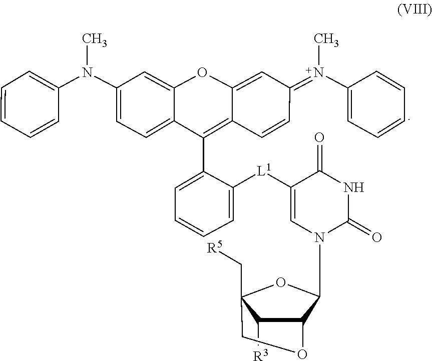

L¹, R³, and R⁵ are as described herein, including in embodiments.

In embodiments, the compound has the formula:

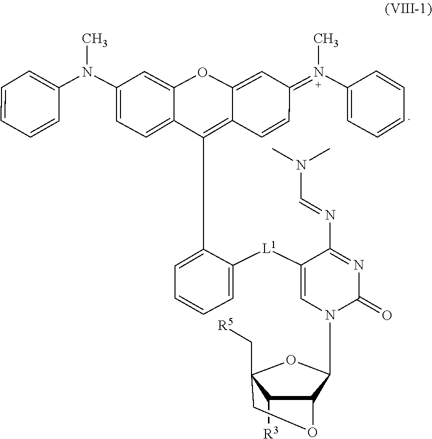

L¹, R³, and R⁵ are as described herein, including in embodiments.

In embodiments, the compound has the formula:

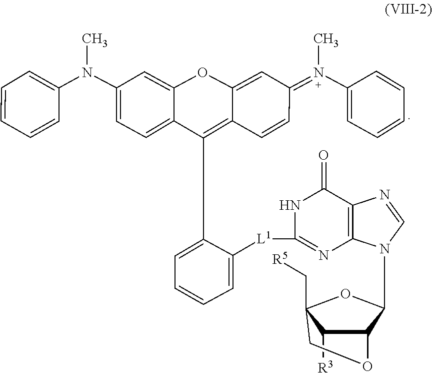

L¹, R³, and R⁵ are as described herein, including in embodiments.

In embodiments, the compound has the formula:

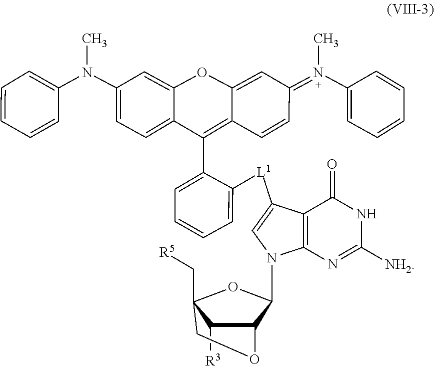

L¹, R³, and R⁵ are as described herein, including in embodiments.

In embodiments, the compound has the formula:

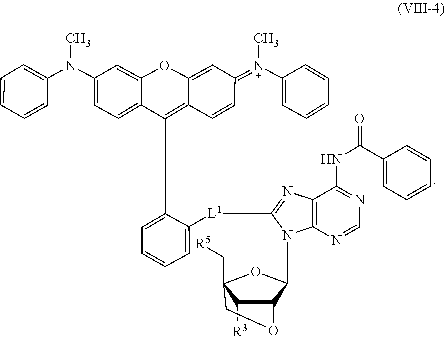

L¹, R³, and R⁵ are as described herein, including in embodiments.

In embodiments, the compound has the formula:

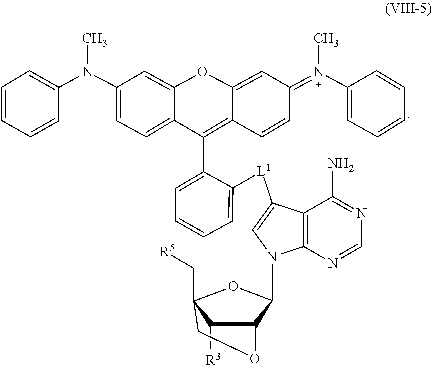

L¹, R³, and R⁵ are as described herein, including in embodiments.

In embodiments, the compound has the formula:

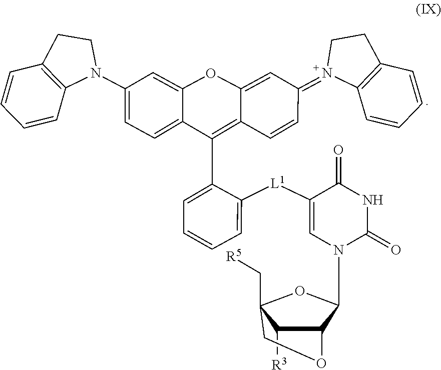

L¹, R³, and R⁵ are as described herein, including in embodiments.

In embodiments, the compound has the formula:

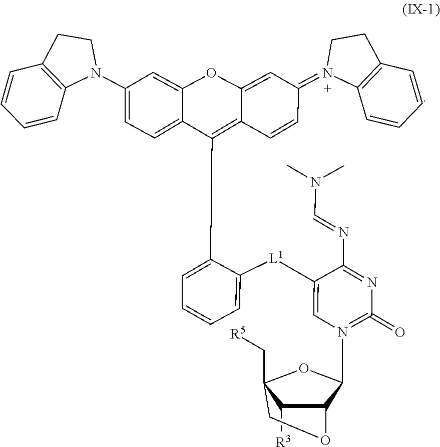

L¹, R³, and R⁵ are as described herein, including in embodiments.

In embodiments, the compound has the formula:

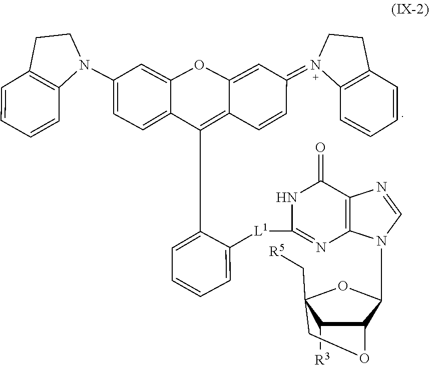

L¹, R³, and R⁵ are as described herein, including in embodiments.

In embodiments, the compound has the formula:

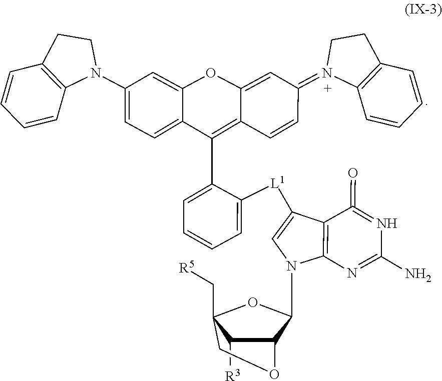

L¹, R³, and R⁵ are as described herein, including in embodiments.

In embodiments, the compound has the formula:

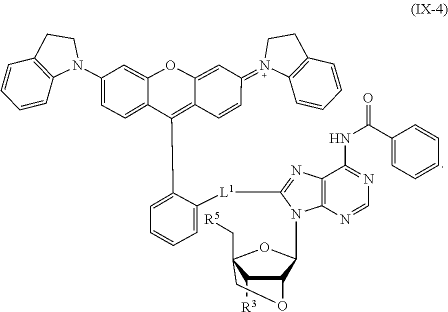

L¹, R³, and R⁵ are as described herein, including in embodiments.

In embodiments, the compound has the formula:

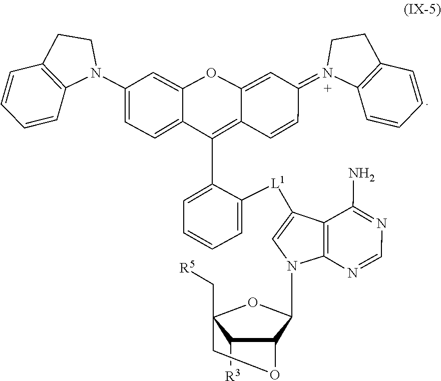

L¹, R³, and R⁵ are as described herein, including in embodiments.

In embodiments, L¹ is a divalent linker including 4 to 30 atoms.

In embodiments, L¹ is L¹⁰¹-L¹⁰²-L¹⁰³-L¹⁰⁴-L¹⁵⁰.

L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L¹⁰¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond. In embodiments, L¹⁰¹ is —NH—. In embodiments, L¹⁰¹ is —O—. In embodiments, L¹⁰¹ is —S—. In embodiments, L¹⁰¹ is —S(O)—. In embodiments, L¹⁰¹ is —S(O)₂—. In embodiments, L¹⁰¹ is —C(O)—. In embodiments, L¹⁰¹ is —C(O)NH—. In embodiments, L¹⁰¹ is —NHC(O)—. In embodiments, L¹⁰¹ is —NHC(O)NH—. In embodiments, L¹⁰¹ is —C(O)O—. In embodiments, L¹⁰¹ is —OC(O)—. In embodiments, L¹⁰¹ is substituted or unsubstituted C₁-C₄ alkylene. In embodiments, L¹⁰¹ is substituted or unsubstituted 2 to 6 membered heteroalkylene.

In embodiments, a substituted L¹⁰² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond. In embodiments, L¹⁰² is —NH—. In embodiments, L¹⁰² is —O—. In embodiments, L¹⁰² is —S—. In embodiments, L¹⁰² is —S(O)—. In embodiments, L¹⁰² is —S(O)₂—. In embodiments, L¹⁰² is —C(O)—. In embodiments, L¹⁰² is —C(O)NH—. In embodiments, L¹⁰² is —NHC(O)—. In embodiments, L¹⁰² is —NHC(O)NH—. In embodiments, L¹⁰² is —C(O)O—. In embodiments, L¹⁰² is —OC(O)—. In embodiments, L¹⁰² is substituted or unsubstituted C₁-C₄ alkylene. In embodiments, L¹⁰² is substituted or unsubstituted 2 to 6 membered heteroalkylene. In embodiments, L¹⁰² is an unsubstituted 3 to 8 membered heterocycloalkyl. In embodiments, L¹⁰² is an unsubstituted piperidinyl. In embodiments, L¹⁰² is

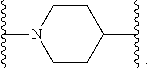 [SMILES: *C1CCN(*)CC1]

In embodiments, a substituted L¹⁰³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond. In embodiments, L¹⁰³ is —NH—. In embodiments, L¹⁰³ is —O—. In embodiments, L¹⁰³ is —S—. In embodiments, L¹⁰³ is —S(O)—. In embodiments, L¹⁰ is —S(O)₂—. In embodiments, L¹⁰³ is —C(O)—. In embodiments, L¹⁰³ is —C(O)NH—. In embodiments, L¹⁰³ is —NHC(O)—. In embodiments, L¹⁰³ is —NHC(O)NH—. In embodiments, L¹⁰³ is —C(O)O—. In embodiments, L¹⁰³ is —OC(O)—. In embodiments, L¹⁰³ is substituted or unsubstituted C₁-C₄ alkylene. In embodiments, L¹⁰³ is substituted or unsubstituted 2 to 6 membered heteroalkylene.

In embodiments, a substituted L¹⁰⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is a bond. In embodiments, L¹⁰⁴ is —NH—. In embodiments, L¹⁰⁴ is —O—. In embodiments, L¹⁰⁴ is —S—. In embodiments, L¹⁰⁴ is —S(O)—. In embodiments, L¹⁰⁴ is —S(O)₂—. In embodiments, L¹⁰⁴ is —C(O)—. In embodiments, L¹⁰⁴ is —C(O)NH—. In embodiments, L¹⁰⁴ is —NHC(O)—. In embodiments, L¹⁰⁴ is —NHC(O)NH—. In embodiments, L¹⁰⁴ is —C(O)O—. In embodiments, L¹⁰⁴ is —OC(O)—. In embodiments, L¹⁰⁴ is an unsubstituted C₁-C₁₀ alkylene. In embodiments, L¹⁰⁴ is an unsubstituted methylene. In embodiments, L¹⁰⁴ is an unsubstituted ethylene. In embodiments, L¹⁰⁴ is an unsubstituted propylene. In embodiments, L¹⁰⁴ is an unsubstituted n-propylene. In embodiments, L¹⁰⁴ is an unsubstituted butylene. In embodiments, L¹⁰⁴ is an unsubstituted n-butylene. In embodiments, L¹⁰⁴ is an unsubstituted pentylene. In embodiments, L¹⁰⁴ is an unsubstituted n-pentylene. In embodiments, L¹⁰⁴ is an unsubstituted hexylene. In embodiments, L¹⁰⁴ is an unsubstituted n-hexylene. In embodiments, L¹⁰⁴ is an unsubstituted heptylene. In embodiments, L¹⁰⁴ is an unsubstituted n-heptylene. In embodiments, L¹⁰⁴ is an unsubstituted octylene. In embodiments, L¹⁰⁴ is an unsubstituted n-octylene. In embodiments, L¹⁰⁴ is an unsubstituted C₂-C₆ alkynylene. In embodiments, L¹⁰⁴ is an unsubstituted ethynylene. In embodiments, L¹⁰⁴ is an unsubstituted propynylene. In embodiments, L¹⁰⁴ is an unsubstituted butynylene. In embodiments, L¹⁰⁴ is an unsubstituted pentynylene. In embodiments, L¹⁰⁴ is an unsubstituted hexynylene. In embodiments, L¹⁰⁴ is

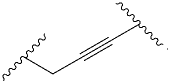 [SMILES: *C#CC*]

In embodiments, L¹⁰⁴ is a substituted or unsubstituted 2 to 6 membered heteroalkylene. In embodiments, L¹⁰⁴ is an unsubstituted 2 to 6 membered heteroalkylene. In embodiments, L¹⁰⁴ is

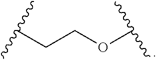 [SMILES: *CCO*]

In embodiments, L¹⁰⁴ is

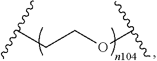

wherein n104 is an integer from 1 to 10. In embodiments, n104 is 1. In embodiments, n104 is 2. In embodiments, n104 is 3. In embodiments, n104 is 4. In embodiments, n104 is 5. In embodiments, n104 is 6. In embodiments, n104 is 7. In embodiments, n104 is 8. In embodiments, n104 is 9. In embodiments, n104 is 10. In embodiments, L¹⁰⁴ is substituted or unsubstituted phenylene. In embodiments, L¹⁰⁴ is unsubstituted phenylene. In embodiments, L¹⁰⁴ is

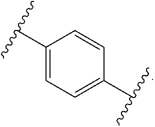 [SMILES: *c1ccc(*)cc1]

In embodiments, a substituted L¹⁰⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is a bond. In embodiments, L¹⁰⁵ is —NH—. In embodiments, L¹⁰⁵ is —O—. In embodiments, L¹⁰⁵ is —S—. In embodiments, L¹⁰⁵ is —S(O)—. In embodiments, L¹⁰⁵ is —S(O)₂—. In embodiments, L¹⁰⁵ is —C(O)—. In embodiments, L¹⁰⁵ is —C(O)NH—. In embodiments, L¹⁰⁵ is —NHC(O)—. In embodiments, L¹⁰⁵ is —NHC(O)NH—. In embodiments, L¹⁰⁵ is —C(O)O—. In embodiments, L¹⁰⁵ is —OC(O)—. In embodiments, L¹⁰⁵ is an unsubstituted C₁-C₁₀ alkylene. In embodiments, L¹⁰⁵ is substituted or unsubstituted C₁-C₄ alkylene. In embodiments, L¹¹⁵ is an unsubstituted methylene. In embodiments, L¹⁰⁵ is an unsubstituted ethylene. In embodiments, L¹⁰⁵ is an unsubstituted propylene. In embodiments, L¹⁰⁵ is an unsubstituted n-propylene. In embodiments, L¹⁰⁵ is an unsubstituted butylene. In embodiments, L¹⁰⁵ is an unsubstituted n-butylene. In embodiments, L¹⁰⁵ is an unsubstituted pentylene. In embodiments, L¹⁰⁵ is an unsubstituted n-pentylene. In embodiments, L¹⁰⁵ is an unsubstituted hexylene. In embodiments, L¹⁰⁵ is an unsubstituted n-hexylene. In embodiments, L¹⁰⁵ is an unsubstituted heptylene. In embodiments, L¹⁰⁵ is an unsubstituted n-heptylene. In embodiments, L¹⁰⁵ is an unsubstituted octylene. In embodiments, L¹⁰⁵ is an unsubstituted n-octylene. In embodiments, L¹⁰⁵ is an unsubstituted C₂-C₆ alkynylene. In embodiments, L¹⁰⁵ is an unsubstituted ethynylene. In embodiments, L¹⁰⁵ is an unsubstituted propynylene. In embodiments, L¹⁰⁵ is an unsubstituted butynylene. In embodiments, L¹⁰⁵ is an unsubstituted pentynylene. In embodiments, L¹⁰⁵ is an unsubstituted hexynylene. In embodiments, L¹⁰⁵ is

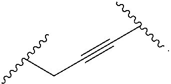 [SMILES: *C#CC*]

In embodiments, L¹⁰⁵ is a substituted 2 to 8 membered heteroalkylene. In embodiments, L¹⁰⁵ is an oxo-substituted 2 to 8 membered heteroalkylene. In embodiments, L¹⁰⁵ is an oxo-substituted 2 to 8 membered heteroalkenylene. In embodiments, L¹⁰⁵ is

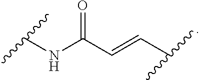 [SMILES: */C=C/C(=O)N*]

In embodiments, L¹⁰⁵ is

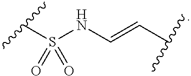 [SMILES: */C=C/NS(*)(=O)=O]

In embodiments, L¹⁰⁵ is

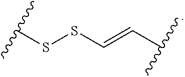 [SMILES: */C=C/SS*]

In embodiments, L¹⁰⁵ is a substituted or unsubstituted 2 to 8 membered heteroalkynylene. In embodiments, L¹⁰⁵ is

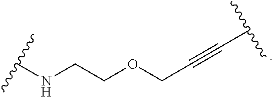 [SMILES: *C#CCOCCN*]

In embodiments, L¹⁰⁵ is

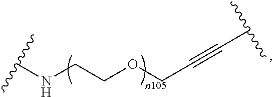

wherein n105 is an integer from 1 to 10. In embodiments, n105 is 1. In embodiments, n105 is 2. In embodiments, n105 is 3. In embodiments, n105 is 4. In embodiments, n105 is 5. In embodiments, n105 is 6. In embodiments, n105 is 7. In embodiments, n105 is 8. In embodiments, n105 is 9. In embodiments, n105 is 10. In embodiments, L¹⁰⁵ is an unsubstituted 5 to 10 membered heteroarylene. In embodiments, L¹⁰⁵ is an unsubstituted triazolylene. In embodiments, L¹⁰⁵ is

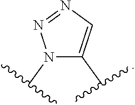 [SMILES: *c1cnnn1*]

In embodiments, L¹ is

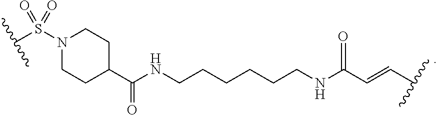 [SMILES: */C=C/C(=O)NCCCCCCNC(=O)C1CCN(S(*)(=O)=O)CC1]

In embodiments, L¹ is

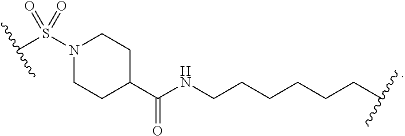 [SMILES: *CCCCCCNC(=O)C1CCN(S(*)(=O)=O)CC1]

In embodiments, L¹ is

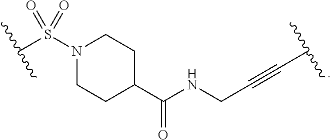 [SMILES: *C#CCNC(=O)C1CCN(S(*)(=O)=O)CC1]

In embodiments, L¹ is

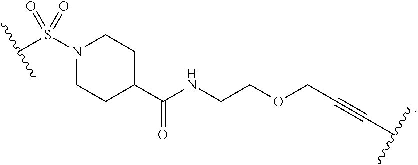 [SMILES: *C#CCOCCNC(=O)C1CCN(S(*)(=O)=O)CC1]

In embodiments, L¹ is

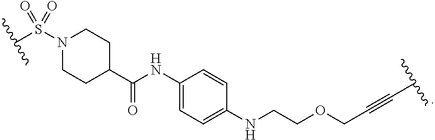 [SMILES: *C#CCOCCNc1ccc(NC(=O)C2CCN(S(*)(=O)=O)CC2)cc1]

In embodiments, L¹ is

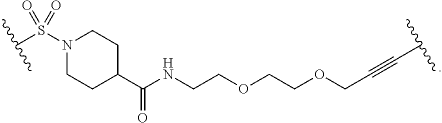 [SMILES: *C#CCOCCOCCNC(=O)C1CCN(S(*)(=O)=O)CC1]

In embodiments, L¹ is

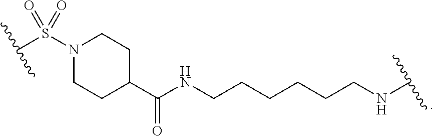 [SMILES: *NCCCCCCNC(=O)C1CCN(S(*)(=O)=O)CC1]

In embodiments, L¹ is

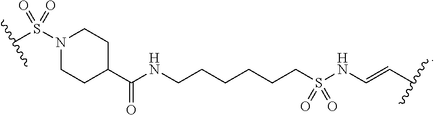 [SMILES: */C=C/NS(=O)(=O)CCCCCCNC(=O)C1CCN(S(*)(=O)=O)CC1]

In embodiments, L¹ is

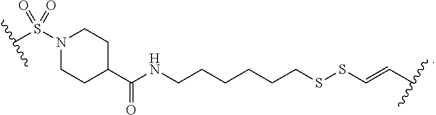 [SMILES: */C=C/SSCCCCCCNC(=O)C1CCN(S(*)(=O)=O)CC1]

In embodiments, L¹ is

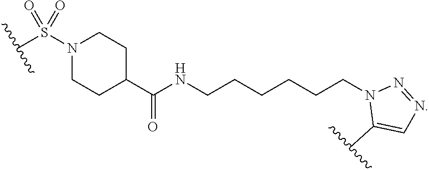 [SMILES: *c1cnnn1CCCCCCNC(=O)C1CCN(S(*)(=O)=O)CC1]

In embodiments, a substituted R¹ (e.g., substituted alkyl and/or substituted heteroalkyl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, Ri is unsubstituted isobutyl. In embodiments, R¹ is unsubstituted tert-butyl.

In embodiments, R² is hydrogen or —OH. In embodiments, R² is hydrogen. In embodiments, R² is —OR^{2A}. In embodiments, R² is —OH.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, R^{2A} is hydrogen. In embodiments, R^{2A} is unsubstituted C₁-C₄ alkyl. In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R³ is —OR^{3A}. In embodiments, R³ is —OH. In embodiments, R³ is —O—P(NR^{3B}R^{3C})—OR^{3A}. In embodiments, R³ is

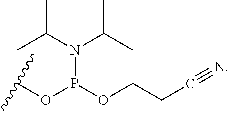 [SMILES: *OP(OCCC#N)N(C(C)C)C(C)C]

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl. In embodiments, R^{3A} is substituted C₁-C₄ alkyl. In embodiments, R^{3A} is cyano-substituted C₁-C₄ alkyl. In embodiments, R^{A} is

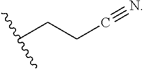 [SMILES: *CCC#N]

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, R^{3C} is hydrogen. In embodiments, R^{3C} is unsubstituted C₁-C₄ alkyl. In embodiments, R^{C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R⁴ is hydrogen. In embodiments, R⁴ is unsubstituted methyl.

In embodiments, a substituted ring formed when R² and R⁴ substituents are joined (e.g., substituted heterocycloalkyl) is substituted with at least one substituent group, size-limited substituent group, or lower substituent group; wherein if the substituted ring formed when R² and R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R⁴ substituents are joined is substituted, it is substituted with at least one substituent group. In embodiments, when the substituted ring formed when R² and R⁴ substituents are joined is substituted, it is substituted with at least one size-limited substituent group. In embodiments, when the substituted ring formed when R² and R⁴ substituents are joined is substituted, it is substituted with at least one lower substituent group.

In embodiments, R² and R⁴ substituents are joined to form a substituted or unsubstituted 3 to 8 membered heterocycloalkyl. In embodiments, R² and R⁴ substituents are joined to form a substituted or unsubstituted tetrahydrofuranyl.

In embodiments, R⁵ is —OH.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, R^{5A} is hydrogen or substituted C₁-C₆ alkyl. In embodiments, R^{5A} is hydrogen. In embodiments, R^{5A} is unsubstituted C₁-C₄ alkyl. In embodiments, R^{5A} is unsubstituted methyl. In embodiments, R^{5A} is unsubstituted ethyl. In embodiments, R^{5A} is unsubstituted propyl. In embodiments, R^{5A} is unsubstituted n-propyl. In embodiments, R^{5A} is unsubstituted isopropyl. In embodiments, R^{5A} is unsubstituted butyl. In embodiments, R^{5A} is unsubstituted n-butyl. In embodiments, R^{5A} is unsubstituted isobutyl. In embodiments, R^{5A} is unsubstituted tert-butyl. In embodiments, R^{5A} is substituted C₁-C₆ alkyl. In embodiments, R^{5A} is dimethoxytrityl. In embodiments, R^{5A} is

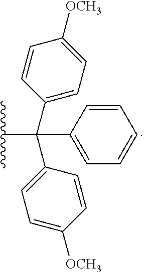 [SMILES: *C(c1ccccc1)(c1ccc(OC)cc1)c1ccc(OC)cc1]

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group. In embodiments, R⁶ is hydrogen. In embodiments, R⁶ is halogen. In embodiments, R⁶ is —F. In embodiments, R⁶ is —Cl. In embodiments, R⁶ is —Br. In embodiments, R⁶ is —I. In embodiments, R⁶ is —CCl₃. In embodiments, R⁶ is —CBr₃. In embodiments, R⁶ is —CF₃. In embodiments, R⁶ is —CI₃. In embodiments, R⁶ is —CH₂Cl. In embodiments, R⁶ is —CH₂Br. In embodiments, R⁶ is —CH₂F. In embodiments, R⁶ is —CH₂I. In embodiments, R⁶ is —CHCl₂. In embodiments, R⁶ is —CHBr₂. In embodiments, R⁶ is —CHF₂. In embodiments, R⁶ is —CHI₂. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃R^{A}. In embodiments, R⁶ is —SO₃H. In embodiments, R⁶ is —SO₂NH₂. In embodiments, R⁶ is □NHNH₂. In embodiments, R⁶ is □ONH₂. In embodiments, R⁶ is □NHC(O)NH₂. In embodiments, R⁶ is —NHSO₂H. In embodiments, R⁶ is —NHC(O)H. In embodiments, R⁶ is —NHC(O)OH. In embodiments, R⁶ is —NHOH. In embodiments, R⁶ is —OCCl₃. In embodiments, R⁶ is —OCBr₃. In embodiments, R⁶ is —OCF₃. In embodiments, R⁶ is —OCI₃. In embodiments, R⁶ is —OCH₂Cl. In embodiments, R⁶ is —OCH₂Br. In embodiments, R⁶ is —OCH₂F. In embodiments, R⁶ is —OCH₂I. In embodiments, R⁶ is —OCHCl₂. In embodiments, R⁶ is —OCHBr₂. In embodiments, R⁶ is —OCHF₂. In embodiments, R⁶ is —OCHI₂. In embodiments, R⁶ is —SF₅. In embodiments, R⁶ is —N₃. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is unsubstituted 2 to 6 membered heteroalkyl. In embodiments, R⁶ is unsubstituted methoxy. In embodiments, R⁶ is unsubstituted ethoxy. In embodiments, R⁶ is unsubstituted propoxy. In embodiments, R⁶ is unsubstituted n-propoxy. In embodiments, R⁶ is unsubstituted isopropoxy. In embodiments, R⁶ is unsubstituted butoxy.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group. In embodiments, R⁷ is hydrogen. In embodiments, R⁷ is halogen. In embodiments, R⁷ is —F. In embodiments, R⁷ is —Cl. In embodiments, R⁷ is —Br. In embodiments, R⁷ is —I. In embodiments, R⁷ is —CCl₃. In embodiments, R⁷ is —CBr₃. In embodiments, R⁷ is —CF₃. In embodiments, R⁷ is —CI₃. In embodiments, R⁷ is —CH₂Cl. In embodiments, R⁷ is —CH₂Br. In embodiments, R⁷ is —CH₂F. In embodiments, R⁷ is —CH₂I. In embodiments, R⁷ is —CHCl₂. In embodiments, R⁷ is —CHBr₂. In embodiments, R⁷ is —CHF₂. In embodiments, R⁷ is —CH₁₂. In embodiments, R⁷ is —CN. In embodiments, R⁷ is —OH. In embodiments, R⁷ is —NH₂. In embodiments, R⁷ is —COOH. In embodiments, R⁷ is —CONH₂. In embodiments, R⁷ is —NO₂. In embodiments, R⁷ is —SH. In embodiments, R⁷ is —SO—R^{A}. In embodiments, R⁷ is —SO₃H. In embodiments, R⁷ is —SO₂NH₂. In embodiments, R⁷ is □NHNH₂. In embodiments, R⁷ is □ONH₂. In embodiments, R⁷ is □NHC(O)NH₂. In embodiments, R⁷ is —NHSO₂H. In embodiments, R⁷ is —NHC(O)H. In embodiments, R⁷ is —NHC(O)OH. In embodiments, R⁷ is —NHOH. In embodiments, R⁷ is —OCCl₃. In embodiments, R⁷ is —OCBr₃. In embodiments, R⁷ is —OCF₃. In embodiments, R⁷ is —OCI₃. In embodiments, R⁷ is —OCH₂Cl. In embodiments, R⁷ is —OCH₂Br. In embodiments, R⁷ is —OCH₂F. In embodiments, R⁷ is —OCH₂I. In embodiments, R⁷ is —OCHCl₂. In embodiments, R⁷ is —OCHBr₂. In embodiments, R⁷ is —OCHF₂. In embodiments, R⁷ is —OCHI₂. In embodiments, R⁷ is —SF₅. In embodiments, R⁷ is —N₃. In embodiments, R⁷ is unsubstituted C₁-C₄ alkyl. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is unsubstituted 2 to 6 membered heteroalkyl. In embodiments, R⁷ is unsubstituted methoxy. In embodiments, R⁷ is unsubstituted ethoxy. In embodiments, R⁷ is unsubstituted propoxy. In embodiments, R⁷ is unsubstituted n-propoxy. In embodiments, R⁷ is unsubstituted isopropoxy. In embodiments, R⁷ is unsubstituted butoxy.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen. In embodiments, R⁸ is halogen. In embodiments, R⁸ is —F. In embodiments, R⁸ is —Cl. In embodiments, R⁸ is —Br. In embodiments, R⁸ is —I. In embodiments, R⁸ is —CCl₃. In embodiments, R⁸ is —CBr₃. In embodiments, R⁸ is —CF₃. In embodiments, R⁸ is —CI₃. In embodiments, R⁸ is —CH₂Cl. In embodiments, R⁸ is —CH₂Br. In embodiments, R⁸ is —CH₂F. In embodiments, R⁵ is —CH₂I. In embodiments, R⁸ is —CHCl₂. In embodiments, R⁸ is —CHBr₂. In embodiments, R⁸ is —CHF₂. In embodiments, R⁸ is —CHI₂. In embodiments, R⁸ is —CN. In embodiments, R⁸ is —OH. In embodiments, R⁸ is —NH₂. In embodiments, R⁸ is —COOH. In embodiments, R⁸ is —CONH₂. In embodiments, R⁸ is —NO₂. In embodiments, R⁸ is —SH. In embodiments, R⁸ is —SO₃R^{A}. In embodiments, R⁸ is —SO₃H. In embodiments, R⁸ is —SO₂NH₂. In embodiments, R⁸ is □NHNH₂. In embodiments, R⁸ is □ONH₂. In embodiments, R⁸ is □NHC(O)NH₂. In embodiments, R⁸ is —NHSO₂H. In embodiments, RY is —NHC(O)H. In embodiments, R⁸ is —NHC(O)OH. In embodiments, R⁸ is —NHOH. In embodiments, R⁸ is —OCCl₃. In embodiments, R⁸ is —OCBr₃. In embodiments, R⁸ is —OCF₃. In embodiments, R⁸ is —OCI₃. In embodiments, R⁸ is —OCH₂Cl. In embodiments, R⁸ is —OCH₂Br. In embodiments, R⁸ is —OCH₂F. In embodiments, R⁸ is —OCH₂I. In embodiments, R⁸ is —OCHCl₂. In embodiments, R⁸ is —OCHBr₂. In embodiments, R⁸ is —OCHF₂. In embodiments, R⁸ is —OCHI₂. In embodiments, R⁸ is —SF₅. In embodiments, R⁸ is —N₃. In embodiments, R⁸ is unsubstituted C₁-C₄ alkyl. In embodiments, R⁸ is unsubstituted methyl. In embodiments, R⁸ is unsubstituted ethyl. In embodiments, R⁸ is unsubstituted propyl. In embodiments, R⁸ is unsubstituted n-propyl. In embodiments, R⁸ is unsubstituted isopropyl. In embodiments, R⁸ is unsubstituted butyl. In embodiments, R⁸ is unsubstituted n-butyl. In embodiments, R⁸ is unsubstituted isobutyl. In embodiments, R⁸ is unsubstituted tert-butyl. In embodiments, R⁸ is unsubstituted 2 to 6 membered heteroalkyl. In embodiments, R⁸ is unsubstituted methoxy. In embodiments, R⁸ is unsubstituted ethoxy. In embodiments, R⁸ is unsubstituted propoxy. In embodiments, R⁸ is unsubstituted n-propoxy. In embodiments, R⁸ is unsubstituted isopropoxy. In embodiments, R⁸ is unsubstituted butoxy.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is hydrogen. In embodiments, R⁹ is halogen. In embodiments, R⁹ is —F. In embodiments, R⁹ is —Cl. In embodiments, R⁹ is —Br. In embodiments, R⁹ is —I. In embodiments, R⁹ is —CCl₃. In embodiments, R⁹ is —CBr₃. In embodiments, R⁹ is —CF₃. In embodiments, R⁹ is —CI₃. In embodiments, R⁹ is —CH₂Cl. In embodiments, R⁹ is —CH₂Br. In embodiments, R⁹ is —CH₂F. In embodiments, R⁹ is —CH₂I. In embodiments, R⁹ is —CHCl₂. In embodiments, R⁹ is —CHBr₂. In embodiments, R⁹ is —CHF₂. In embodiments, R⁹ is —CHI₂. In embodiments, R⁹ is —CN. In embodiments, R⁹ is —OH. In embodiments, R⁹ is —NH₂. In embodiments, R⁹ is —COOH. In embodiments, R⁹ is —CONH₂. In embodiments, R⁹ is —NO₂. In embodiments, R⁹ is —SH. In embodiments, R⁹ is —SO₃R^{A}. In embodiments, R⁹ is —SO₃H. In embodiments, R⁹ is —SO₂NH₂. In embodiments, R⁹ is □NHNH₂. In embodiments, R⁹ is □ONH₂. In embodiments, R⁹ is □NHC(O)NH₂. In embodiments. R⁹ is —NHSO₂H. In embodiments, R⁹ is —NHC(O)H. In embodiments, R⁹ is —NHC(O)OH. In embodiments, R⁹ is —NHOH. In embodiments, R⁹ is —OCCl₃. In embodiments, R⁹ is —OCBr₃. In embodiments, R⁹ is —OCF₃. In embodiments, R⁹ is —OCI₃. In embodiments, R⁹ is —OCH₂Cl. In embodiments, R⁹ is —OCH₂Br. In embodiments, R⁹ is —OCH₂F. In embodiments, R⁹ is —OCH₂I. In embodiments, R⁹ is —OCHCl₂. In embodiments, R⁹ is —OCHBr₂. In embodiments, R⁹ is —OCHF₂. In embodiments, R⁹ is —OCHI₂. In embodiments, R⁹ is —SF₅. In embodiments, R⁹ is —N₃. In embodiments, R⁹ is unsubstituted C₁-C₄ alkyl. In embodiments, R⁹ is unsubstituted methyl. In embodiments, R⁹ is unsubstituted ethyl. In embodiments, R⁹ is unsubstituted propyl. In embodiments, R⁹ is unsubstituted n-propyl. In embodiments, R⁹ is unsubstituted isopropyl. In embodiments, R⁹ is unsubstituted butyl. In embodiments, R⁹ is unsubstituted n-butyl. In embodiments, R⁹ is unsubstituted isobutyl. In embodiments, R⁹ is unsubstituted tert-butyl. In embodiments, R⁹ is unsubstituted 2 to 6 membered heteroalkyl. In embodiments, R⁹ is unsubstituted methoxy. In embodiments, R⁹ is unsubstituted ethoxy. In embodiments, R⁹ is unsubstituted propoxy. In embodiments, R⁹ is unsubstituted n-propoxy. In embodiments, R⁹ is unsubstituted isopropoxy. In embodiments, R⁹ is unsubstituted butoxy.

In embodiments, a substituted R^{A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A} is substituted with a plurality of groups selected from substituent groups, size-limited substituent groups, and lower substituent groups; each substituent group, size-limited substituent group, and/or lower substituent group may optionally be different. In embodiments, when R^{A} is substituted, it is substituted with at least one substituent group. In embodiments, when R^{A} is substituted, it is substituted with at least one size-limited substituent group. In embodiments, when R^{A} is substituted, it is substituted with at least one lower substituent group.

In embodiments, R^{A} is hydrogen. In embodiments, R^{A} is unsubstituted C₁-C₄ alkyl. In embodiments, R^{A} is unsubstituted methyl. In embodiments, R^{A} is unsubstituted ethyl. In embodiments, R^{A} is unsubstituted propyl. In embodiments, R^{A} is unsubstituted n-propyl. In embodiments, R^{A} is unsubstituted isopropyl. In embodiments, R^{A} is unsubstituted butyl. In embodiments, R^{A} is unsubstituted n-butyl. In embodiments, R^{A} is unsubstituted isobutyl. In embodiments, R^{A} is unsubstituted tert-butyl.

In embodiments, a substituted R¹⁰ (e.g., substituted alkyl and/or substituted heteroalkyl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl.

In embodiments, a substituted ring formed when R¹ and R⁶ substituents are joined (e.g., substituted heterocycloalkyl and/or substituted heteroaryl) is substituted with at least one substituent group, size-limited substituent group, or lower substituent group; wherein if the substituted ring formed when R¹ and R⁶ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹ and R⁶ substituents are joined is substituted, it is substituted with at least one substituent group. In embodiments, when the substituted ring formed when R¹ and R⁶ substituents are joined is substituted, it is substituted with at least one size-limited substituent group. In embodiments, when the substituted ring formed when R¹ and R⁶ substituents are joined is substituted, it is substituted with at least one lower substituent group.

In embodiments, R¹ and R⁶ substituents are joined to form a substituted or unsubstituted 3 to 8 membered heterocycloalkyl. In embodiments, R¹ and R⁶ substituents are joined to form a substituted or unsubstituted pyrrolidinyl. In embodiments, R¹ and R⁶ substituents are joined to form an unsubstituted pyrrolidinyl. In embodiments, R¹ and R⁶ substituents are joined to form a substituted or unsubstituted 5 to 6 membered heteroaryl.

In embodiments, a substituted ring formed when R⁸ and R¹⁰ substituents are joined (e.g., substituted heterocycloalkyl and/or substituted heteroaryl) is substituted with at least one substituent group, size-limited substituent group, or lower substituent group; wherein if the substituted ring formed when R⁸ and R¹⁰ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁸ and R¹⁰ substituents are joined is substituted, it is substituted with at least one substituent group. In embodiments, when the substituted ring formed when R⁸ and R¹⁰ substituents are joined is substituted, it is substituted with at least one size-limited substituent group. In embodiments, when the substituted ring formed when R⁸ and R¹⁰ substituents are joined is substituted, it is substituted with at least one lower substituent group.

In embodiments, R⁸ and R¹⁰ substituents are joined to form a substituted or unsubstituted 3 to 8 membered heterocycloalkyl. In embodiments, R⁸ and R¹⁰ substituents are joined to form a substituted or unsubstituted pyrrolidinyl. In embodiments, R⁸ and R¹⁰ substituents are joined to form an unsubstituted pyrrolidinyl. In embodiments, R⁸ and R¹⁰ substituents are joined to form a substituted or unsubstituted 5 to 6 membered heteroaryl.

In embodiments, the compound has the formula:

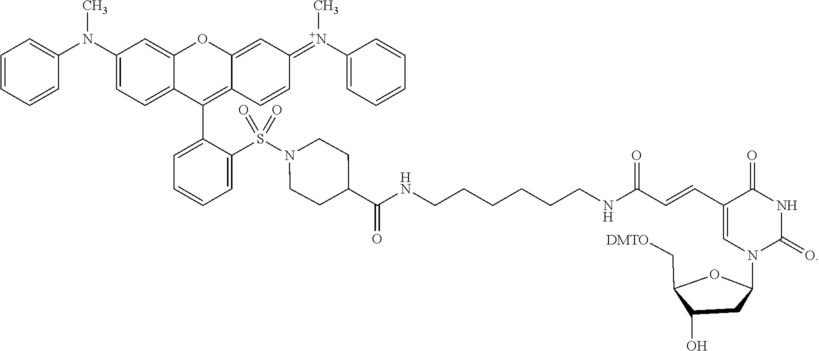

In embodiments, the compound has the formula:

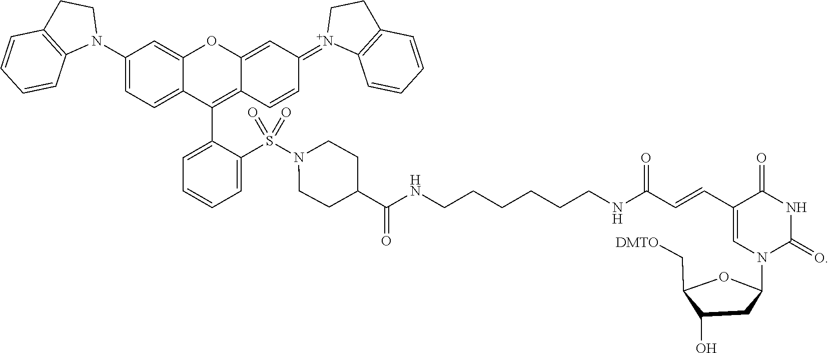

In embodiments, the compound has the formula:

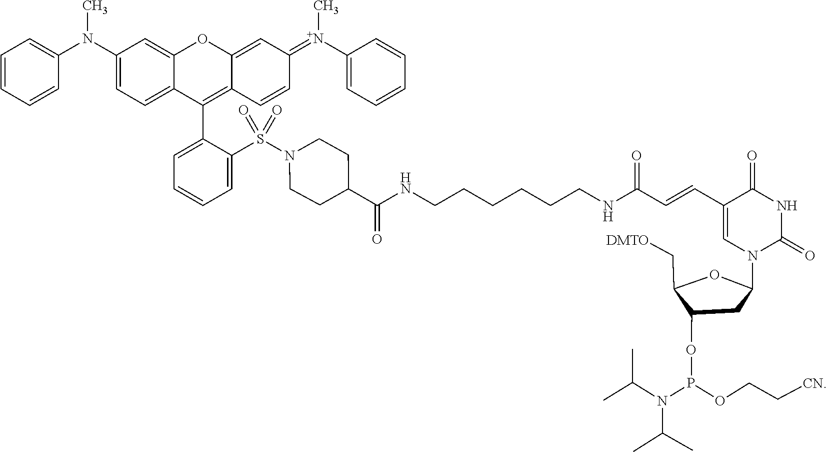

In embodiments, the compound has the formula:

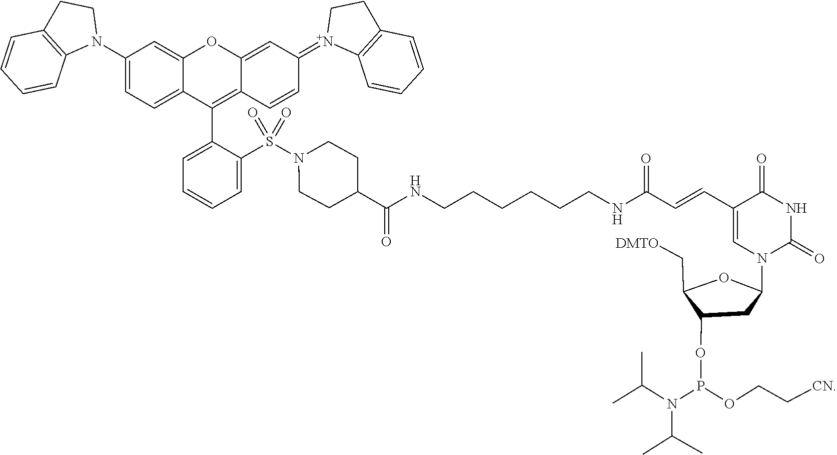

In embodiments, the compound has the formula:

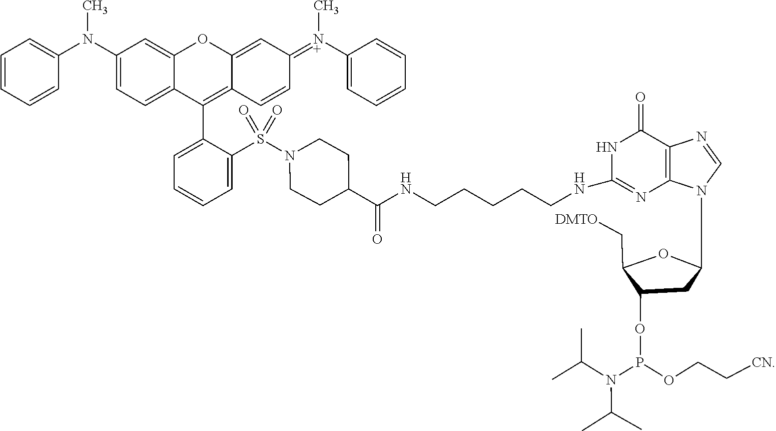

In embodiments, the compound has the formula:

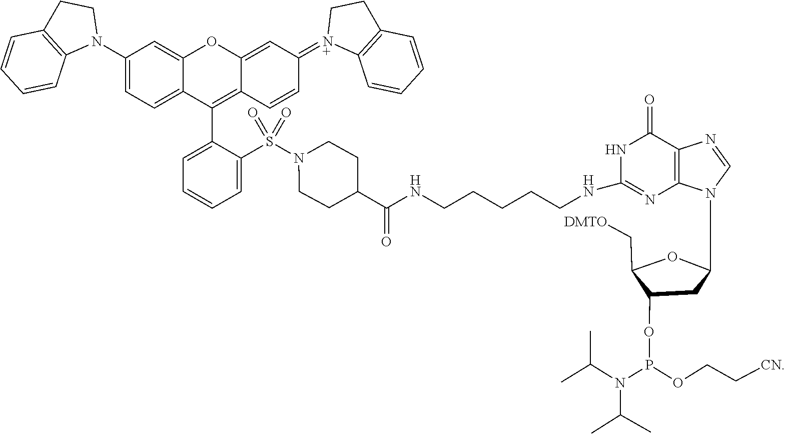

In embodiments, the compound has the formula:

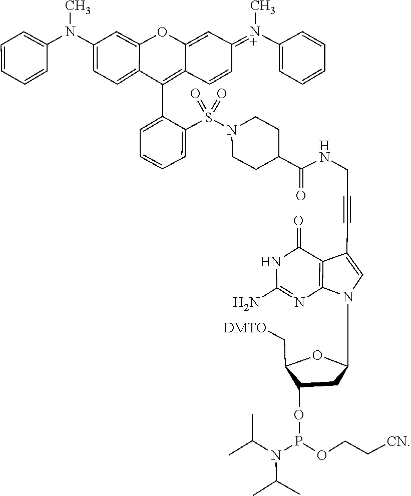

In embodiments, the compound has the formula:

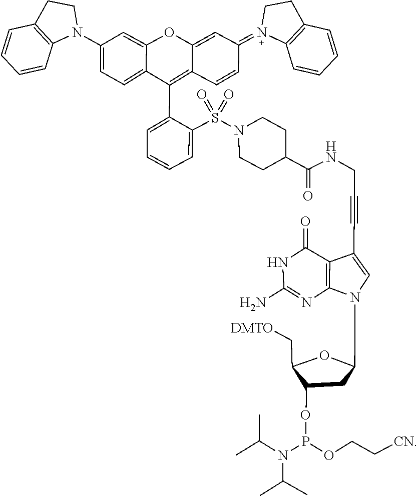

In embodiments, the compound has the formula:

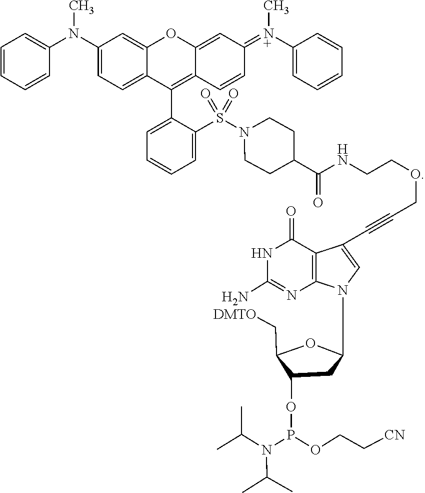

In embodiments, the compound has the formula:

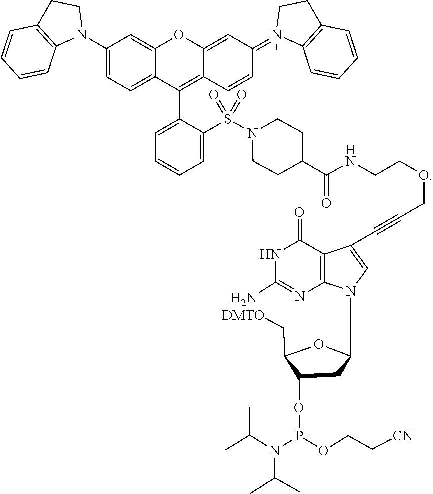

In embodiments, the compound has the formula:

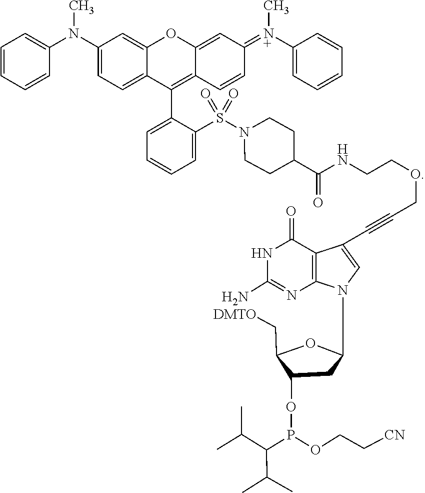

In embodiments, the compound has the formula:

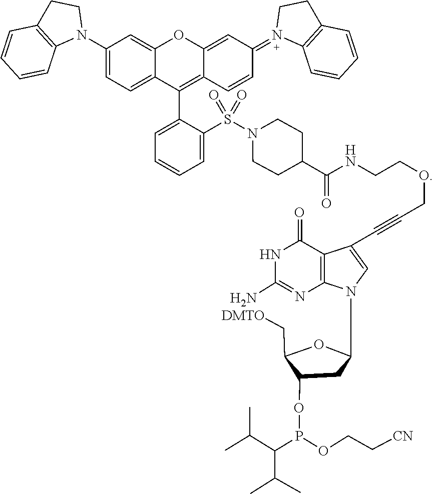

In embodiments, the compound has the formula:

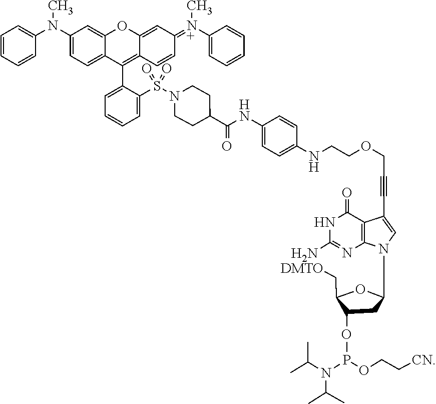

In embodiments, the compound has the formula:

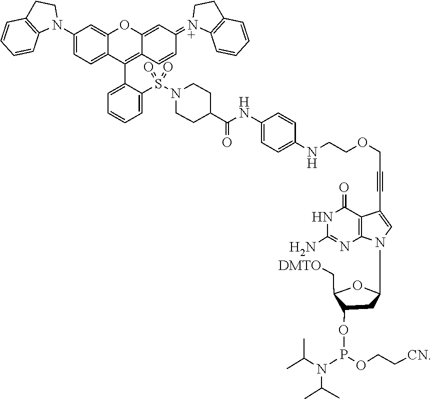

In embodiments, the compound has the formula:

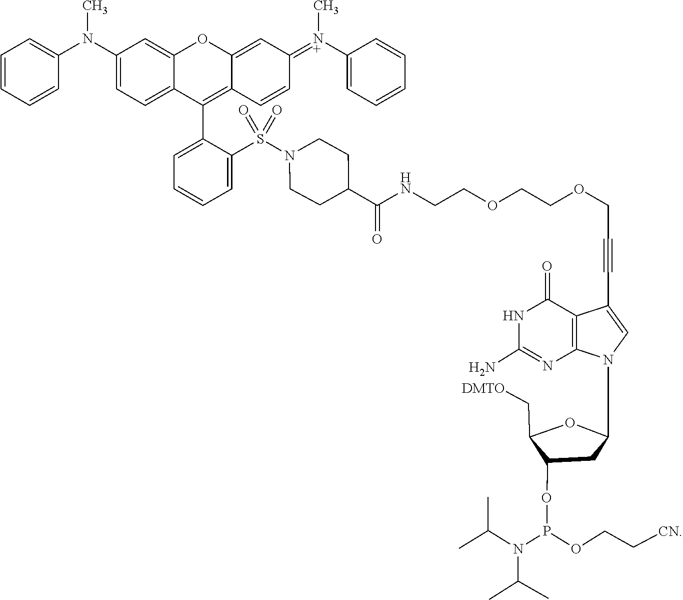

In embodiments, the compound has the formula:

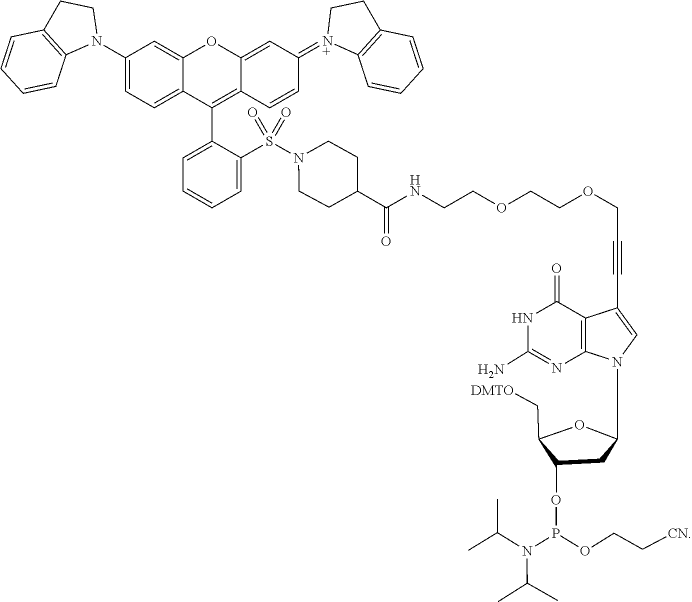

In embodiments, the compound has the formula:

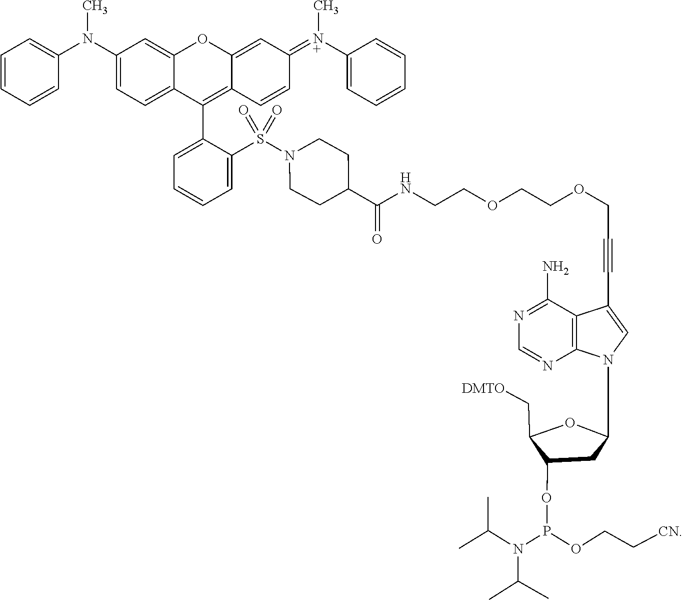

In embodiments, the compound has the formula:

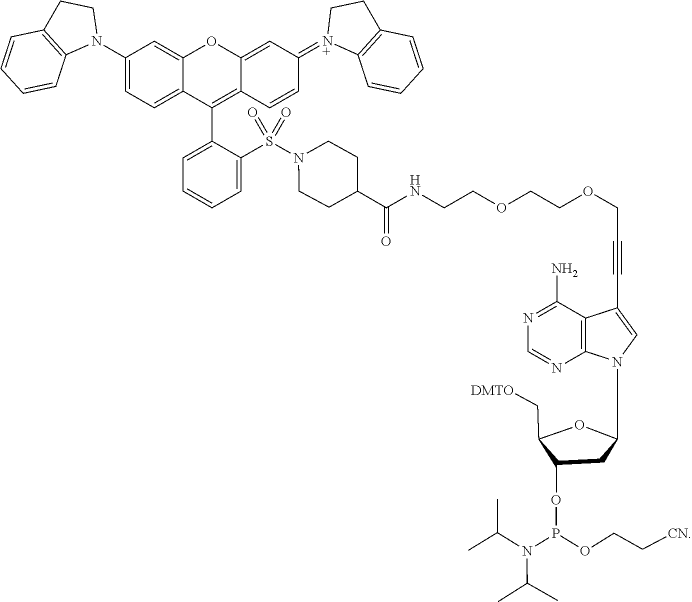

In embodiments, the compound has the formula:

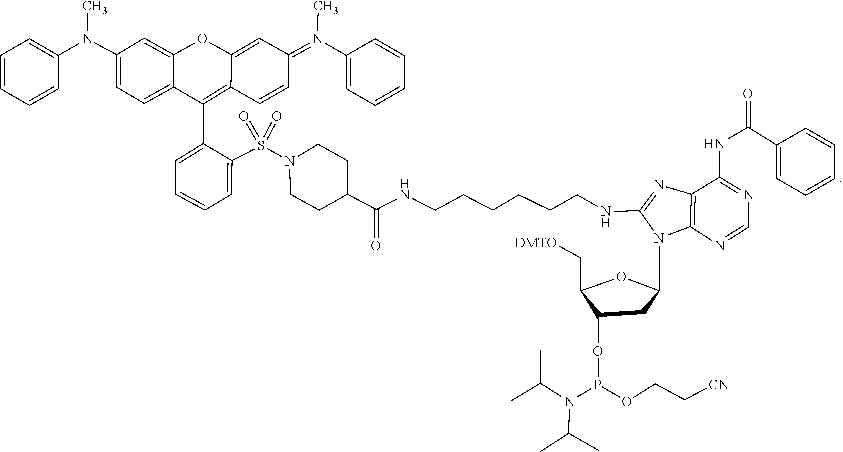

In embodiments, the compound has the formula:

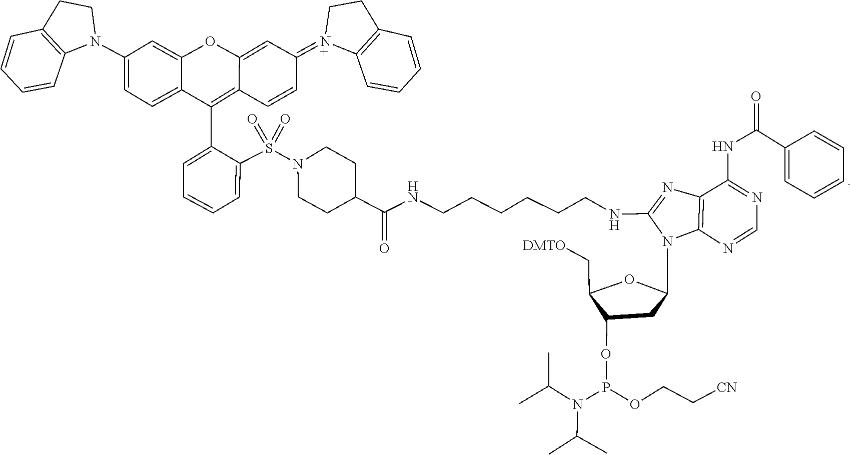

In embodiments, the compound has the formula:

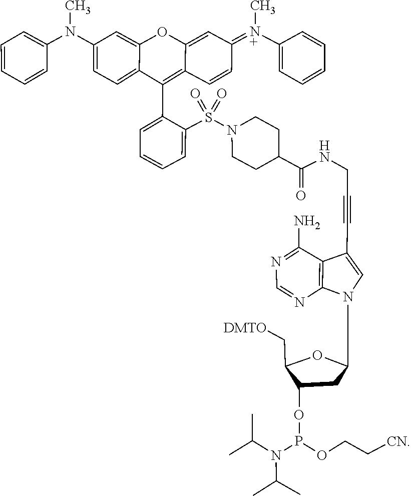

In embodiments, the compound has the formula:

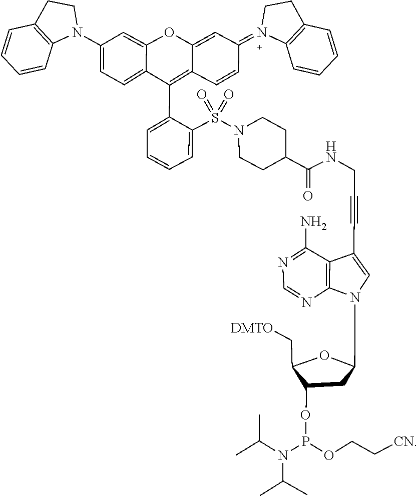

In embodiments, the compound has the formula:

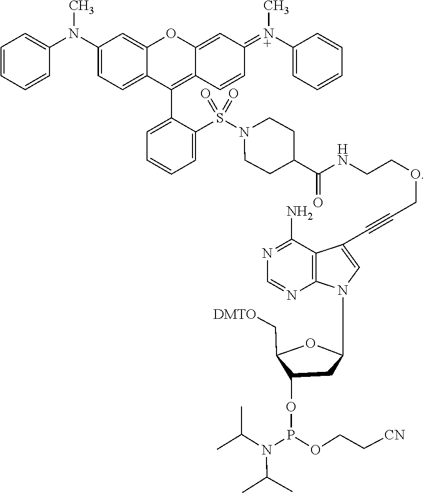

In embodiments, the compound has the formula:

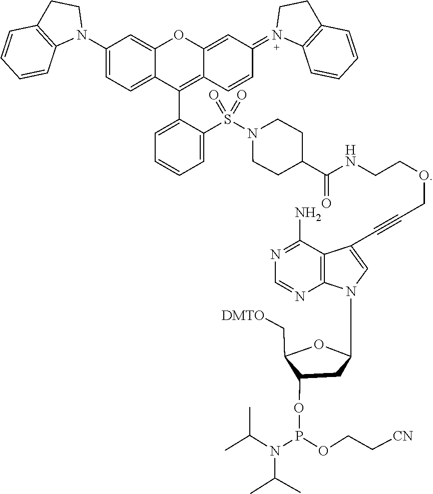

In embodiments, the compound has the formula:

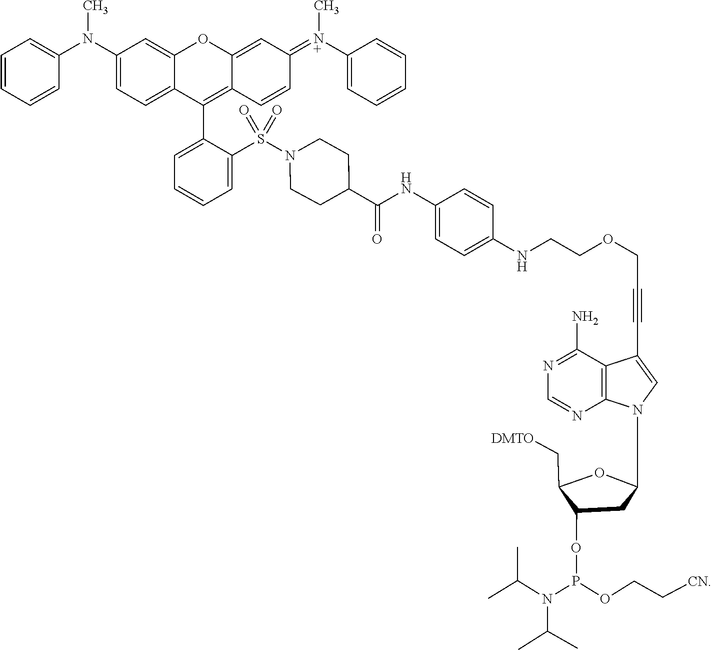

In embodiments, the compound has the formula:

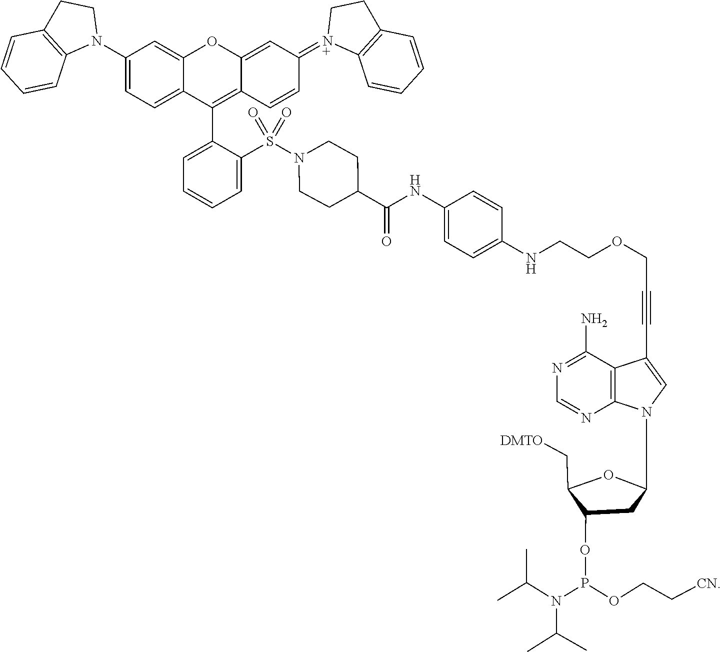

In embodiments, the compound has the formula:

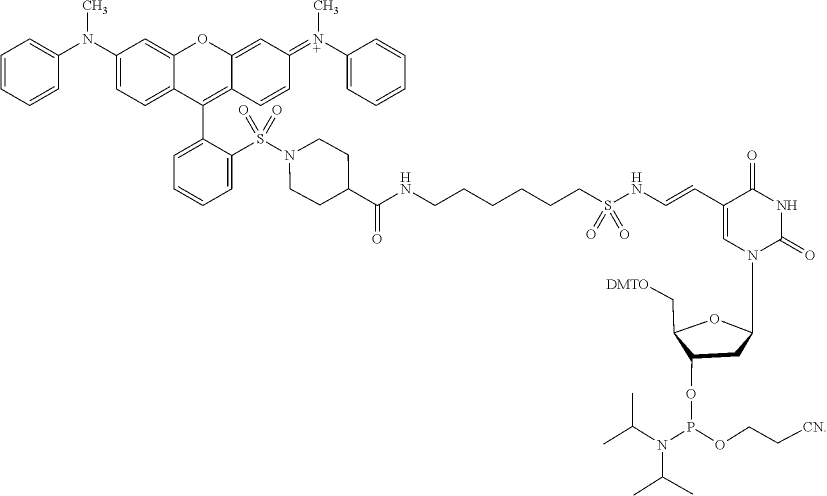

In embodiments, the compound has the formula:

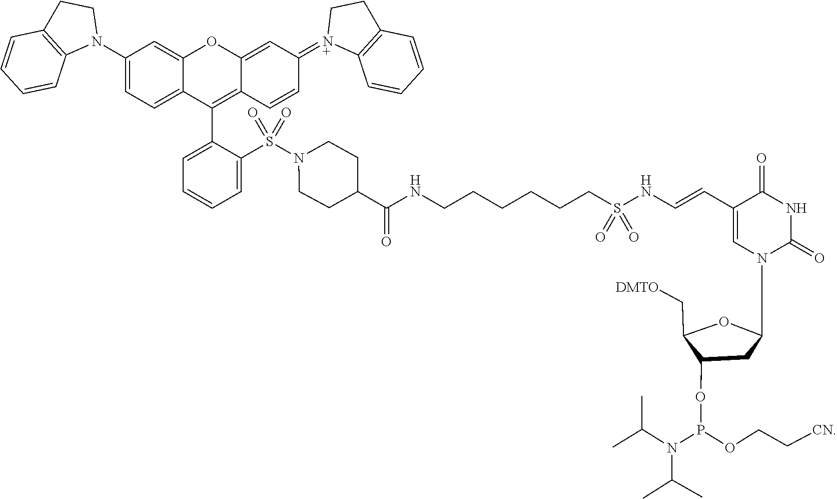

In embodiments, the compound has the formula:

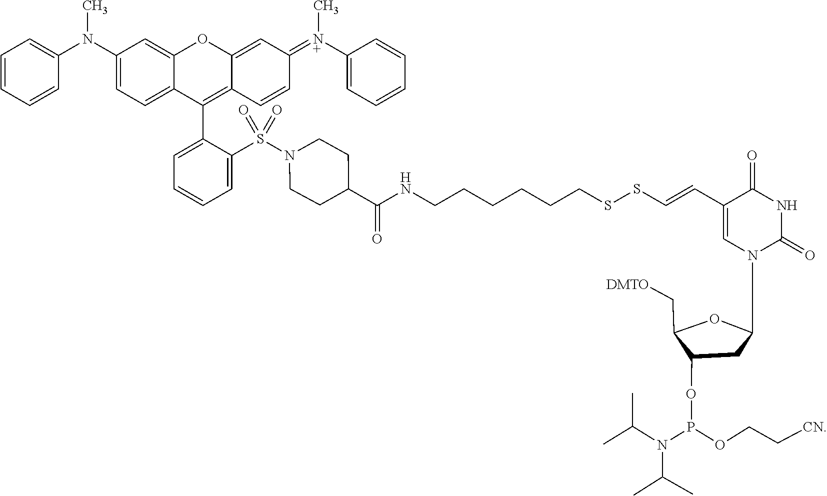

In embodiments, the compound has the formula:

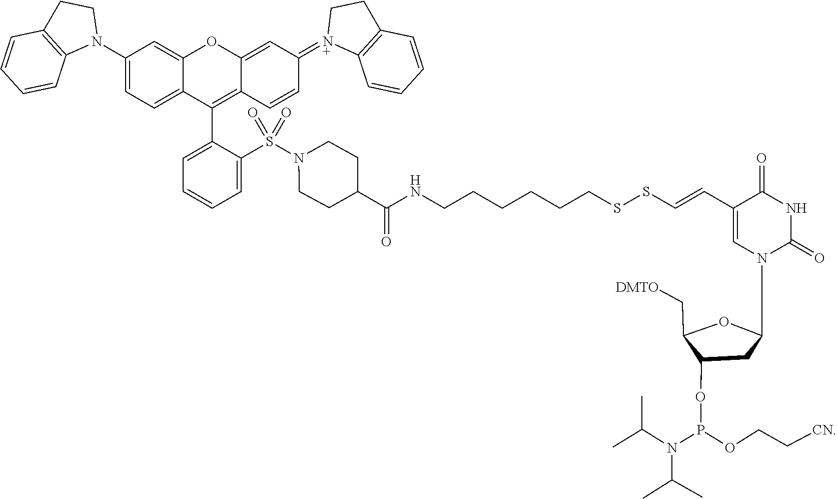

In embodiments, the compound has the formula:

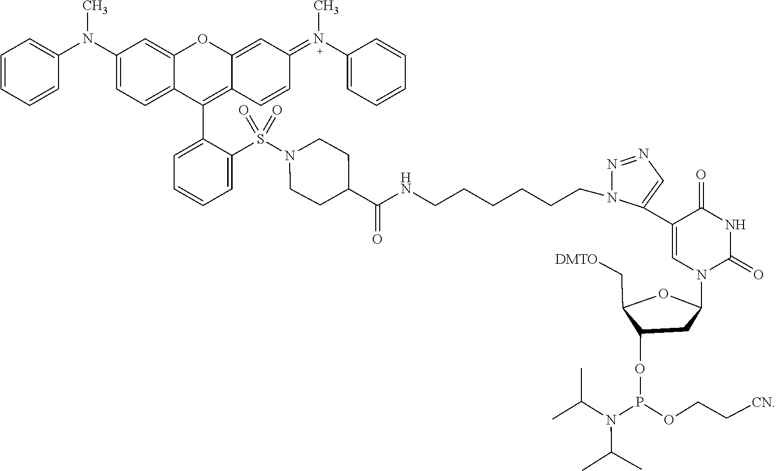

In embodiments, the compound has the formula:

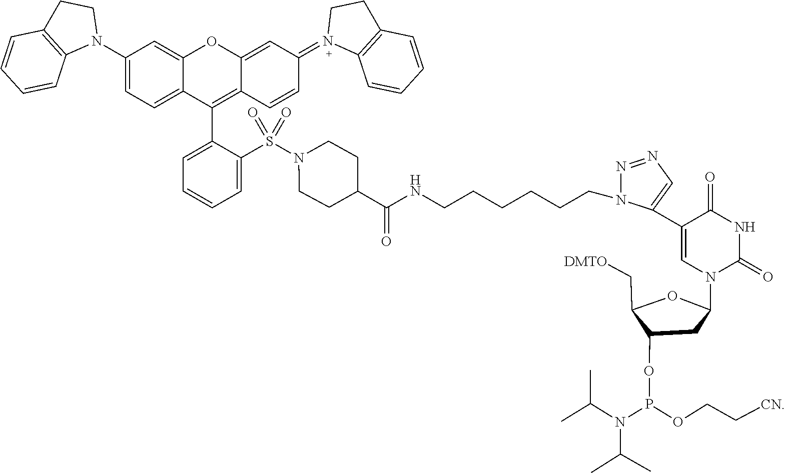

In embodiments, the compound is a compound as described herein, including in embodiments. In embodiments the compound is a compound described herein (e.g., in the examples section, figures, tables, or claims).

### Additional Label/Dye Details

Exemplary nonlimiting detectable labels that may be utilized with the embodiments described herein include, for example:
Fluoresceins (e.g., 5-carboxy-2,7-dichlorofluorescein, 5-Carboxyfluorescein (5-FAM), 6-JOE, 6-carboxyfluorescein (6-FAM), VIC, FITC, 6-carboxy-4′,5′-dichloro-2′,7′-dimethoxy-fluorescein (JOE)), 5 and 6-carboxy-1,4-dichloro-2′,7′-dichloro-fluorescein (TET), 5 and 6-carboxy-1,4-dichloro-2′,4′,5′,7′-tetra-chlorofluorescein, HEX, PET, NED, Oregon Green (e.g. 488, 500, 514));
Pyrenes (e.g. Cascade Blue; Alexa Fluor 405);
Coumarins (e.g. Pacific Blue, Atto 425, Alexa Fluor 350, Alexa Fluor 430);
Cyanine Dyes (e.g. Cy dyes such as Cy3, Cy3.18, Cy3.5, Cy5, Cy5.18, Cy5.5, Cy7);
Rhodamines (e.g., 110, 123, B, B 200, BB, BG, B extra, 5 and 6-carboxytetramethylrhodamine (5-TAMRA, 6-TAMRA), 5 and 6-Carboxyrhodamine 6G, Lissamine, Lissamine Rhodamine B, Rhod-2, ROX (6-carboxy-X-rhodamine), 5 and 6-ROX (carboxy-X-rhodamine), Sulphorhodamine B can C, Sulphorhodamine G Extra, 5 and 6 TAMRA (6-carboxytetramethyl-rhodamine), (TRITC), ABY, JUN, LIZ, RAD, RXJ, Texas Red; and Texas Red-X);
Alexa Fluor fluorophores (which is a broad class including many dye types such as cyanines) (e.g., Alexa 350, 405, 430, 488, 500, 514, 532, 546, 555, 568, 594, 610, 633, 635, 647, 660, 676, 680, 700, 750);
FRET donor/acceptor pairs (e.g., fluorescein/fluorescein, fluorescein/rhodamine, fluorescein/cyanine, rhodamine/cyanine, fluorescein/Alexa Fluor, Alexa Fluor/rhodamine); and other types of dyes known to those of skill in the art.

Fluorophore labels may be associated with quenchers such as dark fluorescent quencher (DFQ), black hole quenchers (BHQ), Iowa Black, QSY7, QSY21 quencher, Dabsyl and Dabcel sulfonate/carboxylate quenchers, and MGB-NFQ quenchers. Fluorophore labels may also include sulfonate derivatives of fluorescein dyes with SO3 instead of the carboxylate group, phosphoramidite forms of fluorescein, and/or phosphoramidite forms of Cy5, for example.

### Additional Amplification Details

Amplified products resulting from use of one or more embodiments described herein can be generated, detected, and/or analyzed on any suitable platform. In some embodiments, the nucleic acid targets may be single-stranded, double-stranded, or any other nucleic acid molecule of any size or conformation. The amplification processes described herein can include PCR (see, e.g., U.S. Pat. No. 4,683,202). In some embodiments, the PCR is real-time or quantitative PCR (qPCR). In some embodiments, the PCR is an end-point PCR. In some embodiments, the PCR is digital PCR (dPCR).

In some embodiments, the amplification process includes reverse transcription PCR (RT-PCR). A disclosed method may include, for example, subjecting the target nucleic acid to a reverse transcription reaction prior to amplification via PCR. In some embodiments, the amplification process includes one-step RT-PCR (e.g., in a single vessel or reaction volume) in which one or more reverse transcriptases are used in combination with one or more DNA polymerases.

Optionally, certain qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N₈₀₁₀₅₆₀). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR.

Other amplification methods, such as, e.g., loop-mediated isothermal amplification (“LAMP”), and other isothermal methods are also contemplated for use with the assay embodiments described herein.

The components described herein for enabling multiplexing utilizing probes with detectable labels having spectral similarity may be provided in a kit along with one or more additional components to enable an amplification process. Such components can include, for example, dNTPs, DNA polymerase, amplification buffers/reagents, master mix components as known in the art, and other components known in the art for enabling or assisting nucleic acid amplification.

### Computer System Implementation

In some embodiments, at least a portion of the methods described herein may be implemented using one or more computer systems. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

In an example embodiment, a computer system comprises one or more processors, and a memory storing one or more instructions which, when executed by the one or more processors, cause the one or more processors to perform a process of: obtaining, at multiple time points during one or more cycles of an amplification process, emission (e.g., fluorescence) signal data associated with a composite emission (e.g., fluorescence) signal from at least a first probe type comprising a first detectable label (e.g., fluorophore) and a second probe type comprising a second detectable label (e.g., fluorophore) which has spectral similarity with the first detectable label (e.g., fluorophore) and/or generates an identical or substantially identical signal, said first probe type and said second probe type differing in thermal and/or temporal properties; and determining, based at least partially on said emission signal data associated with said composite emission signal and thermal and/or temporal properties of one or more of said at least said first probe type and said second probe type, emission signal data associated with a emission signal from a given probe type of said at least said first probe type and said second probe type during said one or more cycles of said amplification process.

In some embodiments, utilizing the emission signal data associated with the composite emission signal and the first emission signal data as inputs for generating the emission signal data associated with the emission signal from the given probe type comprises: generating transformed first emission signal data by applying a transformation (e.g., linear) to the first emission signal data; and modifying the emission signal data associated with the composite emission signal with the transformed first emission signal data to generate the emission signal data associated with the emission signal from the given probe type.

In some embodiments, the one or more instructions, when executed by the one or more processors, further cause the one or more processors to perform a process of: quantifying a first target associated with the first probe type based upon at least the first emission signal data; and quantifying a second target associated with the second probe type based upon at least the generated emission signal data associated with the emission signal from the given probe type.

Some embodiments include one or more computer-readable media storing one or more instructions which, when executed by one or more processors of at least one computing device, cause the one or more processors to perform the foregoing process or other computer-implemented process as described herein.

Systems for implementing the disclosed embodiments may include various components, such as, by way of non-limiting example, processor(s), storage, sensor(s), I/O system(s), communication system(s), and the like. The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

Furthermore, a system may comprise or be in communication with I/O system(s). I/O system(s) may include any type of input or output device such as, by way of non-limiting example, a touch screen, a mouse, a keyboard, a controller, a speaker and/or others, without limitation. For example, the I/O system(s) may include a display system that may comprise any number of display panels, optics, laser scanning display assemblies, and/or other components.

Disclosed embodiments may also include physical and other computer-readable media for carrying or storing computer-executable instructions and/or data structures. Such computer-readable media can be any available media that can be accessed by a general-purpose or special-purpose computer system. Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

Those skilled in the art will appreciate that various embodiments may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. Embodiments may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component.” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

### EXAMPLES

FIG. 6A illustrates the fluorescence signal over cycle number measured at the annealing/extension temperature (65° C. in this example) and at the denaturation temperature (95° C. in this example) with TaqMan probe and EF probe compositions.

FIGS. 6B-6D illustrate results of qPCR duplex assay tests, measuring fluorescence signal in the FAM detection channel, in which TaqMan and EF probes were designed to generate fluorescence signals having spectral similarity (e.g., and may be detectable in the same detection channel) (FIG. 6B) or without having spectral similarity (e.g., and detectable in different detection channels) (FIGS. 6C and 6D). In the assay shown in FIG. 6B, both the TaqMan probes and the EF probes were labelled with FAM. In the assay shown in FIG. 6C, the TaqMan probes were labelled with ARY and the EF probes were labelled with FAM. In the assay shown in FIG. 6D, the TaqMan probes were labelled with FAM and the EF probes were labelled with ABY. The reaction mixture composition, template DNA concentrations, and amplification conditions were otherwise held the same across each assay.

In FIG. 6B, the top row shows the FAM channel fluorescence signal over cycle number measured at the denaturation temperature (95° C. in this example). This signal is expected to include fluorescence generated mostly by TaqMan probe labels (those that have been cleaved from the probes). The second row shows the fluorescence signal over cycle number measured at the denaturation temperature (95° C. in this example) and modified by a linear function that correlates the 95° C. measurement to a 65° C. measurement for the TaqMan probes. This signal is expected to include fluorescence generated by the TaqMan probe labels but not to include significant fluorescence from the EF probe labels. The third row shows the fluorescence signal over cycle number measured at the annealing/extension temperature (65° C. in this example). This signal is expected to include fluorescence generated by both the TaqMan probe labels (those that have been cleaved from the probes) and the EF probe labels (those that have been incorporated into double-stranded amplicons). The bottom row shows the resolved fluorescence signal determined by subtracting the second row signal from the third row signal. This signal is expected to estimate the fluorescence generated by the EF probe labels, separate from fluorescence attributable to the TaqMan probe labels.

In FIG. 6C, the top, second, third, and bottom rows represent the same signal measurement types as in FIG. 6B with TaqMan probe labelled with ARY and EF probe labelled with FAM. As shown on the top and second rows, the EF probe labels (FAM) generated insignificant (essentially baseline level) fluorescence in FAM channel at the denaturation temperature. Because the TaqMan and EF probes were differentially labelled in this assay, the bottom row shows a resolved signal for the EF probe label that essentially matches the EF probe signal at the annealing/extension temperature in FAM channel (third row).

In FIG. 6D, the top, second, third, and bottom rows represent the same signal measurement types as in FIG. 6B but with TaqMan probe labelled with FAM and EF probe labelled with ABY. The first-row fluorescence signal is mostly generated by the TaqMan probe label (those that have been cleaved from the probes). The second row shows the fluorescence signal over cycle number measured at the denaturation temperature (95° C. in this example) and modified by a linear function that correlates the 95° C. measurement to a 65° measurement for the TaqMan probes. Because the TaqMan and EF probes were differentially labelled in this assay, the derived TaqMan signal in FAM channel (second row) essentially matches the measured signal at the annealing/extension temperature in FAM channel (third row), and the resolved signal for EF probe label in FAM channel (bottom row) is essentially zero.

While FIGS. 6B-6D show results from a qPCR assay, the same principles illustrated can be extended to an end-point PCR assay with similar results. Specifically, a correction factor can be applied to signal measured at the denaturation set of conditions (e.g., denaturation temperature); the derived signal can be subtracted from measured signal at extension set of conditions (e.g., extension temperature) to obtain derived signal from the second probe (non-cleavable), with the measure signal from the denaturation set of conditions representing signal from first probe (cleavable probe).

FIG. 6E compares the resolved EF-associated fluorescence signal after baseline adjustment (ΔRn) (bottom row of FIG. 6B) with the EF-associated fluorescence signal after baseline adjustment (ΔRn) (bottom row of FIG. 6C), which represents a direct measurement of EF probe label fluorescence in FAM channel. The results showed close correlation between the resolved and measured signals. The results therefore showed that fluorescent signals attributable to different probe types within the same detection channel can be separately resolved.

FIG. 6F compares the derived TaqMan-associated fluorescence signal after baseline adjustment (ΔRn) (second row of FIG. 6B) with the derived TaqMan-associated fluorescence signal after baseline adjustment (ΔRn) (second row of FIG. 6D) in FAM channel. The results showed close correlation between the derived TaqMan signals from separate assays where the EF probes are similarly labelled (FIG. 6B) or labelled with a different dye (FIG. 6D).

FIG. 7 illustrates the results of another assay test that included 5 different detection channels/dyes, four detection channels with a corresponding TaqMan probe and an EF probe (with each channel having a differing dye common to the TaqMan and EF probes in that channel), and one channel with only a TaqMan probe (labeled AF647 in the index of FIG. 7). The results show that the fluorescent signals of the different probe types can be independently determined, and that a 9-plex reaction can be effectively carried out utilizing 5 detection channels.

FIG. 8 is a plot comparing the end-point signals of reaction volumes at 65° C. and at 95° C. following a dPCR process. As shown, the signals fall into identifiable clusters. The clusters may be estimated using cluster analysis algorithms known in the art. In FIG. 8, the “EF” cluster represents those partitions that provide a signal at the annealing/extension temperature but have limited signal at the denaturation temperature, the “T” cluster represents those partitions that provide a signal at both the annealing/extension temperature and the denaturation temperature, and the “T+EF” cluster represents those partitions that provide a signal at the denaturation temperature and a heightened signal at the annealing/extension temperature. The total count of partitions in which the TaqMan probes generated a signal equals the count of cluster T added to the count of cluster T+EF, and the total count of partitions in which the EF probes generated a signal equals the count of cluster EF added to the count of cluster T+EF. Concentrations of the first and second target in the sample may then be estimated using standard dPCR techniques.

The cluster analysis technique of FIG. 8 is one embodiment of a technique for analyzing signals obtained in a dPCR process. For other data analysis techniques that can be used to resolve the signal data obtained in a multiplex dPCR process utilizing detectable labels having spectral similarity in accordance with the present disclosure.

The present disclosure includes, but is not limited to, embodiments represented by the following clauses.
Clause 1: A method of detecting nucleic acids in a sample, comprising:
(i) providing a reaction mixture, the reaction mixture including
   at least a portion of the sample,
   a first probe detectably labeled with a first label configured to generate a first emission signal,
   a second probe detectably labeled with a second label configured to generate a second emission signal,
   wherein the first and second probes have different sequences, and
   wherein the first and second labels are identical and/or generate substantially identical emission;
(ii) subjecting the reaction mixture to an amplification process comprising a first set of reaction conditions and a second set of reaction conditions, the first set of reaction conditions being different than the second set of reaction conditions; and
(iii) determining the presence of the first and/or second nucleic acid targets in the sample by
   measuring an emission signal during the first set of reaction conditions, the emission signal during the first set of reaction conditions being correlated with specific interaction or lack of interaction between the first probe and a first nucleic acid target,
   measuring an emission signal during the second set of reaction conditions, the emission signal during the second set of reaction conditions being correlated with specific interaction or lack of interaction between the first probe and a first nucleic acid target and with specific or lack of interaction between the second probe and a second nucleic acid target, and
   estimating the presence and/or amount of each of the first nucleic acid target and second nucleic acid target.
Clause 2: A method of detecting nucleic acids in a sample, comprising:
(i) providing a reaction mixture, the reaction mixture including:
   at least a portion of the sample,
   a first probe detectably labeled with a first label configured to generate a first emission signal that is indicative of the presence or absence of a first nucleic acid target,
   a second probe detectably labeled with a second label configured to generate a second emission signal that is indicative of the presence or absence of a second nucleic acid target,
   wherein the first and second probes have different sequences, and
   wherein the first and second labels are identical and/or generate substantially identical emission signals;
(ii) subjecting the reaction mixture to an amplification process comprising a first set of reaction conditions and a second set of reaction conditions, the first set of reaction conditions being different than the second set of reaction conditions; and
(iii) determining any presence and/or amount of each of the first and/or second nucleic acid targets in the reaction mixture by
   measuring during the first set of reaction conditions a first total emission signal that comprises any first emission signal if present and any second emission signal if present,
   measuring during the second set of reaction conditions a second total emission signal comprising any first emission signal if present and any second emission signal if present, and
   estimating the first emission signal and/or second emission signal based on the first and second total emission signals.
Clause 3: The method of Clause 1 or 2, wherein the first and second emission signals are first and second fluorescence signals, and wherein both the first and second probes are subjected to excitation at the same wavelength and/or both the first and second probes are subjected to excitation during detection of their respective first and second fluorescence signals.
Clause 4: The method of any one of Clauses 1-3, wherein the emission signals are fluorescence signals and wherein:
measuring the fluorescence signal during the first set of reaction conditions comprises measuring a combined signal comprising the first and second fluorescence signals during the first set of reaction conditions to obtain a first total fluorescence signal;
measuring the fluorescence signal during the second set of reaction conditions comprises measuring a combined signal comprising the first and second fluorescence signals during the second set of reaction conditions to obtain a second total fluorescence signal; and
estimating the presence and/or amount of each of the first nucleic acid target and the second nucleic acid target comprises estimating the first fluorescence signal and/or the second fluorescence signal based on the first and second total fluorescence signals.
Clause 5: The method of Clause 4, wherein the second fluorescence signal differs between the first and second set of reaction conditions to a greater degree than the first fluorescence signal differs between the first and second set of reaction conditions.
Clause 6: The method of any one of Clauses 4-5, wherein
the first total fluorescence value comprises (i) fluorescence from first label that is free and unquenched within the reaction mixture and emitted as a result of the first label being cleaved following hybridization of the first probe to the first amplicon, and (ii) background fluorescence of the second label, and
the second total fluorescence value is based on (i) fluorescence from first label that is free and unquenched within the reaction mixture and emitted as a result of the first label being cleaved following hybridization of the first probe to the first amplicon, and (ii) fluorescence from the second label, above the background fluorescence of the second label, emitted as a result of hybridization of the second probe to the second amplicon.
Clause 7: The method of any one of Clauses 4-6, further comprising:
calculating an amount of the first nucleic acid target based on the first fluorescent signal; and
calculating an amount of the second nucleic acid target based on the second fluorescent signal.
Clause 8: The method of any one of Clause 4-7, wherein the first fluorescent signal being above a background level during both the first and second sets of reaction conditions indicates presence of the first nucleic acid target in the reaction mixture.
Clause 9: The method of any one of Clauses 4-8, wherein the second fluorescent signal being above a background level during the second set of reaction conditions but not during the first set of reaction conditions indicates presence of the second nucleic acid target in the reaction mixture.
Clause 10: The method of any one of Clauses 5-8, wherein the first set of reaction conditions comprises a first measurement temperature at which the first fluorescence signal is measured, and the second set of reaction conditions comprises a second measurement temperature at which the second fluorescence signal is measured, the second measurement temperature being different than the first measurement temperature.
Clause 11: The method of Clause 10, wherein the first and second measurement temperatures differ by at least about 10° C. or more, about 15° C. or more, about 20° C. or more, about 25° C. or more, or about 30° C. or more.
Clause 12: The method of Clause 10 or Clause 11, wherein at least one of the first or second measurement temperatures is a denaturation temperature at which DNA in the reaction mixture is denatured, such as in a range of about 80° C. or above.
Clause 13: The method of any one of Clauses 1-12, wherein the reaction mixture is subjected to multiple amplification cycles during the amplification process, each of the amplification cycles comprising the first and second set of reaction conditions.
Clause 14: The method of any one of Clauses 1-13, wherein the amplification process comprises thermal cycling.
Clause 15: The method of Clause 14, wherein the subjecting the reaction mixture to the first set of reaction conditions comprises thermal cycling the reaction mixture at a first temperature sufficient to cause denaturation of the first and second amplicons.
Clause 16: The method of Clause 15, wherein the subjecting the reaction mixture to the second set of reaction conditions comprises thermal cycling the reaction mixture at a second temperature sufficient to cause annealing and/or extension of the first nucleic acid target and the second nucleic acid target to respectively form the first amplicon and the second amplicon, the second temperature being lower than the first temperature.
Clause 17: The method of any one of Clauses 1-16 wherein the first probe is a cleavable probe.
Clause 18: The method of Clause 17, wherein the first emission signal increases as the cleavable probe is cleaved during an annealing/extension stage.
Clause 19: The method of Clauses 17 or 18, wherein the first probe includes a fluorophore and a quencher, and wherein the first probe is configured such that fluorescence from the fluorophore is quenched by the quencher until the probe is cleaved during an annealing/extension stage of the amplification process.
Clause 20: The method of Clause 19, wherein the first probe is a TaqMan probe.
Clause 21: The method of any one of Clauses 1-20, wherein the second probe is a non-cleavable probe.
Clause 22: The method of Clause 21, wherein the second probe comprises a stem-loop portion configured to form a stem-loop structure when the second probe is single-stranded.
Clause 23: The method of Clauses 21 or 22, wherein the second probe comprises a fluorophore and a quencher spaced apart from one another such that the fluorophore is quenched when the second probe is single-stranded and unquenched when the second probe is incorporated into a double-stranded amplicon.
Clause 24: The method of Clause 23, wherein the fluorophore is located at or near the 5′ end of the second probe and the quencher is 3′ of the fluorophore.
Clause 25: The method of Clauses 23 or 24, wherein both the fluorophore and the quencher are disposed at or near the stem loop portion of the second probe.
Clause 26: The method of any one of Clauses 1-25, wherein the reaction mixture further comprises: a first primer pair complementary to a first nucleic acid target of the nucleic acids or its complement, the first nucleic acid target being configured to generate a first amplicon with which the first probe can hybridize; and a second primer pair complementary to a second nucleic acid target of the nucleic acids or its complement, the second nucleic acid target being configured to generate a second amplicon with which the second probe can hybridize.
Clause 27: The method of Clause 26, wherein the second primer pair includes a primer with a tail.
Clause 28: The method of Clause 27, wherein the tail forms the 5′ end of the primer with the tail.
Clause 29: The method of Clause 27 or Clause 28, wherein the second probe can hybridize to the tail or to its complement.
Clause 30: The method of any one of Clauses 20-29, wherein the amplification process utilizes a series of thermal cycling stages that includes at least three different target temperatures.
Clause 31: The method of Clause 30, wherein the amplification process includes a denaturation temperature and multiple different annealing/extension temperatures that vary throughout the amplification process.
Clause 32: The method of Clause 31, wherein a first series of denaturation and annealing/extension stages are carried out at a first annealing/extension temperature, and wherein a second series of denaturation and annealing/extension stages are carried out at a second annealing/extension temperature different from the first annealing/extension temperature.
Clause 33: The method of Clause 32, wherein the first annealing/extension temperature is higher than the second annealing/extension temperature.
Clause 34: The method of Clause 32 or Clause 33, wherein the first series of denaturation and annealing/extension stages are cycled a greater number of times than the second series of denaturation and annealing/extension stages.
Clause 35: The method of any one of Clauses 32-34, wherein the amplification process further comprises a third series of denaturation and annealing/extension steps carried out using a third annealing/extension temperature.
Clause 36: The method of Clause 35, wherein the third annealing/extension temperature is the same as the first annealing/extension temperature.
Clause 37: The method of Clause 35 or Clause 36, wherein the third series of denaturation and annealing/extension stages are cycled a greater number of times than the first series of denaturation and annealing/extension stages.
Clause 38: The method of any one of Clauses 32-37, wherein the denaturation temperature is the same for each series of the denaturation stages.
Clause 39: The method of any one of Clauses 2738, wherein the second primer pair further includes a non-tailed primer, and wherein a concentration of the primer with the tail in the reaction mixture is different from that of the non-tailed primer in the reaction mixture.
Clause 40: The method of Clause 39, wherein the concentration of the non-tailed primer is greater than that of the primer with the tail.
Clause 41: The method of Clause 40, wherein the concentration of the non-tailed primer is about 2× to about 30× greater than the concentration of the primer with the tail, or about 5× to about 25× greater than the concentration of the primer with the tail, or about 10× to about 20× greater than the concentration of the primer with the tail.
Clause 42: The method of any one of Clauses 39-41, wherein the second probe is provided at a concentration that is different from the concentration of the primer with the tail and the concentration of the non-tailed primer.
Clause 43: The method of Clause 42, wherein the second probe is provided at a concentration that is greater than the concentration of the primer with the tail.
Clause 44: The method of Clause 42 or Clause 43, wherein the second probe is provided at a concentration that is less than the concentration of the non-tailed primer.
Clause 45: The method of any one of Clauses 42-44, wherein the second probe is provided at a concentration that is about 2× to about 10× the concentration of the primer with the tail, or about 3× to about 7.5× the concentration of the primer with the tail.
Clause 46: The method of any one of Clauses 1-45, wherein a melting temperature (Tₘ) of the first probe and a Tₘ of the second probe are within about 8° C., or about 6° C., or about 4° C., or about 2° C. of each other.
Clause 47: The method of any one of Clauses 1-48, wherein the amplification process cycles between at least two target temperatures for multiple cycles of the amplification process.
Clause 48: The method of Clause 47, wherein the amplification process cycles between at least two target temperatures for at least 5% of, at least 10% of, at least 15% of, at least 20% of, at least 25% of, at least 30% of, at least 35% of, at least 40%, of, at least 45% of, at least 50% of, at least 55% of, at least 60% of, at least 65% of, at least 70% of, at least 75% of, at least 80% of, at least 85% of, at least 90% of, or at least 95% of the cycles of the amplification process.
Clause 49: The method of any one of Clauses 1-48, wherein the method further comprises partitioning the reaction mixture into a plurality of reaction volumes, and wherein the amplification process is a digital PCR (dPCR) process.
Clause 50: The method of Clause 49, wherein measuring the emission signal during the first set of reaction conditions comprises measuring the emission signal upon or near completion of the subjecting the reaction mixture to the first set of reaction conditions to obtain a first end-point measurement, and wherein measuring the emission signal during the second set of reaction conditions comprises measuring the emission signal upon or near completion of the subjecting the reaction mixture to the second set of reaction conditions to obtain a second end-point measurement.
Clause 51: The method of Clause 50, wherein the estimating the presence and/or amount of each of the first nucleic acid target and the second nucleic acid target comprises:
categorizing the plurality of reaction volumes according to the emission signal measured at the first end-point measurement and according to the emission signal measured at the second end-point measurement; and
based on the categorizations, determining a count for the plurality of reaction volumes in which the first probe showed activity and a count for the plurality of reaction volumes in which the second probe showed activity.
Clause 52: The method of any one of Clauses 1-48, wherein the measuring the emission signal during the first set of reaction conditions comprises measuring the emission signal during a denaturation stage of an end-point cycle of the amplification process, and wherein the measuring the emission signal during the second set of reaction conditions comprises measuring the emission signal during an annealing and/or extension state of the end-point cycle of the amplification process.
Clause 53: The method of any one of Clauses 1-48 or 52, wherein the amplification process is an end-point PCR process.
Clause 54: A method of detecting nucleic acids in a sample, comprising:
providing a reaction mixture, the reaction mixture comprising:
   a primer pair complementary to a nucleic acid target or its complement for generating an amplicon, and
   a non-cleavable probe configured to hybridize to the amplicon, the non-cleavable probe including a detectable label configured to provide an emission signal that corresponds to an amount of generated amplicon,
subjecting the reaction mixture to an amplification process to generate the amplicons, wherein the label generates emission without cleavage of the non-cleavable probe during the amplification process, and wherein the amplification process utilizes a series of thermal cycling stages that includes at least three different target temperatures; and
measuring the emission signal from the non-cleavable probe.
Clause 55: The method of Clause 54, further comprising quantitating an amount of the nucleic acid target based on the measured emission signal.
Clause 56: The method of Clause 54 or Clause 55, wherein the non-cleavable probe comprises a stem-loop portion capable of forming a stem-loop structure when the non-cleavable probe is single-stranded.
Clause 57: The method of any one of Clauses 54-56, wherein the non-cleavable probe comprises a fluorophore and a quencher spaced such that the fluorophore is quenched when the non-cleavable probe is single-stranded but enabled when the probe is incorporated into a double-stranded amplicon.
Clause 58: The method of Clause 57, wherein the fluorophore is located at or near the 5′ end of the probe and the quencher is 3′ of the fluorophore.
Clause 59: The method of Clause 57 or Clause 58, wherein both the fluorophore and the quencher are at or near the stem loop portion of the probe.
Clause 60: The method of any one of Clauses 54-59, wherein the primer pair includes a primer with a tail.
Clause 61: The method of Clause 60, wherein the tail forms the 5′ end of the primer with the tail.
Clause 62: The method of Clause 60 or cl Clause aim 61, wherein the non-cleavable probe is configured to hybridize to the tail or to its complement.
Clause 63: The method of Clause 62, wherein a 3′ portion of the non-cleavable probe is configured to hybridize to the tail or its complement.
Clause 64: The method of any one of Clauses 54-63, wherein the amplification process includes a denaturation temperature and multiple different annealing/extension temperatures that vary throughout the amplification process.
Clause 65: The method of Clause 64, wherein a first series of denaturation and annealing/extension stages are carried out at a first annealing/extension temperature, and wherein a second series of denaturation and annealing/extension stages are carried out at a second annealing/extension temperature that is different from the first annealing/extension temperature.
Clause 66: The method of Clause 65, wherein the first annealing/extension temperature is higher than the second annealing/extension temperature.
Clause 67: The method of Clause 65 or Clause 66, wherein the first series of denaturation and annealing/extension stages are cycled a greater number of times than the second series of denaturation and annealing/extension stages.
Clause 68: The method of any one of Clause 65-67, wherein the amplification process further comprises a third series of denaturation and annealing/extension stages carried out using a third annealing/extension temperature.
Clause 69: The method of Clause 68, wherein the third annealing/extension temperature is the same as the first annealing/extension temperature.
Clause 70: The method of Clause 68 or Clause 69, wherein the third series of denaturation and annealing/extension stages are cycled a greater number of times than the first series of denaturation and annealing/extension stages.
Clause 71: The method of any one of Clauses 65-70, wherein the denaturation temperature is the same for each series of denaturation stages.
Clause 72: The method of any one of Clauses 65-71, wherein the primer pair further includes a non-tailed primer, and wherein a concentration of the primer with the tail in the reaction mixture is different than that of the non-tailed primer.
Clause 73: The method of Clause 72, wherein the non-tailed primer is provided at a greater concentration than the primer with the tail.
Clause 74: The method of Clause 73, wherein the non-tailed primer is provided at a concentration that is about 2× to about 30× the concentration of the primer with the tail, or about 5× to about 25× the concentration of the primer with the tail, or about 10× to about 20× the concentration of the primer with the tail.
Clause 75: The method of any one of Clauses 74-74, wherein a concentration of the non-cleavable probe in the reaction mixture is different than a concentration of the primer with the tail and a concentration of the non-tailed primer in the reaction mixture.
Clause 76: The method of Clause 75, wherein a concentration of the non-cleavable probe in the reaction mixture is greater than a concentration of the primer with the tail in the reaction mixture.
Clause 77: The method of Clause 75 or Clause 76, wherein the non-cleavable probe is provided at a concentration that is less than the concentration of the non-tailed primer.
Clause 78: The method of any one of Clauses 77-77, wherein the non-cleavable probe is provided at a concentration that is about 2× to about 10× the concentration of the non-tailed primer, or about 3× to about 7.5× the concentration of the non-tailed primer.
Clause 79: The method of any one of Clauses 54 or 55,
wherein the reaction mixture further comprises:
   a primer pair complementary to a second nucleic acid target or its complement for generating a second amplicon, and
   a cleavable probe configured to hybridize to the second amplicon, the cleavable probe including a detectable label configured to provide an emission signal that corresponds to an amount of generated second amplicon;
wherein the subjecting the reaction mixture to the amplification process generates second amplicons, wherein the detectable label of the cleavable probe generates emission due to cleavage of the cleavable probe during the amplification process; and
wherein the method further comprises measuring the emission signal from the cleavable probe.
Clause 80: The method of Clause 81, further comprising quantitating an amount of the second nucleic acid target based on the measured emission signals.
Clause 81: A method of detecting nucleic acids in a sample, comprising:
providing a reaction mixture, the reaction mixture comprising:
   a primer pair targeted to a nucleic acid target for generating an amplicon, the primer pair including a primer with the tail and a non-tailed primer provided at different concentrations, and
   a detectably labelled, non-cleavable probe configured to hybridize to the amplicon and to generate a fluorescent signal that corresponds to an amount of generated amplicon,
subjecting the reaction mixture to an amplification process to generate the amplicons, wherein the non-cleavable probe generates emission without being cleaved during the amplification process; and
measuring the emission signal from the non-cleavable probe.
Clause 82: The method of Clause 81 wherein the non-tailed primer is provided at a greater concentration than the primer with the tail.
Clause 83: The method of Clause 82, wherein the non-tailed primer is provided at a concentration that is about 2× to about 30× the concentration of the primer with the tail, or about 5× to about 25× the concentration of the primer with the tail, or about 10× to about 20× the concentration of the primer with the tail.
Clause 84: The method of any one of Clause 80-83, wherein the non-cleavable probe is provided at a concentration that is different from the concentration of the primer with the tail and the concentration of the non-tailed primer.
Clause 85: The method of Clause 84, wherein the non-cleavable probe is provided at a concentration that is greater than the concentration of the primer with the tail.
Clause 86: The method of Clause 84 or Clause 84, wherein the non-cleavable probe is provided at a concentration that is less than the concentration of the non-tailed primer.
Clause 87: The method of any one of Clauses 84-86, wherein the non-cleavable probe is provided at a concentration that is about 2× to about 10× the concentration of the non-tailed primer, or about 3× to about 7.5× the concentration of the non-tailed primer.
Clause 88: The method of Clause 81,
wherein the reaction mixture further comprises:
   a primer pair targeted to a second nucleic acid target different from the nucleic acid target and for generating a second amplicon, the primer pair including a primer with the tail and a non-tailed primer provided at different concentrations, and
   a detectably labelled, cleavable probe configured to hybridize to the second amplicon and to generate an emission signal that corresponds to an amount of generated second amplicon;
wherein subjecting the reaction mixture to an amplification process generates the second amplicons, wherein the cleavable probe generates emission signal without due to cleavage during the amplification process; and
wherein the method further comprises measuring the emission signal from the cleavable probe.
Clause 89: A method of detecting the presence or amount of a first and/or second target in a reaction mixture, comprising:
including a first and second probe in the reaction mixture, wherein the first probe can specifically interact with a first target and comprises a first label that can produce a first detectable signal, and the second probe can specifically interact with a second target and comprises a second label that can produce a second detectable signal;
allowing specific interaction of the first and second probe with any first and second target, respectively, in the reaction mixture;
measuring a first total signal through an optical filter under a first set of conditions, wherein the first total signal includes the first and second detectable signals from the first and second labels, and wherein under the first set of conditions, the first detectable signal is increased as a result of specific interaction of the first probe with the first target, but the second detectable signal is not increased as a result of specific interaction of the second probe with the second target;
measuring a second total signal through the same optical filter under a second set of conditions, wherein the second total signal includes the first and second detectable signals from the first and second labels, and wherein under the second set of conditions, the second detectable signal is increased as a result of specific interaction of the second probe with the second target; and
assessing the presence or amount of the first and/or second target, by estimating the first detectable signal and the second detectable signal based on both the first total signal and the second total signal.
Clause 90: The method of Clause 89, wherein the first and second labels are identical and/or generate substantially identical fluorescence.
Clause 91: The method of Clause 89 or Clause 90, wherein the second fluorescence signal differs between the first and second set of conditions to a greater degree than the first fluorescence signal differs between the first and second set of conditions.
Clause 92: The method of any one of Clauses 89-91, wherein the first probe is a cleavable probe.
Clause 93: The method of Clause 92, wherein the first detectable signal increasing indicates the cleavable probe is cleaved.
Clause 94: The method of Clause 92 or Clause 93, wherein the first probe includes a fluorophore and a quencher, and wherein the first probe is configured such that fluorescence from the fluorophore is quenched by the quencher until the probe is cleaved.
Clause 95: The method of Clause 94, wherein the first probe is a TaqMan probe.
Clause 96: The method of any one of Clauses 89-95, wherein the second probe is a non-cleavable probe.
Clause 97: The method of Clause 96, wherein the second probe comprises a stem-loop portion capable of forming a stem-loop structure when the second probe is single-stranded.
Clause 98: The method of Clause 96 or Clause 97, wherein the second label of the second probe is a fluorophore, wherein the second probe further comprises a quencher spaced such that the fluorophore is quenched when the second probe is single-stranded but enabled when the second probe is incorporated into a double-stranded nucleic acid.
Clause 99: The method of Clause 98, wherein the fluorophore is located at or near the 5′ end of the second probe and the quencher is 3′ of the fluorophore.
Clause 100: The method of Clause 98 or Clause 99, wherein both the fluorophore and the quencher are disposed at or near the stem loop portion of the second probe.
Clause 101: The method of any one of Clauses 89-100, wherein a melting temperature (Tₘ) of the first probe and a Tₘ of the second probe are within about 8° C., or about 6° C., or about 4° C., or about 2° C. of each other.
Clause 102: The method of any one of Clauses 89-101, wherein the first set of conditions comprises a first measurement temperature at which the first fluorescence signal is measured, and the second set of conditions comprises a second, different measurement temperature at which the second fluorescence signal is measured.
Clause 103: The method of Clause 102, wherein the first and second measurement temperatures differ by at least about 10° C. or more, about 15° C. or more, about 20° C. or more, about 25° C. or more, or about 30° C. or more.
Clause 104: The method of Clause 102 or Clause 103, wherein at least one of the first or second measurement temperatures is a denaturation temperature at which DNA in the reaction mixture is denatured, such as about 90° C. or above.
Clause 105: The method of any one of Clauses 89-104, further comprising thermal cycling of the reaction mixture between two target temperatures for multiple cycles.
Clause 106: The method of Clause 105, wherein the thermal cycling cycles between two target temperatures for at least 5% of, at least 10% of, at least 15% of, at least 20% of, at least 25% of, at least 30% of, at least 35% of, at least 40%, of, at least 45% of, at least 50% of, at least 55% of, at least 60% of, at least 65% of, at least 70% of, at least 75% of, at least 80% of, at least 85% of, at least 90% of, or at least 95% of the cycles.
Clause 107: The method of any one of cl Clauses aims 54-106, wherein measuring the signals occurs at an end-point thermal cycle of the amplification process.
Clause 108: The method of any one of Clauses 1, 2, or 86, wherein the first probe is configured to produce a cumulative signal across differing stages of a cycle of an amplification process and the second probe is configured to produce a transient signal during differing stages of a cycle of an amplification process.
Clause 109: The method of any one of Clauses 1-108, wherein the second probe is a compound having the formula:

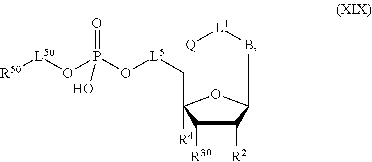

or a salt thereof, wherein
Q is an internal quencher moiety having the formula:

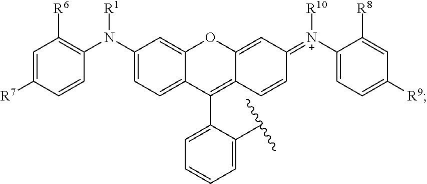

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted
heteroarylene;
R⁵⁰ is the second label or the detectable label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, □NHNH₂, □ONH₂, □NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃. —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂L, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Clause 110: The method of Clause 109, wherein L⁵ forms a stem-loop structure when the second probe is single-stranded.
Clause 111: The method of Clause 109, wherein R⁵⁰ is a fluorophore, and Q and R⁵⁰ are spaced apart from one another such that R⁵⁰ is quenched when the second probe is single stranded and unquenched when the second probe is incorporated into a double-stranded amplicon.
Clause 112: The method of Clause Error! Reference source not found., wherein both Q and R⁵⁰ are disposed at or near the stem loop portion of the second probe.
Clause 113: The method of Clause 109, wherein L⁵ comprises from 11 to 30 nucleotides.
Clause 114: The method of Clause 109, wherein L⁵ comprises from 19 to 23 nucleotides.
Clause 115: The method of Clause 109, wherein L⁵ comprises from 4 to 14 nucleotides.
Clause 116: The method of Clause 109, wherein L⁵ comprises from 6 to 12 nucleotides.
Clause 117: The method of Clause 109, wherein the nucleotides are DNA nucleotides.
Clause 118: The method of Clause 109, wherein the nucleotides are RNA molecules.
Clause 119: The method of Clause 109, wherein the compound has the formula:

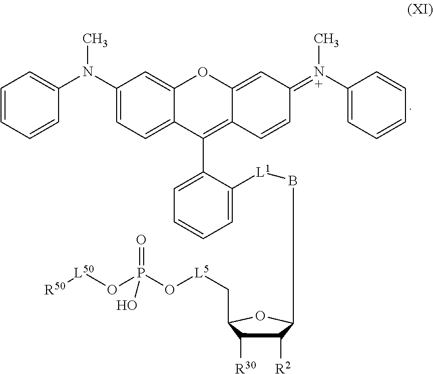

Clause 120: The method of Clause 109, wherein the compound has the formula:

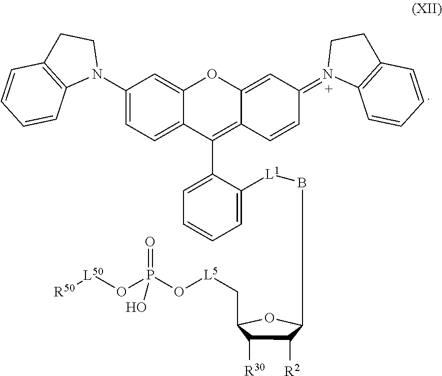

Clause 121: The method of Clause 109, wherein the compound has the formula:

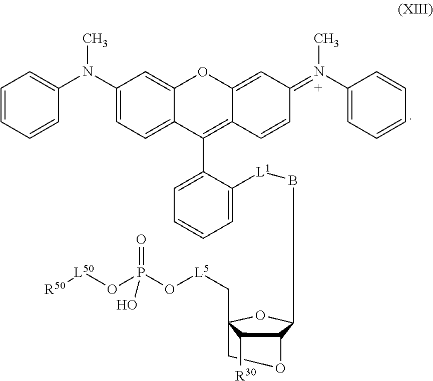

Clause 122: The method of Clause 109, wherein the compound has the formula:

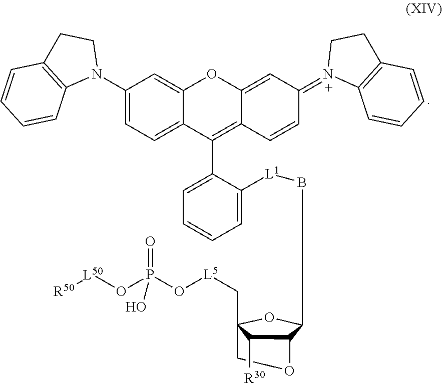

Clause 123: The method of Clause 109, wherein B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof.
Clause 124: The method of Clause 109, wherein B is a divalent cytosine or a derivative thereof, divalent guanine or a derivative thereof, divalent adenine or a derivative thereof, divalent thymine or a derivative thereof, or divalent uracil or a derivative thereof.
Clause 125: The method of Clause 109, wherein the compound has the formula:

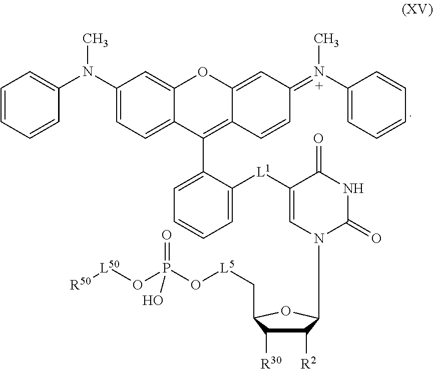

Clause 126: The method of Clause 109, wherein the compound has the formula:

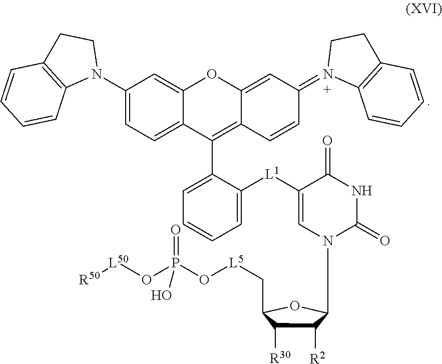

Clause 127: The method of Clause 109, wherein the compound has the formula:

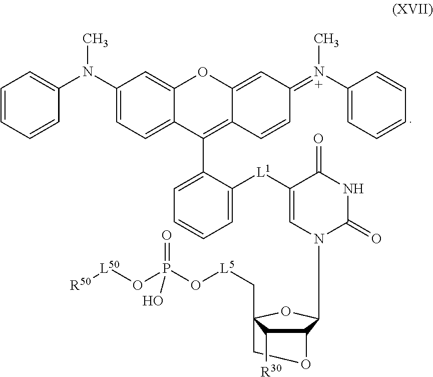

Clause 128: The method of Clause 109, wherein the compound has the formula:

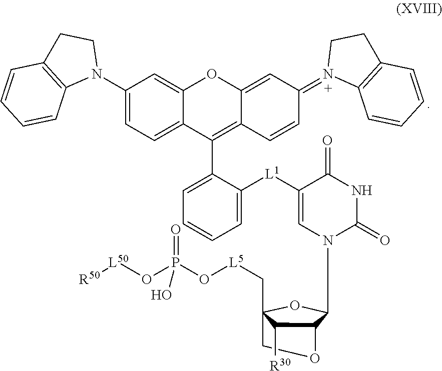

Clause 129: The method of Clause 109, wherein Li is L¹⁰¹-L¹⁰²-L¹⁰³-L¹⁰⁴-L¹⁰⁵; and L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
Clause 130: The method of Clause 129, wherein L¹⁰¹ is —S(O)₂—.
Clause 131: The method of Clause 129, wherein L¹⁰² is an unsubstituted 3 to 8 membered heterocycloalkyl.
Clause 132: The method of Clause 129, wherein L¹⁰² is an unsubstituted piperidinyl.

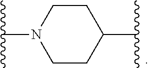 [SMILES: *C1CCN(*)CC1]

Clause 133: The method of Clause 129, wherein L¹⁰² is.
Clause 134: The method of Clause 129, wherein L¹⁰³ is —C(O)NH—.
Clause 135: The method of Clause 129, wherein L¹⁰⁴ is an unsubstituted C₁-C₁₀ alkylene, unsubstituted 2 to 6 membered heteroalkylene, or unsubstituted phenylene.
Clause 136: The method of Clause 129, wherein L¹⁰⁴ is an unsubstituted n-hexylene,

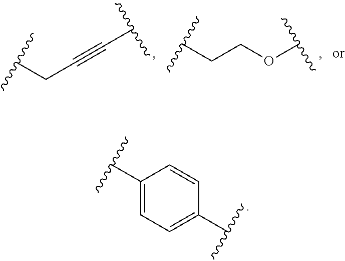 [SMILES: *C#CC* ; *CCO* ; *c1ccc(*)cc1]

Clause 137: The method of Clause 129, wherein L¹⁰⁵ is an unsubstituted C₁-C₁₀ alkylene, substituted or unsubstituted 2 to 8 membered heteroalkylene, or unsubstituted 5 to 10 membered heteroarylene.
Clause 138: The method of Clause 129, wherein L¹⁰⁵ is

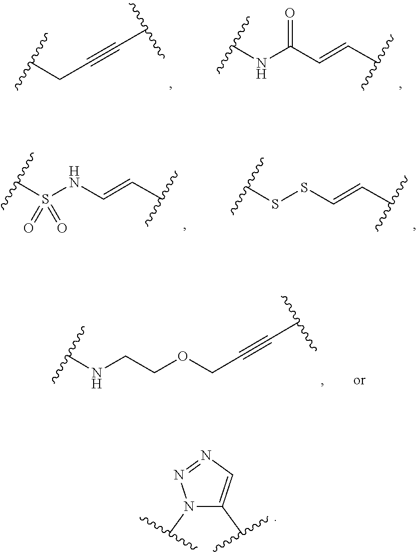 [SMILES: *C#CC* ; */C=C/C(=O)N* ; */C=C/NS(*)(=O)=O ; */C=C/SS* ; *C#CCOCCN* ; *c1cnnn1*]

Clause 139: The method of Clause 129, wherein L¹ is

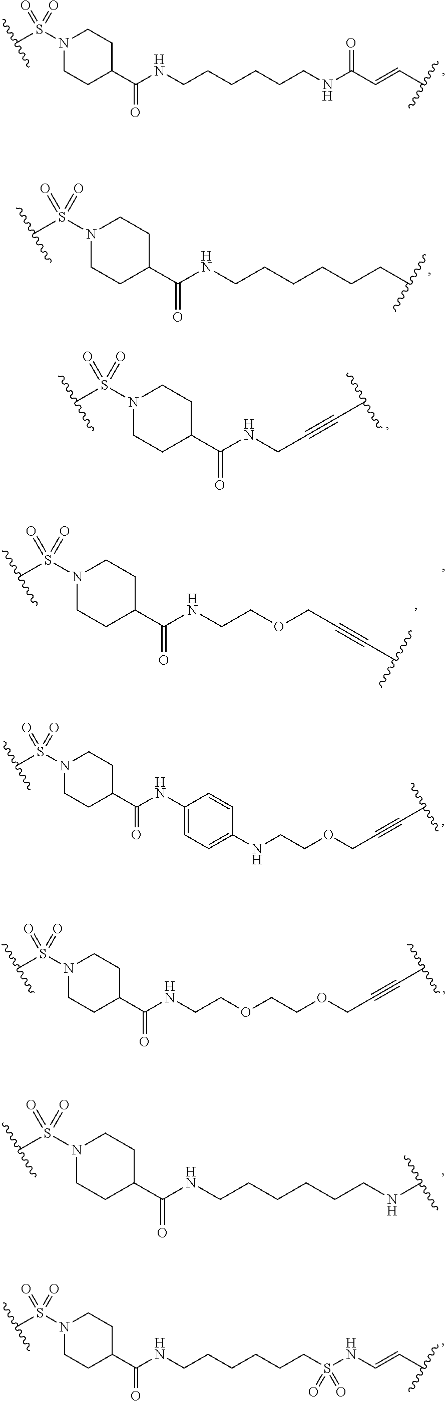

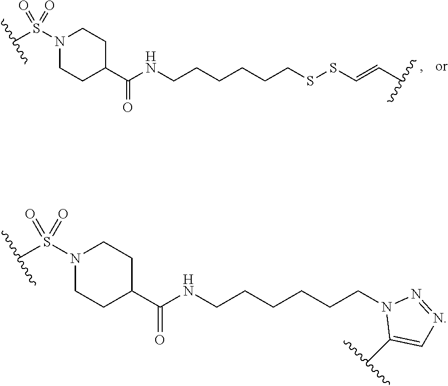 [SMILES: */C=C/SSCCCCCCNC(=O)C1CCN(S(*)(=O)=O)CC1 ; *c1cnnn1CCCCCCNC(=O)C1CCN(S(*)(=O)=O)CC1]

Clause 140: The method of Clause 109, wherein L⁵⁰ is a substituted 2 to 10 membered heteroalkylene.

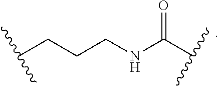 [SMILES: *CCCNC(*)=O]

Clause 141: The method of Clause 109, wherein L⁵⁰ is H
Clause 142: The method of Clause 109, wherein R⁵⁰ is a fluorescent moiety.
Clause 143: The method of Clause 142, wherein R⁵⁰ is a monovalent form of FAM, a monovalent form of VIC, a monovalent form of ABY, a monovalent form of JUN, a monovalent form of AF647, a monovalent form of Cy5, a monovalent form of AF676, or a monovalent form of Cy5.5.
Clause 144: The method of Clause 109, wherein R² is hydrogen or —OH.
Clause 145: The method of Clause 109, wherein R² is hydrogen.
Clause 146: The method of any one of Clauses 109 to 145, wherein R³⁰ is —OH.
Clause 147: The method of any one of Clauses 109 to 145, wherein R³⁰ is

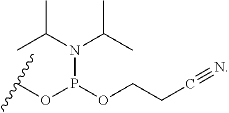 [SMILES: *OP(OCCC#N)N(C(C)C)C(C)C]

Clause 148: The method of any one of Clauses 109 to 145, wherein the 3′ blocking moiety is a monovalent form of dideoxycytidine (3′ddC), a monovalent form of dideoxyadenosine (ddA), 3′ Inverted dT, 3′ amino modifier, a monovalent form of QSY7, a monovalent form of QSY21, a monovalent form of QSY9, a monovalent form of BHQ1, a monovalent form of BHQ2, a monovalent form of BHQ3, a monovalent form of Dabcyl, a monovalent form of Dabsyl, a monovalent form of Eclipse, a monovalent form of BBQ-650, a monovalent form of Iowa Black RQ, a monovalent form of Iowa Black FQ,

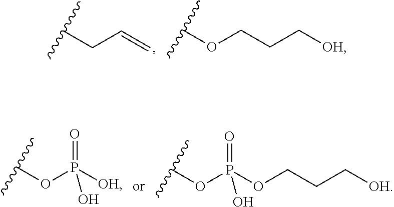

Clause 149: A composition for detecting nucleic acids in a sample, the composition comprising:
a first probe detectably labeled with a first label configured to generate a first emission signal,
a second probe detectably labeled with a second label configured to generate a second emission signal,
wherein the first and second probes have different sequences, and
wherein the first and second labels are identical and/or generate substantially identical emission
wherein under a first set of conditions, the first label generates a first emission signal that increases as a result of specific interaction of the first probe with a first nucleic acid target, and second label a second emission signal that is not increased as a result of specific interaction of the second probe with a second nucleic acid target; and
wherein under a second set of conditions different from the first set of conditions, the second emission signal is increases as a result of specific interaction of the second probe with the second nucleic acid target.
Clause 150: The composition according to Clause 149, wherein the first probe is a cleavable probe.
Clause 151: The composition according to any one of Clauses 149 and 150, wherein the first probe includes a fluorophore and a quencher, and wherein the first probe is configured such that fluorescence from the fluorophore is quenched by the quencher until the probe is cleaved during an annealing/extension stage of the amplification process.
Clause 152: The composition according to any one of Clauses 149-151, wherein the first probe is a TaqMan probe.
Clause 153: The composition according to any one of Clauses 149-152, wherein the second probe is a non-cleavable probe.
Clause 154: The composition according to any one of Clauses 149-152, wherein the second probe comprises a stem-loop portion configured to form a stem-loop structure when the second probe is single-stranded.
Clause 155: The composition according to any one of Clauses 153 or 154, wherein the second probe comprises a fluorophore and a quencher spaced apart from one another such that the fluorophore is quenched when the second probe is single-stranded and unquenched when the second probe is incorporated into a double-stranded amplicon.
Clause 156: The composition according to Clause 155, wherein the fluorophore is located at or near the 5′ end of the second probe and the quencher is 3′ of the fluorophore.
Clause 157: The composition of any one of Clauses 155 or 156, wherein both the fluorophore and the quencher are disposed at or near the stem loop portion of the second probe.
Clause 158: The composition of any one of Clauses 149-157, wherein the reaction mixture further comprises: a first primer pair complementary to a first nucleic acid target of the nucleic acids or its complement, the first nucleic acid target being configured to generate a first amplicon with which the first probe can hybridize; and a second primer pair complementary to a second nucleic acid target of the nucleic acids or its complement, the second nucleic acid target being configured to generate a second amplicon with which the second probe can hybridize.
Clause 159: The composition of Clause 158, wherein the second primer pair includes a primer with a tail.
Clause 160: The composition of Clause 159, wherein the tail forms the 5′ end of the primer with the tail.
Clause 161: The composition of any one of Clauses 159 or 160, wherein the second probe can hybridize to the tail or to its complement.
Clause 162: The composition of any one of Clauses 159-161, wherein the second primer pair further includes a non-tailed primer, and wherein a concentration of the primer with the tail in the reaction mixture is different from that of the non-tailed primer in the reaction mixture.
Clause 163: The composition of Clause 162, wherein the concentration of the non-tailed primer is greater than that of the primer with the tail.
Clause 164: The composition of Clause 163, wherein the concentration of the non-tailed primer is about 2× to about 30× greater than the concentration of the primer with the tail, or about 5× to about 25× greater than the concentration of the primer with the tail, or about 10× to about 20× greater than the concentration of the primer with the tail.
Clause 165: The composition of any one of Clauses 162-164, wherein the second probe is provided at a concentration that is different from the concentration of the primer with the tail and the concentration of the non-tailed primer.
Clause 166: The composition of Clause 165, wherein the second probe is provided at a concentration that is greater than the concentration of the primer with the tail.
Clause 167: The composition of any one of Clauses 165 or 166, wherein the second probe is provided at a concentration that is less than the concentration of the non-tailed primer.
Clause 168: The composition of any one of Clauses 165-167, wherein the second probe is provided at a concentration that is about 2× to about 10× the concentration of the primer with the tail, or about 3× to about 7.5× the concentration of the primer with the tail.
Clause 169: A composition, comprising:
a primer pair complementary to a nucleic acid target or its complement for generating an amplicon, and
a non-cleavable probe configured to hybridize to the amplicon, the non-cleavable probe including a detectable label configured to provide an emission signal that corresponds to an amount of generated amplicon,
wherein the detectable label generates emission without cleavage of the non-cleavable probe during an amplification process including a series of thermal cycling stages that includes at least two different target temperatures.
Clause 170: The composition of Clause 169, wherein the composition is a reaction mixture.
Clause 171: The composition of Clauses 169 or 170, wherein the non-cleavable probe comprises a stem-loop portion capable of forming a stem-loop structure when the non-cleavable probe is single-stranded.
Clause 172: The composition of any one of Clauses 159-170, wherein the non-cleavable probe comprises a fluorophore and a quencher spaced such that the fluorophore is quenched when the non-cleavable probe is single-stranded but enabled when the probe is incorporated into a double-stranded amplicon.
Clause 173: The composition of Clause 172, wherein the fluorophore is located at or near the 5′ end of the probe and the quencher is 3′ of the fluorophore.
Clause 174: The composition of Clause 172, wherein both the fluorophore and the quencher are at or near the stem loop portion of the probe.
Clause 175: The composition of any one of Clauses 169-174, wherein the primer pair includes a primer with a tail.
Clause 176: The composition of Clause 175, wherein the tail forms the 5′ end of the primer with the tail.
Clause 177: The composition of Clause 175 or Clause 176, wherein the non-cleavable probe is configured to hybridize to the tail or to its complement.
Clause 178: The composition of Clause 177, wherein a 3′ portion of the non-cleavable probe is configured to hybridize to the tail or its complement.
Clause 179: The composition of any one of Clauses 169, wherein the primer pair includes a primer with a tail and a non-tailed primer provided at different concentrations.
Clause 180: The composition of Clause 179, wherein the non-tailed primer is provided at a greater concentration than the primer with the tail.
Clause 181: The composition of Clause 180, wherein the non-tailed primer is provided at a concentration that is about 2× to about 30× the concentration of the primer with the tail, or about 5× to about 25× the concentration of the primer with the tail, or about 10× to about 20× the concentration of the primer with the tail.
Clause 182: The composition of Clause 179, wherein a concentration of the non-cleavable probe in the reaction mixture is greater than a concentration of the primer with the tail in the reaction mixture.
Clause 183: The composition of Clause 179, wherein the non-cleavable probe is provided at a concentration that is less than the concentration of the non-tailed primer.
Clause 184: The composition of any one of Clause 183, wherein the non-cleavable probe is provided at a concentration that is about 2× to about 10× the concentration of the non-tailed primer, or about 3× to about 7.5× the concentration of the non-tailed primer.
Clause 185: The composition of any one of Clauses 169-184,
wherein the reaction mixture further comprises:
   a primer pair complementary to a second nucleic acid target or its complement for generating a second amplicon, and
   a cleavable probe configured to hybridize to the second amplicon, the cleavable probe including a detectable label configured to provide an emission signal that corresponds to an amount of generated second amplicon;
wherein subjecting the reaction mixture to the amplification process generates second amplicons, wherein the detectable label of the cleavable probe generates emission due to cleavage of the cleavable probe during the amplification process; and
wherein the method further comprises measuring the emission signal from the cleavable probe.
Clause 186: A kit comprising, the composition of any one of Clauses 149-185.

Those having ordinary skill in the art would understand that various other modifications to structure, arrangements, methods, materials and the like may be made without departing from the scope of the present disclosure and principles of operation. By way of example, while various embodiments describe methods for utilizing a first probe and a second probe having spectral similarity to perform detection of two different targets, the methods could involve other numbers of targets and probes to increase the plexy of an overall amplification and detection assay.

Those of ordinary skill in the art would appreciate how any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures.

The described embodiments are to be considered in all respects only as illustrative and not restrictive. All changes which come within the meaning and range of equivalency of the claims are to be embraced within their scope.

1. A method of detecting nucleic acids in a sample, comprising:
(i) providing a reaction mixture, the reaction mixture including
at least a portion of the sample,
a first probe detectably labeled with a first label configured to generate a first emission signal,
a second probe detectably labeled with a second label configured to generate a second emission signal,
wherein the first and second probes have different sequences, and
wherein the first and second labels are identical and/or generate substantially identical emission;
(ii) subjecting the reaction mixture to an amplification process comprising a first set of reaction conditions and a second set of reaction conditions, the first set of reaction conditions being different than the second set of reaction conditions; and
(iii) determining the presence of the first and/or second nucleic acid targets in the sample by
measuring an emission signal during the first set of reaction conditions, the emission signal during the first set of reaction conditions being correlated with specific interaction or lack of interaction between the first probe and a first nucleic acid target,
measuring an emission signal during the second set of reaction conditions, the emission signal during the second set of reaction conditions being correlated with specific interaction or lack of interaction between the first probe and a first nucleic acid target and with specific or lack of interaction between the second probe and a second nucleic acid target, and
estimating the presence and/or amount of each of the first nucleic acid target and second nucleic acid target.

2. A method of detecting nucleic acids in a sample, comprising:
(i) providing a reaction mixture, the reaction mixture including:
at least a portion of the sample,
a first probe detectably labeled with a first label configured to generate a first emission signal that is indicative of the presence or absence of a first nucleic acid target,
a second probe detectably labeled with a second label configured to generate a second emission signal that is indicative of the presence or absence of a second nucleic acid target,
wherein the first and second probes have different sequences, and
wherein the first and second labels are identical and/or generate substantially identical emission signals;
(ii) subjecting the reaction mixture to an amplification process comprising a first set of reaction conditions and a second set of reaction conditions, the first set of reaction conditions being different than the second set of reaction conditions; and
(iii) determining any presence and/or amount of each of the first and/or second nucleic acid targets in the reaction mixture by
measuring during the first set of reaction conditions a first total emission signal that comprises any first emission signal if present and any second emission signal if present,
measuring during the second set of reaction conditions a second total emission signal comprising any first emission signal if present and any second emission signal if present, and
estimating the first emission signal and/or second emission signal based on the first and second total emission signals.

3. The method of claim 1 or 2, wherein the first and second emission signals are first and second fluorescence signals, and wherein both the first and second probes are subjected to excitation at the same wavelength and/or both the first and second probes are subjected to excitation during detection of their respective first and second fluorescence signals.

4. The method of any one of claims 1-3, wherein the emission signals are fluorescence signals and wherein:
measuring the fluorescence signal during the first set of reaction conditions comprises measuring a combined signal comprising the first and second fluorescence signals during the first set of reaction conditions to obtain a first total fluorescence signal;
measuring the fluorescence signal during the second set of reaction conditions comprises measuring a combined signal comprising the first and second fluorescence signals during the second set of reaction conditions to obtain a second total fluorescence signal; and
estimating the presence and/or amount of each of the first nucleic acid target and the second nucleic acid target comprises estimating the first fluorescence signal and/or the second fluorescence signal based on the first and second total fluorescence signals.

5. The method of claim 4, wherein the second fluorescence signal differs between the first and second set of reaction conditions to a greater degree than the first fluorescence signal differs between the first and second set of reaction conditions.

6. The method of any one of claims 4-5, wherein
the first total fluorescence value comprises (i) fluorescence from first label that is free and unquenched within the reaction mixture and emitted as a result of the first label being cleaved following hybridization of the first probe to the first amplicon, and (ii) background fluorescence of the second label, and
the second total fluorescence value is based on (i) fluorescence from first label that is free and unquenched within the reaction mixture and emitted as a result of the first label being cleaved following hybridization of the first probe to the first amplicon, and (ii) fluorescence from the second label, above the background fluorescence of the second label, emitted as a result of hybridization of the second probe to the second amplicon.

7. The method of any one of claims 4-6, further comprising:
calculating an amount of the first nucleic acid target based on the first fluorescent signal; and
calculating an amount of the second nucleic acid target based on the second fluorescent signal.

8. The method of any one of claims 4-7, wherein the first fluorescent signal being above a background level during both the first and second sets of reaction conditions indicates presence of the first nucleic acid target in the reaction mixture.

9. The method of any one of claims 4-8, wherein the second fluorescent signal being above a background level during the second set of reaction conditions but not during the first set of reaction conditions indicates presence of the second nucleic acid target in the reaction mixture.

10. The method of any one of claims 5-8, wherein the first set of reaction conditions comprises a first measurement temperature at which the first fluorescence signal is measured, and the second set of reaction conditions comprises a second measurement temperature at which the second fluorescence signal is measured, the second measurement temperature being different than the first measurement temperature.

11. The method of claim 10, wherein the first and second measurement temperatures differ by at least about 10° C. or more, about 15° C. or more, about 20° C. or more, about 25° C. or more, or about 30° C. or more.

12. The method of claim 10 or claim 11, wherein at least one of the first or second measurement temperatures is a denaturation temperature at which DNA in the reaction mixture is denatured, such as in a range of about 80° C. or above.

13. The method of any one of claims 1-12, wherein the reaction mixture is subjected to multiple amplification cycles during the amplification process, each of the amplification cycles comprising the first and second set of reaction conditions.

14. The method of any one of claims 1-13, wherein the amplification process comprises thermal cycling.

15. The method of claim 14, wherein the subjecting the reaction mixture to the first set of reaction conditions comprises thermal cycling the reaction mixture at a first temperature sufficient to cause denaturation of the first and second amplicons.

16. The method of claim 15, wherein the subjecting the reaction mixture to the second set of reaction conditions comprises thermal cycling the reaction mixture at a second temperature sufficient to cause annealing and/or extension of the first nucleic acid target and the second nucleic acid target to respectively form the first amplicon and the second amplicon, the second temperature being lower than the first temperature.

17. The method of any one of claims 1-16 wherein the first probe is a cleavable probe.

18. The method of claim 17, wherein the first emission signal increases as the cleavable probe is cleaved during an annealing/extension stage.

19. The method of claim 17 or claim 18, wherein the first probe includes a fluorophore and a quencher, and wherein the first probe is configured such that fluorescence from the fluorophore is quenched by the quencher until the probe is cleaved during an annealing/extension stage of the amplification process.

20. The method of claim 19, wherein the first probe is a TaqMan probe.

21. The method of any one of claims 1-20, wherein the second probe is a non-cleavable probe.

22. The method of claim 21, wherein the second probe comprises a stem-loop portion configured to form a stem-loop structure when the second probe is single-stranded.

23. The method of claim 21 or claim 22, wherein the second probe comprises a fluorophore and a quencher spaced apart from one another such that the fluorophore is quenched when the second probe is single-stranded and unquenched when the second probe is incorporated into a double-stranded amplicon.

24. The method of claim 23, wherein the fluorophore is located at or near the 5′ end of the second probe and the quencher is 3′ of the fluorophore.

25. The method of claim 23 or claim 24, wherein both the fluorophore and the quencher are disposed at or near the stem loop portion of the second probe.

26. The method of any one of claims 1-25, wherein the reaction mixture further comprises: a first primer pair complementary to a first nucleic acid target of the nucleic acids or its complement, the first nucleic acid target being configured to generate a first amplicon with which the first probe can hybridize; and a second primer pair complementary to a second nucleic acid target of the nucleic acids or its complement, the second nucleic acid target being configured to generate a second amplicon with which the second probe can hybridize.

27. The method of claim 26, wherein the second primer pair includes a primer with a tail.

28. The method of claim 27, wherein the tail forms the 5′ end of the primer with the tail.

29. The method of claim 27 or claim 28, wherein the second probe can hybridize to the tail or to its complement.

30. The method of any one of claims 20-29, wherein the amplification process utilizes a series of thermal cycling stages that includes at least three different target temperatures.

31. The method of claim 30, wherein the amplification process includes a denaturation temperature and multiple different annealing/extension temperatures that vary throughout the amplification process.

32. The method of claim 31, wherein a first series of denaturation and annealing/extension stages are carried out at a first annealing/extension temperature, and wherein a second series of denaturation and annealing/extension stages are carried out at a second annealing/extension temperature different from the first annealing/extension temperature.

33. The method of claim 32, wherein the first annealing/extension temperature is higher than the second annealing/extension temperature.

34. The method of claim 32 or claim 33, wherein the first series of denaturation and annealing/extension stages are cycled a greater number of times than the second series of denaturation and annealing/extension stages.

35. The method of any one of claims 32-34, wherein the amplification process further comprises a third series of denaturation and annealing/extension steps carried out using a third annealing/extension temperature.

36. The method of claim 35, wherein the third annealing/extension temperature is the same as the first annealing/extension temperature.

37. The method of claim 35 or claim 36, wherein the third series of denaturation and annealing/extension stages are cycled a greater number of times than the first series of denaturation and annealing/extension stages.

38. The method of any one of claims 32-37, wherein the denaturation temperature is the same for each series of the denaturation stages.

39. The method of any one of claims 27-38, wherein the second primer pair further includes a non-tailed primer, and wherein a concentration of the primer with the tail in the reaction mixture is different from that of the non-tailed primer in the reaction mixture.

40. The method of claim 39, wherein the concentration of the non-tailed primer is greater than that of the primer with the tail.

41. The method of claim 40, wherein the concentration of the non-tailed primer is about 2× to about 30× greater than the concentration of the primer with the tail, or about 5× to about 25× greater than the concentration of the primer with the tail, or about 10× to about 20× greater than the concentration of the primer with the tail.

42. The method of any one of claims 39-41, wherein the second probe is provided at a concentration that is different from the concentration of the primer with the tail and the concentration of the non-tailed primer.

43. The method of claim 42, wherein the second probe is provided at a concentration that is greater than the concentration of the primer with the tail.

44. The method of claim 42 or claim 43, wherein the second probe is provided at a concentration that is less than the concentration of the non-tailed primer.

45. The method of any one of claims 42-44, wherein the second probe is provided at a concentration that is about 2× to about 10× the concentration of the primer with the tail, or about 3× to about 7.5× the concentration of the primer with the tail.

46. The method of any one of claims 1-45, wherein a melting temperature (Tₘ) of the first probe and a Tₘ of the second probe are within about 8° C., or about 6° C., or about 4° C., or about 2° C. of each other.

47. The method of any one of claims 1-48, wherein the amplification process cycles between at least two target temperatures for multiple cycles of the amplification process.

48. The method of claim 47, wherein the amplification process cycles between at least two target temperatures for at least 5% of, at least 10% of, at least 15% of, at least 20% of, at least 25% of, at least 30% of, at least 35% of, at least 40%, of, at least 45% of, at least 50% of, at least 55% of, at least 60% of, at least 65% of, at least 70% of, at least 75% of, at least 80% of, at least 85% of, at least 90% of, or at least 95% of the cycles of the amplification process.

49. The method of any one of claims 1-48, wherein the method further comprises partitioning the reaction mixture into a plurality of reaction volumes, and wherein the amplification process is a digital PCR (dPCR) process.

50. The method of claim 49, wherein measuring the emission signal during the first set of reaction conditions comprises measuring the emission signal upon or near completion of the subjecting the reaction mixture to the first set of reaction conditions to obtain a first end-point measurement, and wherein measuring the emission signal during the second set of reaction conditions comprises measuring the emission signal upon or near completion of the subjecting the reaction mixture to the second set of reaction conditions to obtain a second end-point measurement.

51. The method of claim 50, wherein the estimating the presence and/or amount of each of the first nucleic acid target and the second nucleic acid target comprises:
categorizing the plurality of reaction volumes according to the emission signal measured at the first end-point measurement and according to the emission signal measured at the second end-point measurement; and
based on the categorizations, determining a count for the plurality of reaction volumes in which the first probe showed activity and a count for the plurality of reaction volumes in which the second probe showed activity.

52. The method of any one of claims 1-48, wherein the measuring the emission signal during the first set of reaction conditions comprises measuring the emission signal during a denaturation stage of an end-point cycle of the amplification process, and wherein the measuring the emission signal during the second set of reaction conditions comprises measuring the emission signal during an annealing and/or extension state of the end-point cycle of the amplification process.

53. The method of any one of claims 1-48 or 52, wherein the amplification process is an end-point PCR process.

54. A method of detecting nucleic acids in a sample, comprising:
providing a reaction mixture, the reaction mixture comprising:
a primer pair complementary to a nucleic acid target or its complement for generating an amplicon, and
a non-cleavable probe configured to hybridize to the amplicon, the non-cleavable probe including a detectable label configured to provide an emission signal that corresponds to an amount of generated amplicon,
subjecting the reaction mixture to an amplification process to generate the amplicons, wherein the label generates emission without cleavage of the non-cleavable probe during the amplification process, and wherein the amplification process utilizes a series of thermal cycling stages that includes at least three different target temperatures, and
measuring the emission signal from the non-cleavable probe.

55. The method of claim 54, further comprising quantitating an amount of the nucleic acid target based on the measured emission signal.

56. The method of claim 54 or claim 55, wherein the non-cleavable probe comprises a stem-loop portion capable of forming a stem-loop structure when the non-cleavable probe is single-stranded.

57. The method of any one of claims 54-56, wherein the non-cleavable probe comprises a fluorophore and a quencher spaced such that the fluorophore is quenched when the non-cleavable probe is single-stranded but enabled when the probe is incorporated into a double-stranded amplicon.

58. The method of claim 57, wherein the fluorophore is located at or near the 5′ end of the probe and the quencher is 3′ of the fluorophore.

59. The method of claim 57 or claim 58, wherein both the fluorophore and the quencher are at or near the stem loop portion of the probe.

60. The method of any one of claims 54-59, wherein the primer pair includes a primer with a tail.

61. The method of claim 60, wherein the tail forms the 5′ end of the primer with the tail.

62. The method of claim 60 or claim 61, wherein the non-cleavable probe is configured to hybridize to the tail or to its complement.

63. The method of claim 62, wherein a 3′ portion of the non-cleavable probe is configured to hybridize to the tail or its complement.

64. The method of any one of claims 54-63, wherein the amplification process includes a denaturation temperature and multiple different annealing/extension temperatures that vary throughout the amplification process.

65. The method of claim 64, wherein a first series of denaturation and annealing/extension stages are carried out at a first annealing/extension temperature, and wherein a second series of denaturation and annealing/extension stages are carried out at a second annealing/extension temperature that is different from the first annealing/extension temperature.

66. The method of claim 65, wherein the first annealing/extension temperature is higher than the second annealing/extension temperature.

67. The method of claim 65 or claim 66, wherein the first series of denaturation and annealing/extension stages are cycled a greater number of times than the second series of denaturation and annealing/extension stages.

68. The method of any one of claims 65-67, wherein the amplification process further comprises a third series of denaturation and annealing/extension stages carried out using a third annealing/extension temperature.

69. The method of claim 68, wherein the third annealing/extension temperature is the same as the first annealing/extension temperature.

70. The method of claim 68 or claim 69, wherein the third series of denaturation and annealing/extension stages are cycled a greater number of times than the first series of denaturation and annealing/extension stages.

71. The method of any one of claims 65-70, wherein the denaturation temperature is the same for each series of denaturation stages.

72. The method of any one of claims 65-71, wherein the primer pair further includes a non-tailed primer, and wherein a concentration of the primer with the tail in the reaction mixture is different than that of the non-tailed primer.

73. The method of claim 72, wherein the non-tailed primer is provided at a greater concentration than the primer with the tail.

74. The method of claim 73, wherein the non-tailed primer is provided at a concentration that is about 2× to about 30× the concentration of the primer with the tail, or about 5× to about 25× the concentration of the primer with the tail, or about 10× to about 20× the concentration of the primer with the tail.

75. The method of any one of claims 74-74, wherein a concentration of the non-cleavable probe in the reaction mixture is different than a concentration of the primer with the tail and a concentration of the non-tailed primer in the reaction mixture.

76. The method of claim 75, wherein a concentration of the non-cleavable probe in the reaction mixture is greater than a concentration of the primer with the tail in the reaction mixture.

77. The method of claim 75 or claim 76, wherein the non-cleavable probe is provided at a concentration that is less than the concentration of the non-tailed primer.

78. The method of any one of claims 77-77, wherein the non-cleavable probe is provided at a concentration that is about 2× to about 10× the concentration of the non-tailed primer, or about 3× to about 7.5× the concentration of the non-tailed primer.

79. The method of any one of claim 54 or 55,
wherein the reaction mixture further comprises:
a primer pair complementary to a second nucleic acid target or its complement for generating a second amplicon, and
a cleavable probe configured to hybridize to the second amplicon, the cleavable probe including a detectable label configured to provide an emission signal that corresponds to an amount of generated second amplicon;
wherein the subjecting the reaction mixture to the amplification process generates second amplicons, wherein the detectable label of the cleavable probe generates emission due to cleavage of the cleavable probe during the amplification process; and
wherein the method further comprises measuring the emission signal from the cleavable probe.

80. The method of claim 81, further comprising quantitating an amount of the second nucleic acid target based on the measured emission signals.

81. A method of detecting nucleic acids in a sample, comprising:
providing a reaction mixture, the reaction mixture comprising:
a primer pair targeted to a nucleic acid target for generating an amplicon, the primer pair including a primer with the tail and a non-tailed primer provided at different concentrations, and
a detectably labelled, non-cleavable probe configured to hybridize to the amplicon and to generate a fluorescent signal that corresponds to an amount of generated amplicon,
subjecting the reaction mixture to an amplification process to generate the amplicons, wherein the non-cleavable probe generates emission without being cleaved during the amplification process; and
measuring the emission signal from the non-cleavable probe.

82. The method of claim 81 wherein the non-tailed primer is provided at a greater concentration than the primer with the tail.

83. The method of claim 82, wherein the non-tailed primer is provided at a concentration that is about 2× to about 30× the concentration of the primer with the tail, or about 5× to about 25× the concentration of the primer with the tail, or about 10× to about 20× the concentration of the primer with the tail.

84. The method of any one of claims 80-83, wherein the non-cleavable probe is provided at a concentration that is different from the concentration of the primer with the tail and the concentration of the non-tailed primer.

85. The method of claim 84, wherein the non-cleavable probe is provided at a concentration that is greater than the concentration of the primer with the tail.

86. The method of claim 84 or claim 84, wherein the non-cleavable probe is provided at a concentration that is less than the concentration of the non-tailed primer.

87. The method of any one of claims 84-86, wherein the non-cleavable probe is provided at a concentration that is about 2× to about 10× the concentration of the non-tailed primer, or about 3× to about 7.5× the concentration of the non-tailed primer.

88. The method of claim 81,
wherein the reaction mixture further comprises:
a primer pair targeted to a second nucleic acid target different from the nucleic acid target and for generating a second amplicon, the primer pair including a primer with the tail and a non-tailed primer provided at different concentrations, and
a detectably labelled, cleavable probe configured to hybridize to the second amplicon and to generate an emission signal that corresponds to an amount of generated second amplicon;
wherein subjecting the reaction mixture to an amplification process generates the second amplicons, wherein the cleavable probe generates emission signal without due to cleavage during the amplification process; and
wherein the method further comprises measuring the emission signal from the cleavable probe.

89. A method of detecting the presence or amount of a first and/or second target in a reaction mixture, comprising:
including a first and second probe in the reaction mixture, wherein the first probe can specifically interact with a first target and comprises a first label that can produce a first detectable signal, and the second probe can specifically interact with a second target and comprises a second label that can produce a second detectable signal;
allowing specific interaction of the first and second probe with any first and second target, respectively, in the reaction mixture;
measuring a first total signal through an optical filter under a first set of conditions, wherein the first total signal includes the first and second detectable signals from the first and second labels, and wherein under the first set of conditions, the first detectable signal is increased as a result of specific interaction of the first probe with the first target, but the second detectable signal is not increased as a result of specific interaction of the second probe with the second target;
measuring a second total signal through the same optical filter under a second set of conditions, wherein the second total signal includes the first and second detectable signals from the first and second labels, and wherein under the second set of conditions, the second detectable signal is increased as a result of specific interaction of the second probe with the second target; and
assessing the presence or amount of the first and/or second target, by estimating the first detectable signal and the second detectable signal based on both the first total signal and the second total signal.

90. The method of claim 89, wherein the first and second labels are identical and/or generate substantially identical fluorescence.

91. The method of claim 89 or claim 90, wherein the second fluorescence signal differs between the first and second set of conditions to a greater degree than the first fluorescence signal differs between the first and second set of conditions.

92. The method of any one of claims 89-91, wherein the first probe is a cleavable probe.

93. The method of claim 92, wherein the first detectable signal increasing indicates the cleavable probe is cleaved.

94. The method of claim 92 or claim 93, wherein the first probe includes a fluorophore and a quencher, and wherein the first probe is configured such that fluorescence from the fluorophore is quenched by the quencher until the probe is cleaved.

95. The method of claim 94, wherein the first probe is a TaqMan probe.

96. The method of any one of claims 89-95, wherein the second probe is a non-cleavable probe.

97. The method of claim 96, wherein the second probe comprises a stem-loop portion capable of forming a stem-loop structure when the second probe is single-stranded.

98. The method of claim 96 or claim 97, wherein the second label of the second probe is a fluorophore, wherein the second probe further comprises a quencher spaced such that the fluorophore is quenched when the second probe is single-stranded but enabled when the second probe is incorporated into a double-stranded nucleic acid.

99. The method of claim 98, wherein the fluorophore is located at or near the 5′ end of the second probe and the quencher is 3′ of the fluorophore.

100. The method of claim 98 or claim 99, wherein both the fluorophore and the quencher are disposed at or near the stem loop portion of the second probe.

101. The method of any one of claims 89-100, wherein a melting temperature (Tₘ) of the first probe and a Tₘ of the second probe are within about 8° C., or about 6° C., or about 4° C., or about 2° C. of each other.

102. The method of any one of claims 89-101, wherein the first set of conditions comprises a first measurement temperature at which the first fluorescence signal is measured, and the second set of conditions comprises a second, different measurement temperature at which the second fluorescence signal is measured.

103. The method of claim 102, wherein the first and second measurement temperatures differ by at least about 10° C. or more, about 15° C. or more, about 20° C. or more, about 25° C. or more, or about 30° C. or more.

104. The method of claim 102 or claim 103, wherein at least one of the first or second measurement temperatures is a denaturation temperature at which DNA in the reaction mixture is denatured, such as about 90° C. or above.

105. The method of any one of claims 89-104, further comprising thermal cycling of the reaction mixture between two target temperatures for multiple cycles.

106. The method of claim 105, wherein the thermal cycling cycles between two target temperatures for at least 5% of, at least 10% of, at least 15% of, at least 20% of, at least 25% of, at least 30% of, at least 35% of, at least 40%, of, at least 45% of, at least 50% of, at least 55% of, at least 60% of, at least 65% of, at least 70% of, at least 75% of, at least 80% of, at least 85% of, at least 90% of, or at least 95% of the cycles.

107. The method of any one of claims 54-106, wherein measuring the signals occurs at an end-point thermal cycle of the amplification process.

108. The method of any one of claims 1, 2, or 86, wherein the first probe is configured to produce a cumulative signal across differing stages of a cycle of an amplification process and the second probe is configured to produce a transient signal during differing stages of a cycle of an amplification process.

109. The method of any one of claims 1-108, wherein the second probe is a compound having the formula: 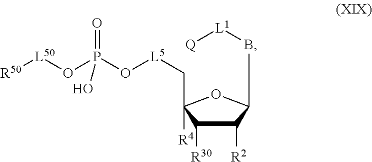
or a salt thereof, wherein
Q is an internal quencher moiety having the formula: 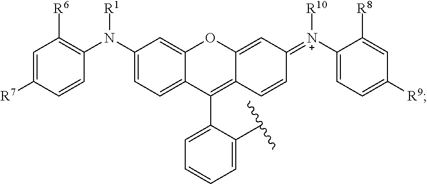
B is a divalent nucleobase;
Li is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is the second label or the detectable label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, □NHNH₂, □ONH₂, □NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R¹ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂L, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

110. The method of claim 109, wherein L⁵ forms a stem-loop structure when the second probe is single-stranded.

111. The method of claim 109, wherein R⁵⁰ is a fluorophore, and Q and R⁵⁰ are spaced apart from one another such that R⁵⁰ is quenched when the second probe is single stranded and unquenched when the second probe is incorporated into a double-stranded amplicon.

112. The method of claim Error! Reference source not found., wherein both Q and R⁵⁰ are disposed at or near the stem loop portion of the second probe.

113. The method of claim 109, wherein L⁵ comprises from 11 to 30 nucleotides.

114. The method of claim 109, wherein L⁵ comprises from 19 to 23 nucleotides.

115. The method of claim 109, wherein L⁵ comprises from 4 to 14 nucleotides.

116. The method of claim 109, wherein L⁵ comprises from 6 to 12 nucleotides.

117. The method of claim 109, wherein the nucleotides are DNA nucleotides.

118. The method of claim 109, wherein the nucleotides are RNA molecules.

119. The method of claim 109, wherein the compound has the formula:

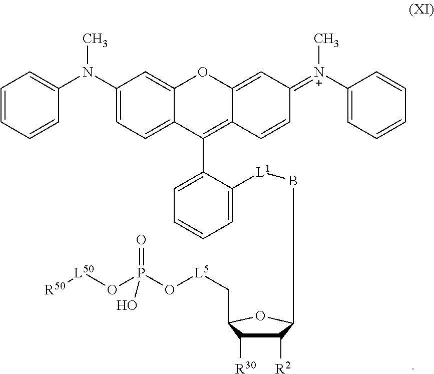

120. The method of claim 109, wherein the compound has the formula:

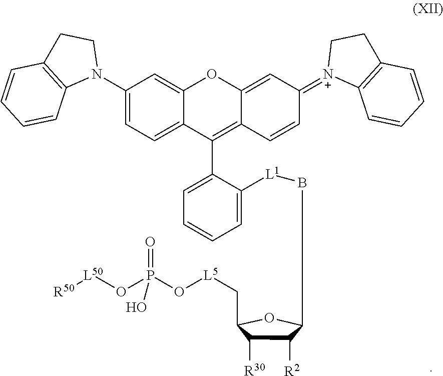

121. The method of claim 109, wherein the compound has the formula:

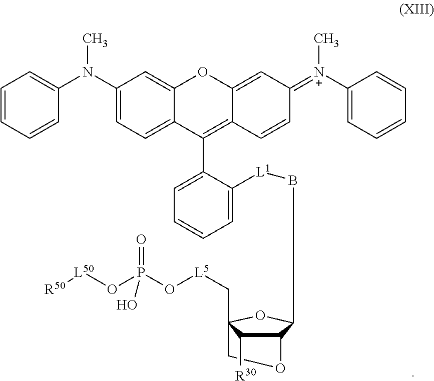

122. The method of claim 109, wherein the compound has the formula:

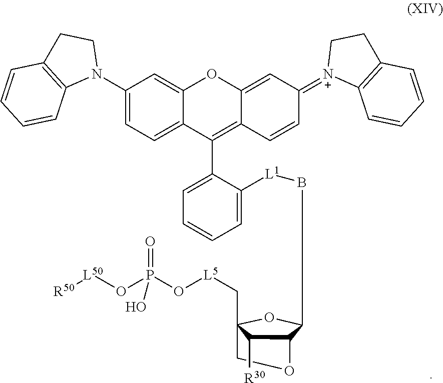

123. The method of claim 109, wherein B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof.

124. The method of claim 109, wherein B is a divalent cytosine or a derivative thereof, divalent guanine or a derivative thereof, divalent adenine or a derivative thereof, divalent thymine or a derivative thereof, or divalent uracil or a derivative thereof.

125. The method of claim 109, wherein the compound has the formula:

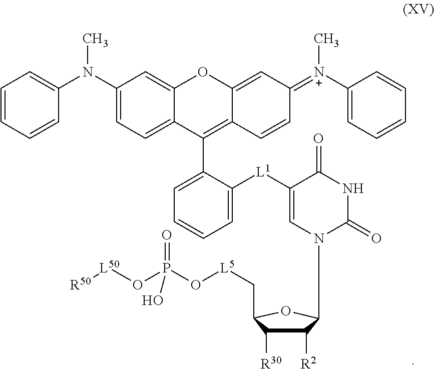

126. The method of claim 109, wherein the compound has the formula:

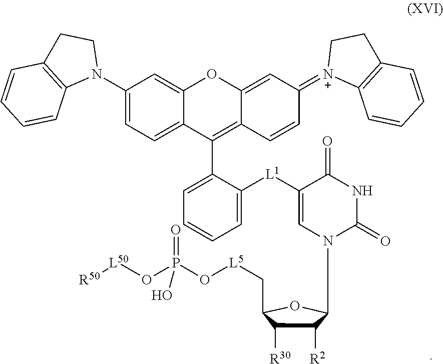

127. The method of claim 109, wherein the compound has the formula:

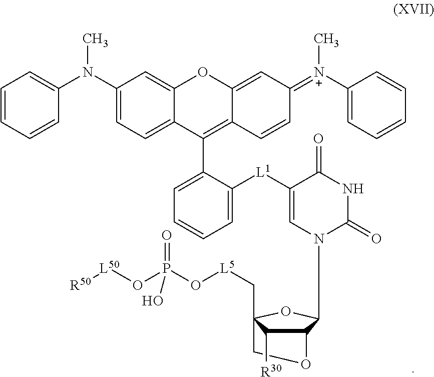

128. The method of claim 109, wherein the compound has the formula:

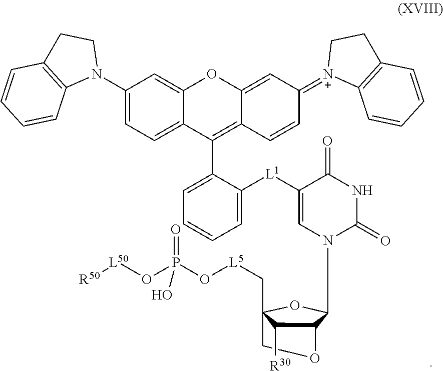

129. The method of claim 109, wherein L¹ is L¹⁰¹-L¹⁰²-L¹⁰³-L¹⁰⁴-L¹⁰⁵; and
L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

130. The method of claim 129, wherein L¹⁰¹ is —S(O)₂—.

131. The method of claim 129, wherein L¹⁰² is an unsubstituted 3 to 8 membered heterocycloalkyl.

132. The method of claim 129, wherein L¹⁰² is an unsubstituted piperidinyl.

133. The method of claim 129, wherein L¹⁰² is

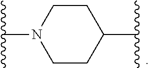 [SMILES: *C1CCN(*)CC1]

134. The method of claim 129, wherein L¹⁰³ is —C(O)NH—.

135. The method of claim 129, wherein L¹⁰⁴ is an unsubstituted C₁-C₁₀ alkylene, unsubstituted 2 to 6 membered heteroalkylene, or unsubstituted phenylene.

136. The method of claim 129, wherein L¹⁰⁴ is an unsubstituted n-hexylene,

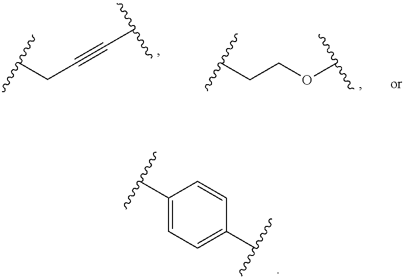 [SMILES: *C#CC* ; *CCO* ; *c1ccc(*)cc1]

137. The method of claim 129, wherein L¹⁰⁵ is an unsubstituted C₁-C₁₀ alkylene, substituted or unsubstituted 2 to 8 membered heteroalkylene, or unsubstituted 5 to 10 membered heteroarylene.

138. The method of claim 129, wherein L¹⁰⁵ is

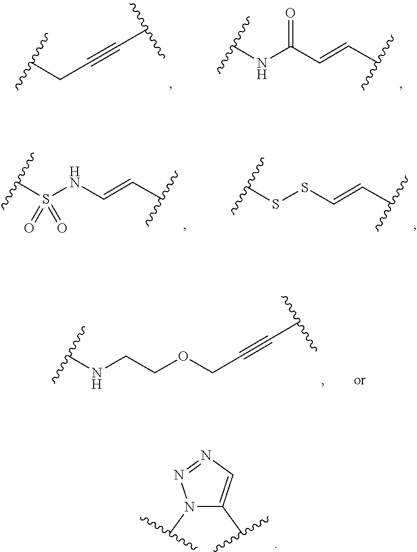 [SMILES: *C#CC* ; */C=C/C(=O)N* ; */C=C/NS(*)(=O)=O ; */C=C/SS* ; *C#CCOCCN* ; *c1cnnn1*]

139. The method of claim 129, wherein L¹ is

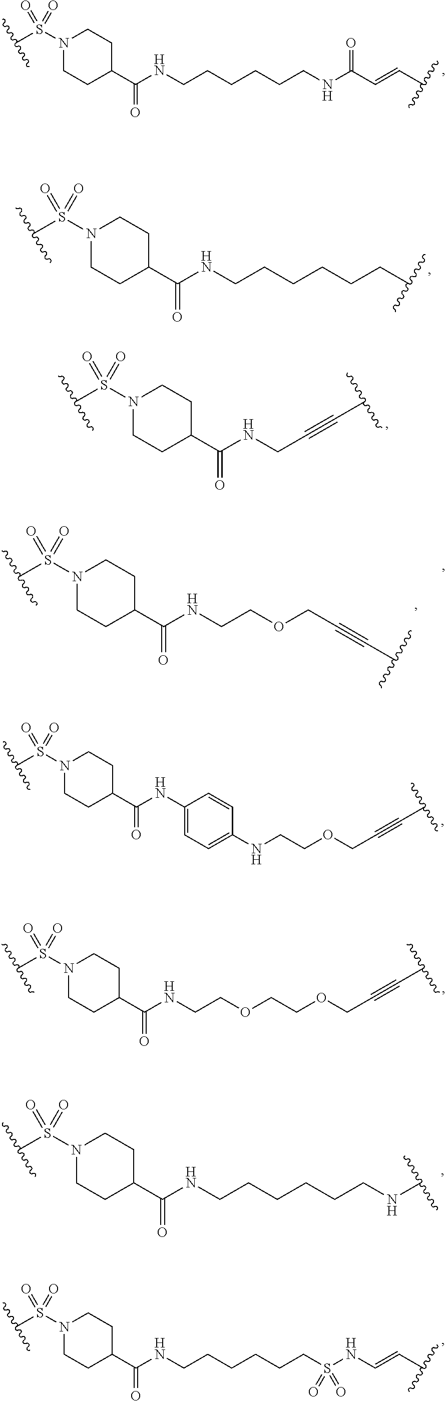

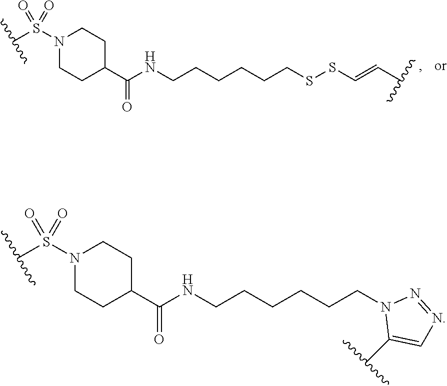 [SMILES: */C=C/SSCCCCCCNC(=O)C1CCN(S(*)(=O)=O)CC1 ; *c1cnnn1CCCCCCNC(=O)C1CCN(S(*)(=O)=O)CC1]

140. The method of claim 109, wherein L⁵⁰ is a substituted 2 to 10 membered heteroalkylene.

141. The method of claim 109, wherein L⁵⁰ is H

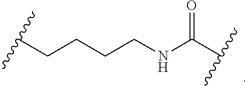 [SMILES: *CCCCNC(*)=O]

142. The method of claim 109, wherein R⁵⁰ is a fluorescent moiety.

143. The method of claim 142, wherein R⁵⁰ is a monovalent form of FAM, a monovalent form of VIC, a monovalent form of ABY, a monovalent form of JUN, a monovalent form of AF647, a monovalent form of Cy5, a monovalent form of AF676, or a monovalent form of Cy5.5.

144. The method of claim 109, wherein R² is hydrogen or —OH.

145. The method of claim 109, wherein R² is hydrogen.

146. The method of any one of claims 109 to 145, wherein R³⁰ is —OH.

147. The method of any one of claims 109 to 145, wherein R³⁰ is

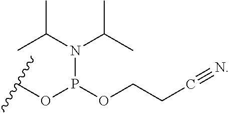 [SMILES: *OP(OCCC#N)N(C(C)C)C(C)C]

148. The method of any one of claims 109 to 145, wherein the 3′ blocking moiety is a monovalent form of dideoxycytidine (3′ddC), a monovalent form of dideoxyadenosine (ddA), 3′ Inverted dT, 3′ amino modifier, a monovalent form of QSY7, a monovalent form of QSY21, a monovalent form of QSY9, a monovalent form of BHQ1, a monovalent form of BHQ2, a monovalent form of BHQ3, a monovalent form of Dabcyl, a monovalent form of Dabsyl, a monovalent form of Eclipse, a monovalent form of BBQ-650, a monovalent form of Iowa Black RQ, a monovalent form of Iowa Black FQ,

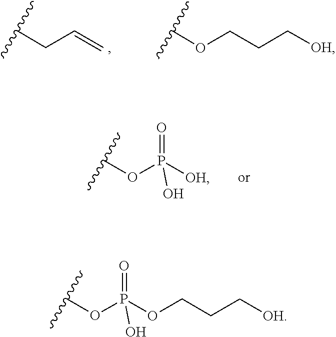

149. A composition for detecting nucleic acids in a sample, the composition comprising:
a first probe detectably labeled with a first label configured to generate a first emission signal,
a second probe detectably labeled with a second label configured to generate a second emission signal,
wherein the first and second probes have different sequences, and
wherein the first and second labels are identical and/or generate substantially identical emission
wherein under a first set of conditions, the first label generates a first emission signal that increases as a result of specific interaction of the first probe with a first nucleic acid target, and second label a second emission signal that is not increased as a result of specific interaction of the second probe with a second nucleic acid target; and
wherein under a second set of conditions different from the first set of conditions, the second emission signal is increases as a result of specific interaction of the second probe with the second nucleic acid target.

150. The composition according to claim 149, wherein the first probe is a cleavable probe.

151. The composition according to any one of claims 149 and 150, wherein the first probe includes a fluorophore and a quencher, and wherein the first probe is configured such that fluorescence from the fluorophore is quenched by the quencher until the probe is cleaved during an annealing/extension stage of the amplification process.

152. The composition according to any one of claims 149-151, wherein the first probe is a TaqMan probe.

153. The composition according to any one of claims 149-152, wherein the second probe is a non-cleavable probe.

154. The composition according to any one of claims 149-152, wherein the second probe comprises a stem-loop portion configured to form a stem-loop structure when the second probe is single-stranded.

155. The composition according to any one of claim 153 or 154, wherein the second probe comprises a fluorophore and a quencher spaced apart from one another such that the fluorophore is quenched when the second probe is single-stranded and unquenched when the second probe is incorporated into a double-stranded amplicon.

156. The composition according to claim 155, wherein the fluorophore is located at or near the 5′ end of the second probe and the quencher is 3′ of the fluorophore.

157. The composition of any one of claim 155 or claim 156, wherein both the fluorophore and the quencher are disposed at or near the stem loop portion of the second probe.

158. The composition of any one of claims 149-157, wherein the reaction mixture further comprises: a first primer pair complementary to a first nucleic acid target of the nucleic acids or its complement, the first nucleic acid target being configured to generate a first amplicon with which the first probe can hybridize; and a second primer pair complementary to a second nucleic acid target of the nucleic acids or its complement, the second nucleic acid target being configured to generate a second amplicon with which the second probe can hybridize.

159. The composition of claim 158, wherein the second primer pair includes a primer with a tail.

160. The composition of claim 159, wherein the tail forms the 5′ end of the primer with the tail.

161. The composition of any one of claim 159 or 160, wherein the second probe can hybridize to the tail or to its complement.

162. The composition of any one of claims 159-161, wherein the second primer pair further includes a non-tailed primer, and wherein a concentration of the primer with the tail in the reaction mixture is different from that of the non-tailed primer in the reaction mixture.

163. The composition of claim 162, wherein the concentration of the non-tailed primer is greater than that of the primer with the tail.

164. The composition of claim 163, wherein the concentration of the non-tailed primer is about 2× to about 30× greater than the concentration of the primer with the tail, or about 5× to about 25× greater than the concentration of the primer with the tail, or about 10× to about 20× greater than the concentration of the primer with the tail.

165. The composition of any one of claims 162-164, wherein the second probe is provided at a concentration that is different from the concentration of the primer with the tail and the concentration of the non-tailed primer.

166. The composition of claim 165, wherein the second probe is provided at a concentration that is greater than the concentration of the primer with the tail.

167. The composition of any one of claim 165 or 166, wherein the second probe is provided at a concentration that is less than the concentration of the non-tailed primer.

168. The composition of any one of claims 165-167, wherein the second probe is provided at a concentration that is about 2× to about 10× the concentration of the primer with the tail, or about 3× to about 7.5× the concentration of the primer with the tail.

169. A composition, comprising:
a primer pair complementary to a nucleic acid target or its complement for generating an amplicon, and
a non-cleavable probe configured to hybridize to the amplicon, the non-cleavable probe including a detectable label configured to provide an emission signal that corresponds to an amount of generated amplicon,
wherein the detectable label generates emission without cleavage of the non-cleavable probe during an amplification process including a series of thermal cycling stages that includes at least two different target temperatures.

170. The composition of claim 169, wherein the composition is a reaction mixture.

171. The composition of claim 169 or claim 170, wherein the non-cleavable probe comprises a stem-loop portion capable of forming a stem-loop structure when the non-cleavable probe is single-stranded.

172. The composition of any one of claims 159-170, wherein the non-cleavable probe comprises a fluorophore and a quencher spaced such that the fluorophore is quenched when the non-cleavable probe is single-stranded but enabled when the probe is incorporated into a double-stranded amplicon.

173. The composition of claim 172, wherein the fluorophore is located at or near the 5′ end of the probe and the quencher is 3′ of the fluorophore.

174. The composition of claim 172, wherein both the fluorophore and the quencher are at or near the stem loop portion of the probe.

175. The composition of any one of claims 169-174, wherein the primer pair includes a primer with a tail.

176. The composition of claim 175, wherein the tail forms the 5′ end of the primer with the tail.

177. The composition of claim 175 or claim 176, wherein the non-cleavable probe is configured to hybridize to the tail or to its complement.

178. The composition of claim 177, wherein a 3′ portion of the non-cleavable probe is configured to hybridize to the tail or its complement.

179. The composition of any one of claim 169, wherein the primer pair includes a primer with a tail and a non-tailed primer provided at different concentrations.

180. The composition of claim 179, wherein the non-tailed primer is provided at a greater concentration than the primer with the tail.

181. The composition of claim 180, wherein the non-tailed primer is provided at a concentration that is about 2× to about 30× the concentration of the primer with the tail, or about 5× to about 25× the concentration of the primer with the tail, or about 10× to about 20× the concentration of the primer with the tail.

182. The composition of claim 179, wherein a concentration of the non-cleavable probe in the reaction mixture is greater than a concentration of the primer with the tail in the reaction mixture.

183. The composition of claim 179, wherein the non-cleavable probe is provided at a concentration that is less than the concentration of the non-tailed primer.

184. The composition of any one of claim 183, wherein the non-cleavable probe is provided at a concentration that is about 2× to about 10× the concentration of the non-tailed primer, or about 3× to about 7.5× the concentration of the non-tailed primer.

185. The composition of any one of claim 169-184,
wherein the reaction mixture further comprises:
a primer pair complementary to a second nucleic acid target or its complement for generating a second amplicon, and
a cleavable probe configured to hybridize to the second amplicon, the cleavable probe including a detectable label configured to provide an emission signal that corresponds to an amount of generated second amplicon;
wherein subjecting the reaction mixture to the amplification process generates second amplicons, wherein the detectable label of the cleavable probe generates emission due to cleavage of the cleavable probe during the amplification process; and
wherein the method further comprises measuring the emission signal from the cleavable probe.

186. A kit comprising, the composition of any one of claims 149-185.
